(19) United States
(12) Patent Application Publication
(10) Pub. No.: US 2026/0034317 A1
(43) Pub. Date: Feb. 5, 2026

(54) INHALER AND CAPSULE FOR DELIVERING THYMIC STROMAL LYMPHOPOIETIN (TSLP)-BINDING ANTIBODIES

(71) Applicant: MedImmune Limited, Cambridge (GB)

(72) Inventor: Dan DEATON, Wilmington, DE (US)

(21) Appl. No.: 19/211,644

(22) Filed: May 19, 2025

Related U.S. Application Data
(60) Provisional application No. 63/649,781, filed on May 20, 2024.

(51) Int. Cl. A61M 15/00; A61K 9/00; A61K 47/18; A61P 11/06; C07K 16/24

(52) U.S. Cl. CPC A61M 15/0008; A61K 9/0073; A61K 47/183; A61M 15/002; A61M 15/003; A61P 11/06; C07K 16/244; C07K 2317/565

(57) ABSTRACT

A preloaded inhaler comprises a spin chamber in which a primary recess is configured to receive air to mix with contents of a capsule. The primary recess has a curved wall configured to allow rotation of the capsule. A secondary recess is configured to hold the capsule and is located within a bottom surface of the primary recess. A curved inlet channel allows air to travel therethrough. The curved inlet channel defines a curved recess and comprises a tangential section and a funnel section. A capsule containing a dry powder formulation which comprises an antigen binding fragment of an anti-thymic stromal lymphopoietin (TSLP) antibody is held in the spin chamber.

### SEQUENCE LISTING

This application contains a sequence listing which is submitted electronically and is hereby incorporated by reference in its entirety. The sequence listing submitted herewith is contained in the XML file created May 22, 2025 entitled “iTSLP-DPI-100-US01-NP-Sequence Listing.xml” and is 31,128 bytes in size.

### FIELD OF THE DISCLOSURE

The present disclosure relates generally to inhalers that are used to administer dry powder formulations of antigen binding fragments of thymic stromal lymphopoietin (TSLP) antibodies from a capsule, as well as methods of treating a TSLP-related condition such as asthma with the inhalers.

### BACKGROUND OF THE DISCLOSURE

Asthma affects an estimated 300 million people worldwide, including all age groups, and poses a serious burden on the health care system, and on society through loss of productivity at the workplace and disruption to the family. (“Pocket Guide for Asthma Management and Prevention,” Global Initiative for Asthma; 2019). Asthma causes symptoms such as wheezing, shortness of breath, chest tightness and cough that vary over time with their occurrence, frequency and intensity. Symptoms are often associated with bronchoconstriction, airway wall thickening and increased production of mucus. Asthma can have varying degrees of symptoms and be well controlled, or poorly controlled, based on number of attacks and severity.

Thymic stromal lymphopoietin (TSLP), an epithelial cell-derived cytokine produced in response to environmental and pro-inflammatory stimuli, leads to the activation of multiple inflammatory cells and downstream pathways. TSLP is increased in the airways of patients with asthma and correlates with Th2 cytokine and chemokine expression and disease severity. While TSLP is central to the regulation of Th2 immunity, it may also play a key role in other pathways of inflammation and therefore be relevant to multiple asthma phenotypes.

Delivery of antibodies to TSLP to a patient, in particular via inhalation, can provide an improved method of treatment for asthmatic patients, including those with mild asthma who may require daily, low-dose administration. Dry powder formulations comprising anti-TSLP antibody fragments suitable for inhalation for the treatment of asthma have been described in WO2021/083908. WO2017/042701 and WO2021/152488 describe methods for treating inflammatory or obstructive airway diseases, such as asthma, using an anti-TSLP antibody or an anti-TSLP antibody fragment. An anti-TSLP Fab with improved stability is described in WO2022/223514 for use in treating inflammatory diseases, such as asthma or COPD.

Inhalers are medical devices used to deliver a dose of medicament to a user by inhalation. There are numerous varieties of inhalers, but they all rely on the deliverance of the medicament into a user's lungs where the medicament may then be absorbed.

Dry powder inhalers are one such variety of inhaler. These deliver medicament to a user in the form of a dry powder, which is advantageous as this allows the medicament to reach further into the lungs than, for instance, metered dose or soft mist inhalers, thereby providing a greater therapeutic benefit to the user. Existing dry powder inhalers, such as those described in EP1270034A2 and US2007/295332A1, may comprise spin chambers within which the medicament contained within a capsule can be released and then mixed with air travelling through the spin chamber.

It is known that usability issues with inhalers may reduce treatment efficacy. For instance, studies investigating patient handling of inhalers for treating COPD suggests that device design and intended use of current inhalers is not intuitive to end users, which may lead to use errors and difficulties (see Molimard et al.: “Chronic obstructive pulmonary disease exacerbation and inhaler device handling: real-life assessment of 2935 patients”, European Respiratory Journal, 2017 49:1601794, DOI: 10.1183/13993003.01794-2016; and Chapman et al.: “Delivery characteristics and patients' handling of two single-dose dry-powder inhalers used in COPD”, International Journal of Chronic Obstructive Pulmonary Disease, 2011, 6:353-363, DOI: 10.2147/COPD.S18529).

Issues with the devices themselves may reduce treatment efficacy. For instance, spin chambers in existing dry powder inhalers may face issues such as powder migration from the spin chamber back into the inhaler, instead of out through the mouthpiece. This may affect the functions of other components of the inhaler. Existing spin chambers may also face issues with air flow due to their design. The surrounding geometry may cause disruption to the airflow, which may prevent the capsule from emptying its load and may result in a build-up of powder in areas that do not receive an adequate airflow. As a result, a user may not receive a full dose of medication. A disrupted airflow may also lead to fine particle agglomeration meaning that, even where a full dose of medication is delivered to the patient, it is not delivered to the preferred regions of the lungs.

The present disclosure has been devised in light of the above considerations.

### SUMMARY OF THE DISCLOSURE

According to a first aspect of the disclosure, there is disclosed a preloaded inhaler comprising a spin chamber having:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air to travel therethrough, the at least one curved inlet channel defining a curved recess and comprising a tangential section and a funnel section, wherein:
   at least a portion of the tangential section is substantially tangential to the curved wall of the primary recess;
   the tangential section is connected at a first end to an air inlet on an exterior surface of the spin chamber and at a second end to a first end of the funnel section, wherein the air inlet is configured to allow air to enter therethrough into the spin chamber; and
   the funnel section curves toward the primary recess and is connected at a second end to an entry point configured to allow air to enter therethrough into the primary recess, wherein the funnel section is downstream from the tangential section;
wherein the curved inlet channel is separated from the primary recess along a majority of its length by the curved wall of the primary recess;
wherein the preloaded inhaler comprises a capsule held in the spin chamber, the capsule containing a dry powder formulation which comprises an antigen binding fragment of an anti-thymic stromal lymphopoietin (TSLP) antibody.

According to a second aspect of the disclosure, there is disclosed a method of treating a disorder in a subject in need thereof comprising administering a dry powder formulation comprising an antigen binding fragment of an anti-TSLP antibody to the subject, wherein the formulation is administered from a capsule using a preloaded inhaler of the present disclosure which comprises the capsule. The disorder may be a TSLP-related condition.

According to a third aspect of the disclosure, there is disclosed a dry powder formulation comprising an antigen binding fragment of an anti-TSLP antibody; wherein the formulation is for use in therapy; and wherein the formulation is administered from a capsule using a preloaded inhaler of the present disclosure which comprises the capsule. The formulation may be for use in treating a TSLP-related condition.

According to a fourth aspect of the disclosure, a dry powder formulation comprising an antigen binding fragment of an anti-TSLP antibody is used in the manufacture of a medicament; wherein the formulation is administered from a capsule using a preloaded inhaler of the present disclosure which comprises the capsule. The medicament may be for the treatment of a TSLP-related condition.

According to a fifth aspect of the disclosure, there is disclosed a kit comprising:
(i) an unloaded inhaler comprising a spin chamber, the spin chamber comprising:
   a primary recess configured to receive air to mix with contents of a capsule, the primary recess having a curved wall configured to allow rotation of the capsule;
   a secondary recess configured to hold the capsule, the secondary recess located within a bottom surface of the primary recess; and
   at least one curved inlet channel configured to allow air to travel therethrough, the at least one curved inlet channel defining a curved recess and comprising a tangential section and a funnel section, wherein:
      at least a portion of the tangential section is substantially tangential to the curved wall of the primary recess;
      the tangential section is connected at a first end to an air inlet on an exterior surface of the spin chamber and at a second end to a first end of the funnel section, wherein the air inlet is configured to allow air to enter therethrough into the spin chamber; and
      the funnel section curves toward the primary recess and is connected at a second end to an entry point configured to allow air to enter therethrough into the primary recess, wherein the funnel section is downstream from the tangential section;
   wherein the curved inlet channel is separated from the primary recess along a majority of its length by the curved wall of the primary recess; and
(ii) one or more capsules for loading into the spin chamber of the inhaler, wherein the one or more capsules contains a dry powder formulation which comprises an antigen binding fragment of an anti-TSLP antibody.

According to a sixth aspect of the disclosure, there is disclosed a method of preparing a preloaded inhaler comprising loading a capsule into the spin chamber of an unloaded inhaler to form the preloaded inhaler, wherein the spin chamber comprises:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air to travel therethrough, the at least one curved inlet channel defining a curved recess and comprising a tangential section and a funnel section, wherein:
   at least a portion of the tangential section is substantially tangential to the curved wall of the primary recess;
   the tangential section is connected at a first end to an air inlet on an exterior surface of the spin chamber and at a second end to a first end of the funnel section, wherein the air inlet is configured to allow air to enter therethrough into the spin chamber; and
   the funnel section curves toward the primary recess and is connected at a second end to an entry point configured to allow air to enter therethrough into the primary recess, wherein the funnel section is downstream from the tangential section;
wherein the curved inlet channel is separated from the primary recess along a majority of its length by the curved wall of the primary recess; and
wherein the capsule contains a dry powder formulation which comprises an antigen binding fragment of an anti-TSLP antibody.

### DESCRIPTION OF THE DRAWINGS

FIG. 1A shows a perspective view of an inhaler in accordance with the present disclosure.

FIG. 1B shows a perspective view of an inhaler with an open drawer in accordance with the present disclosure.

FIG. 2 shows an exploded view of an inhaler in accordance with the present disclosure.

FIG. 3A shows a top view of an inhaler with an open drawer in accordance with the present disclosure.

FIG. 3B shows a cross-sectional side view of an inhaler in accordance with the present disclosure.

FIG. 4A shows a top view of a spin chamber of an inhaler in accordance with the present disclosure.

FIG. 4B shows a top view of a curved inlet channel of the spin chamber of FIG. 4A in accordance with the present disclosure.

FIG. 5A shows a top view of the spin chamber of FIG. 4A, with a first line indicating a cross-section of the spin chamber along a first curved inlet channel and a second line indicating a cross-section of the spin chamber along a second curved inlet channel in accordance with the present disclosure.

FIG. 5B shows a cross-sectional view of the spin chamber of FIG. 5A along the first line in accordance with the present disclosure.

FIG. 5C shows a cross-sectional view of the spin chamber of FIG. 5A along the second line in accordance with the present disclosure.

FIG. 6 shows an internal view of air flow through an inhaler in accordance with the present disclosure.

FIG. 7 shows a cross-sectional side view of the chimney and the drawer being held together in accordance with the present disclosure.

FIG. 8A shows the results of compressed bulk density as a function of leucine and trileucine in the dry powder formulations.

FIG. 8B shows the filling of capsules with dry powder formulations described herein.

FIG. 9 shows the results of specific surface area measured using BET, in m²/g, for microparticles of dry powder formulations in accordance with embodiments hereof.

FIG. 10A shows Pre-BD FEV1 (L)—mean change from baseline over time measured in the clinic, LS means—Results from Part B of Study described in Example 6.

FIG. 10B shows Pre-BD FEV1 (L)—mean change from baseline over time measured in the clinic, LS means—Results from the high dose arm (top—8 mg) or placebo (bottom) Part B of Study described in Example 6.

FIG. 11 shows ACQ-6 Change from Baseline by Dose Over Time, least squares (LS) means (80% confidence interval (CI))—Results from the high dose arm (top—8 mg) or placebo (bottom) Part B of Study described in Example 6.

FIG. 12 shows Scanning Electron Microscope (SEM) images of formulations tested in study 1.

FIGS. 13A and 13B show the Micro Flow Imaging (MFI) results for 10% bulk powder reconstituted in water for injection to the (FIG. 13A) 2.5 mg/ml protein and to (FIG. 13B) feedstock concentration (7.5 mg/ml protein). Results are expressed as particle counts per ml as an average of three replicates. Blue bars (on the left of each group)=TLTC PH 5 (21-WS-016); red bars (in the middle of each group)=TLTH pH 6 (21-WS-018); and green bars (on the right of each group)=TLTH pH 5 (21-WS-023).

FIGS. 14A and 14B show the MFI results for 40% bulk powder (BP) reconstituted in water for injection to the (FIG. 14A) 2.5 mg/ml protein and to (FIG. 14B) feedstock concentration (30 mg/ml protein). Results are expressed as particle counts per ml as an average of three replicates. Blue bars (on the left of each group)=TLTC PH 5 (21-WS-017); red bars (in the middle of each group)=TLTH pH 6 (21-WS-019); and green bars (on the right of each group)=TLTH pH 5 (21-WS-024).

FIGS. 15A, 15B, and 15C show the MFI results for (FIG. 15A) drug substance (DS) and (FIG. 15B) 10% and (FIG. 15C) 40% FAB1 reconstituted BP at feedstock protein concentration (7.5 mg/ml for 10% formulations and 30 mg/ml for 40% formulation). Figure legend A: blue bars (on the left of each group)=Drug substance citrate pH 5; red bars (in the middle of each group)=Drug substance histidine pH 6; green bars (on the right of each group)=Drug substance histidine pH 5. Figure legend B-C: Blue bars (on the left of each group)=TLTC pH 5 (21-WS-017); red bars (in the middle of each group)=TLTH pH 6 (21-WS-019); and green bars (on the right of each group)=TLTH pH 5 (21-WS-024).

FIGS. 16A, 16B, and 16C show the MFI results for 10% and 40% FAB1 BP at 2.5 mg/ml protein concentration across different formulations: (FIG. 16A) TLTC PH 5 at a concentration of 2.5 mg/ml, blue (on the left of each group)=10% (21-WS-016); red (on the right of each group)=40% (21-WS-017); (FIG. 16B) TLTH pH 6 at a concentration of 2.5 mg/ml, blue (on the left of each group)=10% (21-WS-018); red (on the right of each group)=40% (21-WS-019); (FIG. 16C) TLTH pH 5 at a concentration of 2.5 mg/ml, blue (on the left of each group)=10% (21-WS-023); red (on the right of each group)=40% (21-WS-024).

FIGS. 17A and 17B show (FIG. 17A) Next Generation Pharmaceutical Impactor (NGI) results for 10% FAB1 plotted by stage (blue on the left, red in the middle and green on right of each group) and (FIG. 17B) a summary of the results. The Next Generation Pharmaceutical Impactor (NGI) is as described in United States Pharmacopeia (USP)<601> Apparatus 6.

FIGS. 18A and 18B show (FIG. 18A) Next Generation Pharmaceutical Impactor (NGI) results for 10% FAB1 plotted by stage (blue on the left, red in the middle and green on right of each group) and (FIG. 18B) a summary of the results. The Next Generation Pharmaceutical Impactor (NGI) is as described in USP<601> Apparatus 6.

FIG. 19 shows study 2 40% FAB1 SEM images.

FIG. 20 shows study 2 10% FAB1 SEM images.

FIGS. 21A and 21B show the study 2 subvisible particles (SVP) detected by MFI for 10% FAB1 formulations reconstituted to (FIG. 21A) 2.5 mg/ml, or (FIG. 21B) the feedstock concentration of 7.5 mg/ml. Particle counts are expressed as particles/ml in BP for the listed sizes (no larger than (NLT) 2, 5, 10, 25 μm).

FIGS. 22A and 22B show the study 2 MFI results for 40% FAB1 formulations reconstituted to (FIG. 22A) 2.5 mg/ml, or (FIG. 22B) 30 mg/ml (feedstock concentration). Particle counts are expressed as particles/ml in BP for the listed sizes (no larger than (NLT) 2, 5, 10, 25 μm).

FIGS. 23A and 23B show (FIG. 23A) Next Generation Pharmaceutical Impactor (NGI) results for study 2 10% FAB1 plotted by stage; and (FIG. 23B) a summary of the results. The Next Generation Pharmaceutical Impactor (NGI) is as described in United States Pharmacopeia (USP)<601> Apparatus 6.

FIGS. 24A and 24B show (FIG. 24A) Next Generation Pharmaceutical Impactor (NGI) results for study 2 40% FAB1 plotted by stage, and (FIG. 24B) a summary of the results. The Next Generation Pharmaceutical Impactor (NGI) is as described in United States Pharmacopeia (USP)<601> Apparatus 6.

FIG. 25 shows study 3 SEM images.

FIGS. 26A and 26B shows study 3 MFI results 10% FAB1 reconstituted to (FIG. 26A) 2.5 mg/ml, or (FIG. 26B) the feedstock concentration of 7.5 mg/ml. Particle counts are expressed as particles/ml in BP for the listed sizes (no larger than (NLT) 2, 5, 10, 25 μm). Results are shown as the average of triplicate samples.

FIGS. 27A and 27B show study 3 SVP detected by MFI for 40% FAB1 formulations reconstituted to (FIG. 27A) 2.5 mg/ml, or (FIG. 27B) the feedstock concentration of 30 mg/ml. Particle counts are expressed as particles/ml in BP for the listed sizes (no larger than (NLT) 2, 5, 10, 25 μm). Results are shown as the average of triplicate samples.

FIGS. 28A and 28B show study 3 NGI results of (FIG. 28A) 10% FAB1 plotted by stage (Lot 21-WS-055, 056 and 057), (FIG. 28B) 40% FAB1 plotted by stage (Lot 21-WS-061, 058 and 059), and (FIG. 28C) a summary of the results. The Next Generation Pharmaceutical Impactor (NGI) is as described in United States Pharmacopeia (USP)<601> Apparatus 6.

FIG. 29 shows study 4 stability (1 month accelerated conditions) SEM images.

FIGS. 30A and 30B shows study 4 NGI results of 10% FAB1 (FIG. 30A) TLTH 1.3% His; (FIG. 30B) TLTH 3.15% His, (FIG. 30C) TLTH 5% plotted by stage. The Next Generation Pharmaceutical Impactor (NGI) is as described in United States Pharmacopeia (USP)<601> Apparatus 6.

FIGS. 31A to 31C shows study 4 NGI results of 40% FAB1 (FIG. 31A) TLTH 1.3% His; (FIG. 31B) TLTH 3.14% His, (FIG. 31C) TLTH 5% plotted by stage. The Next Generation Pharmaceutical Impactor (NGI) is as described in United States Pharmacopeia (USP)<601> Apparatus 6.

FIG. 32 shows perivascular/peribronchiolar mononuclear inflammatory cell infiltrates (arrows) with macrophage aggregates (triangles) in a mouse receiving 7.1 mg/kg/day FAB1 in TLTC, pH 6 with PS80 compared with a placebo control, labelled as “2 M 2001, placebo”.

FIG. 33 shows visible particles in the reconstituted FAB1 TLTC, pH 6 with PS80 in water.

FIG. 34 shows representative images FAB1-related lung pathology for the second toxicity study, at the indicated dose levels (placebo, 4 mg/kg, and 9.2 mg/kg). Arrows for the lung images point to mononuclear cell (MNC) infiltrates.

FIG. 35 lists the pathology results for the second toxicity study.

FIG. 36 FeNO Mean Change from Baseline by Dose Over Time, GLS Means (80% confidence interval (CI))—Results from Part B of Study described in Example 6.

FIG. 37 FAB1 popPK model schematic. ka=absorption rate constant, F1 and F2=first order and zero order absorption bioavailability respectively, D2=duration of zero order absorption, CL=Clearance, Q1 and Q2=intercompartmental clearances, Vc, V1 and V2=Volume of the central compartment, peripheral compartment 1 and peripheral compartment 2 respectively. Observations are the dotted line.

FIG. 38 FAB1 predicted serum (dashed line) and lung concentration (solid line) following inhaled 0.4, 2 and 8 mg QD administration. Grey shaded area is a visualisation aid to separate serum and lung predictions. Horizontal dotted line is the predicted lung Cave concentration of tezepelumab following SC 210 mg administration (Q4W).

FIG. 39 shows a Phase 2b protocol design for testing the efficacy of an anti-TSLP Fab fragment.

FIG. 40 shows the results of a Next Generation Impactor (NGI) study on an inhaler of the present disclosure compared with a monodose inhaler. Both inhalers comprised a capsule containing 20 mg of a dry powder formulation comprising 0.2 mg of FAB1.

FIG. 41 shows the results of a Next Generation Impactor (NGI) study on an inhaler of the present disclosure compared with a monodose inhaler. Both inhalers comprised a capsule containing 20 mg of a dry powder formulation comprising 4 mg of FAB1.

### DETAILED DESCRIPTION OF THE DISCLOSURE

### Dry Powder Inhalers

The inhalers disclosed herein comprise a spin chamber in which a dry powder-containing capsule may be present. The capsule contains a dry-powder formulation which comprises an antigen binding fragment of a TSLP antibody. Both the capsule and dry-powder formulation are described in greater detail below.

In some instances of the disclosure, the inhaler is a preloaded inhaler. When the inhaler is in its “preloaded” form, the capsule is held in the spin chamber and forms part of the preloaded inhaler device. The capsule will generally be held within the secondary recess of the spin chamber, in particular when the preloaded inhaler is not in use. During use, the capsule may leave the secondary recess and enter the primary recess where it may spin due to airflow through the preloaded inhaler.

The inhaler may also exist as an unloaded inhaler. When the inhaler is in an “unloaded” form, the capsule is not present in the spin chamber and does not form part of the “unloaded inhaler” device. Nevertheless, an unloaded inhaler is ready for the capsule to be loaded into the chamber. According to the present disclosure, the unloaded inhaler forms part of a kit, where the kit further comprises one or more capsules containing the dry powder formulation.

It will be understood that references to an “inhaler” or to particular features of an “inhaler”, without specifying whether the inhaler is preloaded or unloaded, relate to both the preloaded and unloaded forms.

The spin chambers that are used in inhalers of the present disclosure comprise a primary recess configured to receive air to mix with contents of a capsule. The primary recess has a curved wall configured to allow rotation of the capsule.

The spin chamber also comprises a secondary recess configured to hold the capsule. The secondary recess is located within a bottom surface of the primary recess.

At least one curved inlet channel is present in the spin chamber and is configured to allow air to travel therethrough. The at least one curved inlet channel defines a curved recess and comprises a tangential section and a funnel section. At least a portion of the tangential section is substantially tangential to the curved wall of the primary recess. The tangential section is connected at a first end to an air inlet on an exterior surface of the spin chamber and at a second end to a first end of the funnel section, with the air inlet configured to allow air to enter therethrough into the spin chamber. The funnel section curves toward the primary recess and is connected at a second end to an entry point configured to allow air to enter therethrough into the primary recess. The funnel section is downstream from the tangential section. The curved inlet channel is separated from the primary recess along a majority of its length by the curved wall of the primary recess.

The spin chamber may have a longitudinal axis extending from a top of the spin chamber, down through the primary and secondary recesses, to a bottom of the spin chamber. The spin chamber may comprise a top surface located at the top of the spin chamber with respect to the longitudinal axis. The primary recess may be proximate to the top of the spin chamber along the longitudinal axis, and the secondary recess may be proximate to the bottom of the spin chamber along the longitudinal axis. The bottom surface of the primary recess may face the top of the inhaler with respect to the longitudinal axis. The spin chamber may be configured so that in use air flows in from the air inlet, through the at least one curved inlet channel, through the primary recess and out through an outlet of the inhaler.

The tangential section may comprise a first portion and a second portion. The first portion may extend from the first end of the tangential section to a point between the first end and the second end of the tangential section. The second portion may extend from the point between the first end and the second end of the tangential section to the second end of the tangential section. The second portion may be downstream from the first portion. The first portion may be widest near the air inlet. The second portion may be of a substantially uniform width.

The at least one curved inlet channel may comprise an inner wall and an outer wall. The inner wall may substantially follow an outline of the primary recess. The inner wall may extend along an entirety of the tangential section and along at least a portion of the funnel section. The outer wall may be substantially straight in the first portion of the tangential section of the at least one curved inlet channel.

The primary recess may be substantially cylindrical. This may help to encourage rotation of a capsule during inhalation.

The secondary recess may be substantially obround-shaped with a length that is greater than its width, such that the secondary recess is configured to receive a capsule horizontally relative to the longitudinal axis. This may help to ensure that a capsule can be perforated at both ends, thus resulting in a quicker and more efficient release of medication from the capsule.

The at least one curved inlet channel may have a length that is greater than a radius of the primary recess.

The top surface of the spin chamber may be curved in a convex manner such that a depth along the longitudinal axis of the at least one curved inlet channel varies along its length. This curvature enables a drawer containing the spin chamber to be closed into the inhaler via a hinge mechanism, which results in a simplified user experience.

The at least one curved inlet channel may comprise two curved inlet channels. This results in a greater air flow, which helps to lift the capsule and allow its contents to mix with the air.

The two curved inlet channels may be disposed on opposing sides of the primary recess. This means that air may interact with the capsule from both sides, which helps to lift the capsule and allow its contents to mix with the air.

The tangential sections of each opposing curved inlet channel may be opposite each other across the primary recess and the funnel sections of each opposing curved inlet channel may be opposite each other across the primary recess.

A first curved inlet channel of the two curved inlet channels may have a greater depth along the longitudinal axis in its tangential section than in its funnel section and a second curved inlet channel of the two curved inlet channels has a greater depth in its funnel section than in its tangential section.

A cross-sectional area of the air inlet of a first of the two curved inlet channels may be substantially equal to a cross-sectional area of the air inlet of a second of the two curved inlet channels. This may help to ensure a balanced air flow through both curved inlet channels, thus encouraging a stable cyclone to be generated in the spin chamber.

The at least one curved inlet channel may be configured such that in use, air feeds into the primary recess, thereby causing the capsule to be lifted out of the secondary recess and to spin in the primary recess. This helps to release the contents of the capsule quickly and efficiently, resulting in a greater likelihood of successful delivery.

A bottom surface of the at least one curved inlet channel may be substantially level with the bottom surface of the primary recess with respect to the longitudinal axis.

The primary recess may extend downwards from the top surface of the spin chamber along the longitudinal axis, and the at least one curved inlet channel may define a curved recess extending downwards from the top surface of the spin chamber.

The inhalers disclosed herein may be configured to allow air to flow in from the air inlet, through the at least one curved inlet channel, through the primary recess and out through an outlet of the inhaler.

Additionally, the outlet of the inhaler may comprise a mouthpiece. Thus, the inhaler is typically used to deliver the dry powder formulation orally.

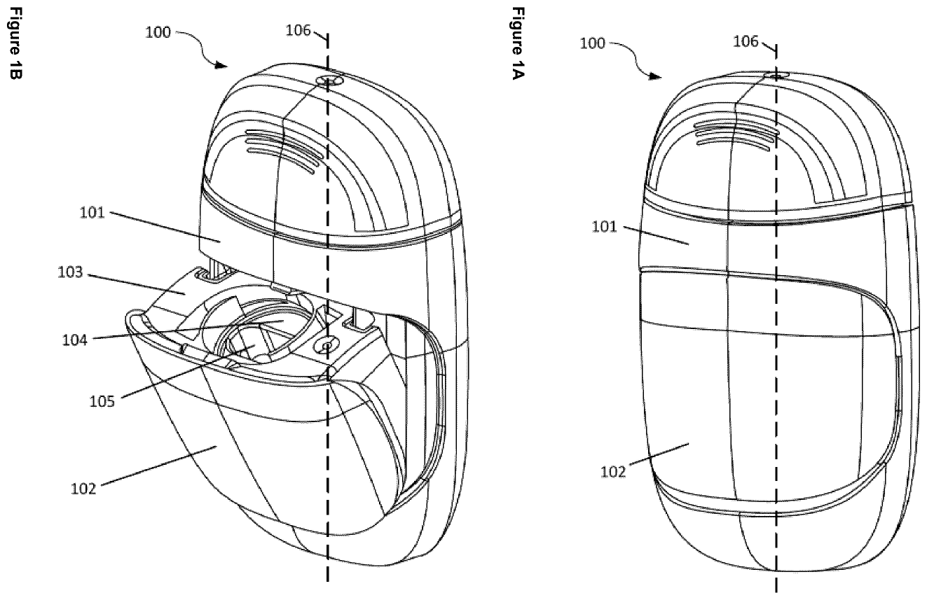

Embodiments of the inhaler will now be described, by way of example only, with reference to FIGS. 1 to 7.

FIGS. 1A-B show perspective views of an inhaler 100. The inhaler 100 comprises a main body 101 and a drawer 102. The drawer 102 may be coupled to the main body by way of a hinge mechanism, which allows the drawer 102 to open out of and close into the main body 101 of the inhaler 100. This enables the drawer 102 to be accessed without having to remove it from the main body 101 entirely. The inhaler 100 may comprise a longitudinal axis 106, with the top of the inhaler 100 being positioned above the bottom of the inhaler 100 with respect to the longitudinal axis 106.

In FIG. 1A, the drawer 102 is shown as being in a closed position. In the closed position, a longitudinal axis of the drawer 102 may directly correspond to the longitudinal axis 106 of the inhaler 100. In FIG. 1B, the drawer 102 is shown as being in an open position, such that the components of the drawer 102 are visible. In the open position, the drawer 102 is angled outwards such that the longitudinal axis of the drawer is angled away from the longitudinal axis 106 of the inhaler 100.

The main body 101 is configured to act as a framework for the inhaler 100 and enclose the majority of the other components of the inhaler 100. The main body 101 may comprise polybutylene terephthalate (PBT) and at least a portion of the main body 101 may comprise wax-lubricated PBT. The main body 101 may comprise at least one air inlet to allow air to flow through the inhaler 100. The drawer 102 is configured to be opened out of and closed into the main body 101. More specifically, the spin chamber 103 of the drawer 102 is configured to receive a capsule and to allow the contents of the capsule to mix with air during inhalation. The contents of the capsule is a dry powder formulation which comprises an antigen binding fragment of a TSLP antibody and is described in greater detail below.

The drawer 102 comprises a spin chamber 103, which is located near the top of the drawer 102 with respect to the longitudinal axis 106. The spin chamber may comprise a primary recess 104 and a secondary recess 105. The primary recess 104 may extend downwards from the top surface of the spin chamber 103. The secondary recess 105 may be located within a bottom surface of the primary recess 104. As such, the secondary recess 105 can be considered as an extension of the primary recess 104. The primary recess 104 may be substantially cylindrical in shape, which may help to encourage rotation of a capsule during inhalation. The secondary recess 105 may be substantially obround in shape, with a length that is greater than its width. The primary recess 104 has a larger volume than the secondary recess 105.

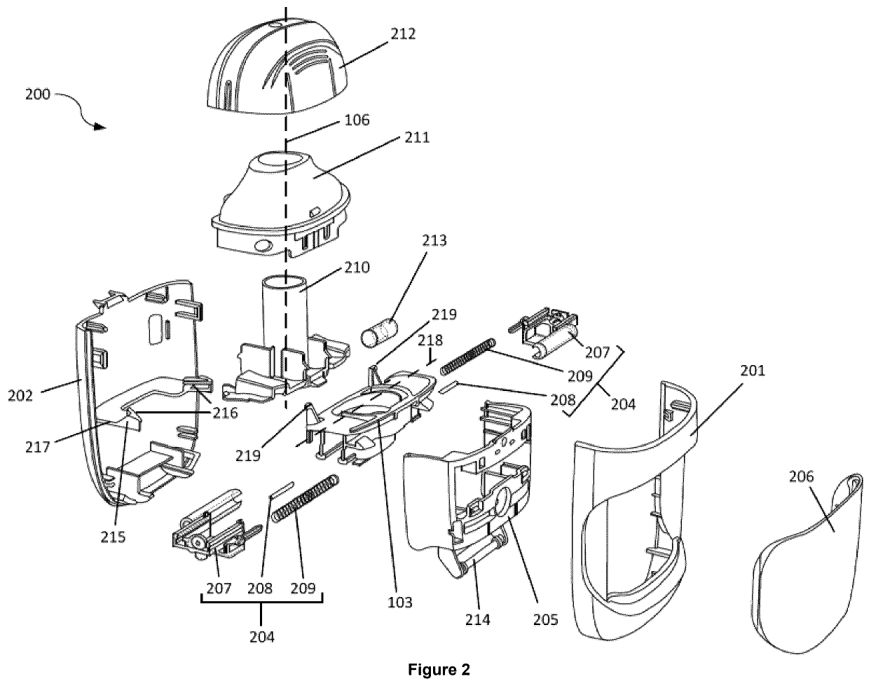

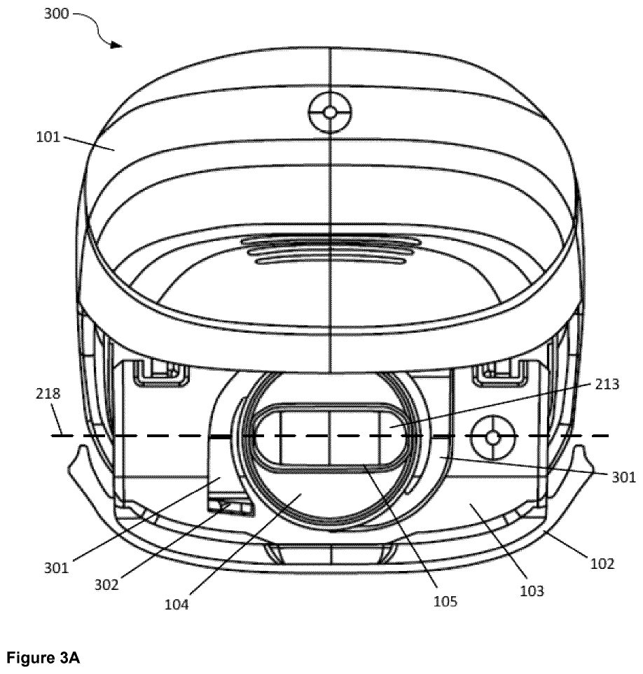

The secondary recess 105 is configured to receive the capsule. The obround shape of the secondary recess 105 enables the capsule to be received horizontally. This may help to ensure that a capsule can be perforated at both ends, thus resulting in a quicker and more efficient release of medication from the capsule. The process of perforation will be described with reference to FIG. 2. The primary recess 104 is configured to allow the contents of the capsule to mix with air during inhalation.

Use of the inhaler 100 begins with the insertion of a capsule into the drawer 102 of the inhaler in its unloaded form. The capsule is placed into the secondary recess 105 and the drawer 102 is closed into the main body 101. Closing the drawer 102 causes the capsule to be perforated, which will be described in greater detail with respect to FIG. 2. Thus, the preloaded inhaler of the present disclosure may comprise a perforated capsule. As the user inhales through the mouthpiece (not shown), the air flow through the spin chamber 103 causes the capsule to be lifted out of the secondary recess 105 into the primary recess 104, where it may spin such that its contents may mix with air flowing through the spin chamber 103. This mixture is then inhaled by the user. The drawer 102 may then be opened and the capsule removed thereby providing an unloaded inhaler once again.

The inhaler 100 and its use will be described in greater detail with respect to FIG. 2.

FIG. 2 shows an exploded view of an inhaler 200. The inhaler 200 may correspond to the inhaler 100 from FIGS. 1A-B and may therefore comprise a main body and a drawer, corresponding respectively to the main body 101 and the drawer 102 from FIGS. 1A-B.

More specifically, the main body of inhaler 200 may comprise a front casing 201 and a rear casing 202. The front casing 201 and rear casing 202 are connected to each other to provide a space within which other components of the inhaler 200 may be located. Each of the front casing 201 and rear casing 202 comprises an inner surface and an outer surface. When the front casing 201 and rear casing 202 are connected to each other, the two inner surfaces face inwards towards each other, while both outer surfaces face outwards. The front casing 201 and rear casing 202 both extend upwards along a longitudinal axis that may correspond to the longitudinal axis 106 from FIGS. 1A-B.

The front casing 201 comprises an aperture through which the drawer may move between an open position and a closed position. When the drawer is in the closed position, an outer surface of the drawer casing substantially fills the aperture of the front casing 201. When the drawer is in the open position, the components of the drawer are exposed, such that a capsule 213 may be inserted into or removed from the drawer. As described, the capsule 213 comprises medicament in the form of a dry powder formulation.

The rear casing 202 may comprise at least one wedge 215, the at least one wedge 215 comprising an inner side 216 and being connected to a flexible arm 217. FIG. 2 shows an embodiment in which the rear casing 202 comprises two wedges 215, each comprising an inner side 216 and each attached to a separate flexible arm 217, but it is to be understood that fewer or more wedges 215 and flexible arms 217 are possible. In FIG. 2, the flexible arms 217 protrude outwards from the inner surface of the rear casing 202 along an axis that is substantially perpendicular to the longitudinal axis 106 of the rear casing 202.

The drawer of inhaler 200 may comprise a spin chamber 103, perforating means 204, a supporting framework 205 and a drawer casing 206. The spin chamber 103 may correspond to the spin chamber 103 from FIG. 1B and may comprise a transverse axis 218 that is substantially perpendicular to the longitudinal axis 106 of the inhaler and also substantially perpendicular to the axis along which the flexible arm 217 protrudes. The spin chamber 103, in addition to comprising a primary recess 104 and a secondary recess 105 for receiving a capsule 213, may also comprise at least one guide post 219. FIG. 2 shows an embodiment in which the spin chamber 103 comprises two guide posts 219, each located on opposing sides of the spin chamber 103 along the transverse axis 218. The guide posts 219 may extend upwards from a top surface of the spin chamber 103 substantially along the longitudinal axis 106.

The spin chamber 103 is coupled to the perforating means 204, which are positioned at a side of the spin chamber 103 along the transverse axis 218. The perforating means 204 are positioned so as to be able to move along the transverse axis 218 between a resting position and a perforating position. The perforating position is a position within the secondary recess 105 where the perforating means 204 may perforate the capsule 213. When in the resting position, the perforating means are further away from the centre of the spin chamber 103 than when in the perforating position. The spin chamber 103 may comprise rails to allow the perforating means 204 to slide along the transverse axis 218 between the resting position and the perforating position. The perforating means 204 may comprise grooves that interact with the rails of the spin chamber 103 to enable this movement. The spin chamber 103 may also comprise a T-rail (not shown) that helps to maintain alignment of the spin chamber 103 and the perforating means 103. The spin chamber 103 may further comprise perforating means retention clips (not shown) that prevent the perforating means 204 from moving outwards beyond their resting position along the transverse axis 218.

The spin chamber 103 and perforating means 204 may be coupled to the supporting framework 205, which holds the spin chamber 103 in a set position within the drawer. The supporting framework 205 also encloses the perforating means 204 within the drawer and may also help to prevent the perforating means 204 from moving outwards beyond their resting position along the transverse axis 218. A front side of the supporting framework 205 is attached to the drawer casing 206. The supporting framework may also comprise a hinge 214, which may be connected to the front casing 201 by way of a hook mechanism. The hook mechanism may have a substantially semi-circular cross section. The hinge 214 may also be connected to the rear casing 202. The presence of the hinge 214 may enable the drawer to be opened out of and closed into the main body while remaining attached to the main body. This enables the drawer to be accessed without having to remove it from the main body entirely.

The perforating means 204 may comprise a cam post 207, a needle 208 and a spring 209. The cam post 207 is coupled to a non-perforating end of the needle 208 and to a first end of the spring 209. The needle 208 and spring 209 both extend away from the cam post 207 along the transverse axis 218. The needle 208 may be encompassed by the spring 209, or it may be positioned away from the spring 209.

The second end of the spring 209 may be coupled to an inner portion of the perforating means 204, whereas the perforating end of the needle 208 is not directly connected to any other part of the inhaler. The spring 209 is in a rest state when the drawer is in the open position and when the drawer is in the closed position, but may be compressed as the drawer moves from the open position to the closed position, as will be described in greater detail.

FIG. 2 shows the perforating means 204 as comprising two sets of cam posts 207, needles 208 and springs 209, with each set located along the transverse axis 218 on opposing sides of the spin chamber 103, although the preceding paragraphs have so far described only one cam post 207, one needle 208 and one spring 209. It is to be understood that the inhaler may function with one cam post 207, one needle 208 and one spring 209, or with two cam posts 207, two needles 208 and two springs 209. The only requirements are that the perforating means comprises at least one cam post 207, at least one needle 208 and at least one spring 209. In an embodiment, the perforating means comprises two cam posts 207, two needles 208 and two springs 209, as shown in FIG. 2. In this embodiment, each of the two springs 209 may be coupled to the same inner portion of the perforating means at their respective second ends.

The two needles 208 may comprise a pair of opposing needles 208, each needle 208 coupled to a respective spring 209. The use of two opposing needles 208 may result in two perforations of the capsule 213. This decreases the time required for the contents of the capsule 213 to be removed from the capsule 213 through inhalation, since there will be two holes created in the capsule 213. The opposing needles 208 may be configured to perforate the capsule 213 at the same time. This helps to ensure an efficient and timely emptying of the capsule 213, since both holes will be created at the same time.

As described, the secondary recess 105 may be substantially obround-shaped. The needles 209 may be configured to enter opposing ends of the secondary recess 105 and subsequently perforate opposing ends of the capsule 213. This helps to ensure an efficient and timely emptying of the capsule, since this minimises the distance the contents of the capsule 213 will have to travel in order to exit the capsule 213.

As described, the perforating means 204 are configured to move along the transverse axis 218 between a resting position and a perforating position. More specifically, the cam post may be configured to transversely slide against the bias of the spring 209, which causes the spring 209 to compress. Since the needle 208 is attached to the cam post 207, the needle 208 may also be configured to transversely slide against the bias of the spring 209.

The movement of the drawer from an open position to a closed position may cause the perforating means 204 to move from the resting position to the perforating position.

The inhaler 200 may further comprise an inhalation chimney 210. The inhalation chimney 210 may comprise a hollow tube through which air and the dry powder formulation may pass. The inhalation chimney 210 is positioned along the longitudinal axis 106 near the top of the inhaler, such that when the drawer is in the closed position, the inhalation chimney is directly above the spin chamber 103. The hollow tube extends along the longitudinal axis 106. The bottom of the hollow tube of the chimney 210 aligns with the primary recess 104 and secondary recess 105 of the spin chamber 103. When the drawer is in the closed position, the inhalation chimney 210 and the spin chamber 103 together define a space within which the contents of the capsule 213 may be spun as air travels through the inhaler 200.

The inhalation chimney 210 may also comprise at least one protruding rib along which the at least one guide post 219 of the spin chamber 103 may pass. The at least one protruding rib may extend outwards along the transverse axis 218. For example, there may be two protruding ribs on opposing sides of the inhalation chimney 210. The number of protruding ribs is the same as the number of guide posts 219.

The inhalation chimney 210 may also comprise at least one drawer retention clip (not shown). The at least one drawer retention clip may be situated near the bottom of the inhalation chimney 210 on the side that is closest to the rear casing 202. In an embodiment, the at least one drawer retention clip comprises two drawer retention clips on opposing sides of the inhalation chimney 210 with respect to the transverse axis 218.

The inhaler 200 may also comprise a mouthpiece 211. The mouthpiece 211 is positioned on top of the inhalation chimney 210 and comprises an aperture through which the inhalation chimney 210 may extend. The inhalation chimney 210 may move upwards along the longitudinal axis 106 such that a top surface of the inhalation chimney 210 is higher than a top surface of the mouthpiece 211 with respect to the longitudinal axis 106. The inhalation chimney 210 may move downwards along the longitudinal axis 106 such that the top surface of the inhalation chimney 210 is at the same level as the top surface of the mouthpiece 211 with respect to the longitudinal axis 106.

The mouthpiece 211 is attached to the front casing 201 and rear casing 202 of the inhaler 200.

The inhaler 200 may also comprise a cap 212. The cap 212 is positioned on top of the mouthpiece 211 and may cover the entire top surface of the mouthpiece 211. The cap 212 is attached to the mouthpiece 211 by way of a hinge mechanism that enables the cap 212 to either allow access to the mouthpiece 211 or to cover and prevent access to the mouthpiece 211.

With reference now to the function of the components of the inhaler 200, the front casing 201 and rear casing 202 are configured to act as the main body of the inhaler 200. The front casing 201 and rear casing 202 are joined to define an outer housing of the inhaler 200, within which other components may be enclosed.

The spin chamber 103, as has been described with reference to FIG. 1B, is configured to receive a capsule 213 and to allow air to mix with the contents of the capsule 213. More specifically, the secondary recess 105 of the spin chamber 103 is configured to receive the capsule 213. As air flows through the inhaler 200, the capsule 213 may be lifted out of the secondary recess 105 and into the primary recess 104, where the capsule 213 may spin around in order to allow its contents to mix with the air.

The perforating means 204 are configured to perforate the capsule 213, thus releasing the contents of the capsule 213 and allowing them to mix with air so that they may be inhaled by a user. More specifically, the perforating means 204 are configured to move inwards along the transverse axis 218 from a resting position to a perforating position as the drawer moves from an open position to a closed position. When at the perforating position, which occurs shortly before the drawer is in the closed position, the perforating means 204 are configured to perforate the capsule 213 and then move back from the perforating position to the resting position. When the drawer is in the closed position, the perforating means 204 are in the resting position. As the drawer moves from the closed position to the open position, the perforating means are configured to remain in the resting position.

The perforating means 204 are configured to interact with a portion of the main body of the inhaler 200 as the drawer moves between the open position and the closed position, which causes the perforating means to move away from their resting position towards their perforating position. More specifically, the perforating means are configured to interact with the wedge 215, which is attached to the flexible arm 217.

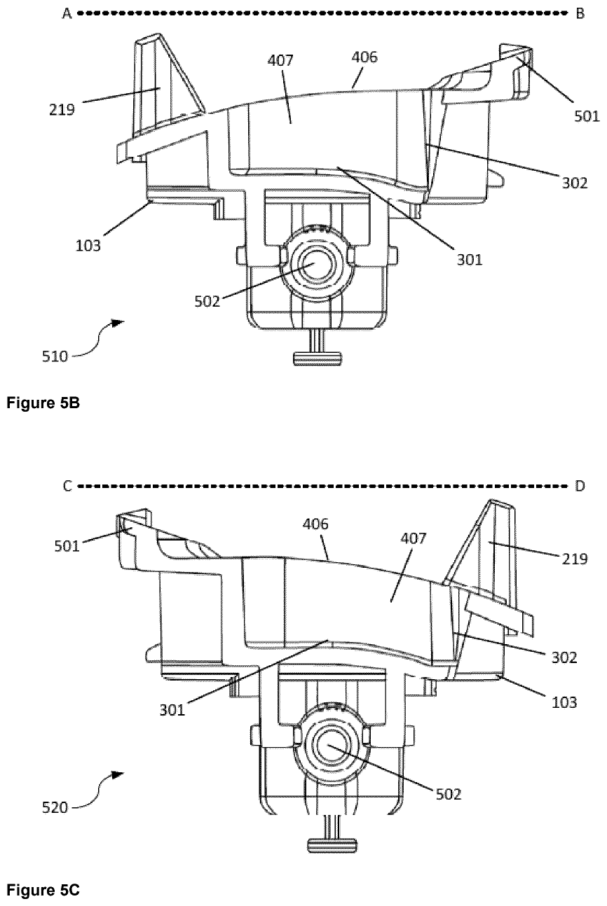

As the drawer moves into the main body of the inhaler 200 from the open position to the closed position, the cam post 207 of the perforating means 204 is configured to slide along the inner side 216 of the wedge 215. The angle of this inner side 216 causes the cam post 207 to be pushed inwards towards the centre of the spin chamber 103 along the transverse axis 218, against the biasing of the spring 209. This compresses the spring 209, which subsequently provides a resistive force. This helps to keep the other components of the perforating means 204 in the desired position. The needle 208, which is attached to the cam post 207, also moves inwards towards the centre of the spin chamber 103 and passes through a small aperture in the side of the spin chamber 103. Further details of this small aperture will be discussed with reference to FIGS. 5B-C. By the time the cam post 207 has reached the end of the inner side 216, the perforating means 204 have moved along the transverse axis 218 and have reached their perforating position. When in the perforating position, the needle 208 has extended through the small aperture in the side of the spin chamber 103 and into the secondary recess 105, where it may perforate the capsule 213.

This means that a capsule 213 can be perforated as the drawer is closed into the main body, rather than this being a separate step that must be initiated after the drawer has been closed. This makes use of the inhaler 200 easier and quicker for a user and also minimises the risk of a user failing to perforate a capsule (e.g. by not pressing a button hard enough), since the perforating means 204 must reach the perforating position in order for the drawer to successfully close.

Once the perforating position has been reached and the capsule 213 has been perforated, the perforating means 204 are configured to pass over the edge of the inner side 216 of the wedge 215 and in doing so return to the resting position. The compressed spring 209 decompresses and returns to its rest position. In doing so, the spring 209 pushes the needle 208 out of the secondary recess 105 such that the perforating means 204 can return to the resting position so that they are in the correct position for a subsequent opening of the drawer. At this point, the drawer is in the closed position. Beneficially, this means that a user does not have to manually reset the perforating means 204.

As the drawer is moved from a closed position to an open position, the perforating means 204 are configured to interact with the wedge 215, but in a different manner to the interaction that takes place when the drawer is being closed. As the drawer moves away from the closed position, the perforating means 204 are configured to travel over a top surface of the wedge 215. More specifically, the cam post 207 travels over the top surface of the wedge 215, which causes the flexible arm 217 to move downwards along the longitudinal axis 106 towards the bottom of the inhaler. As the cam post 207 travels over the wedge 215, the perforating means 204 remain in the resting position with respect to the transverse axis 218, meaning that the spring remains in the rest state. Once the cam post 207 has travelled over the top surface of the wedge 215, the wedge 215 moves back up to its normal resting position so that it is in the correct position for a subsequent closing of the drawer.

The inhalation chimney 210 is configured to move downwards with respect to the longitudinal axis 106 as the drawer moves from an open position to a closed position and is configured to move upwards with respect to the longitudinal axis 106 as the drawer moves from a closed position to an open position. More specifically, the guide posts 219 of the spin chamber are configured to interact with the inhalation chimney 210 as the drawer moves between the open and closed positions, which causes the inhalation chimney 210 to move upwards or downwards. When the drawer is in the closed position, the drawer retention clips are configured to hold the guide posts 219 in position, such that a force is required to move the guide posts 219 out of this position and open the drawer.

The mouthpiece 211 is configured to be inserted into a user's mouth during inhalation. The cap 212 is configured to cover the mouthpiece 211 when the inhaler 200 is not in use, thus preventing any foreign substances from entering the inhaler 200 through the mouthpiece 211.

In order to use the inhaler 200, a user inserts a capsule 213 into the secondary recess 105 of the spin chamber 103 of the inhaler in its unloaded form. The drawer must be in the open position for this to take place, since the spin chamber 103 cannot be accessed if the drawer is in the closed position. Once the capsule 213 is positioned within the secondary recess 105, the user may push the drawer inwards to move it from the open position towards the closed position thereby forming a preloaded inhaler. As the drawer moves towards the closed position, the perforating means 204 interact with the wedge 215, which causes them to slide along the inner side 216 of the wedge 215 and to move inwards along the transverse axis 218, as has been described.

The movement of the drawer causes the cam post 207 and the needle 208 to move inwards towards the centre of the spin chamber 201 along the transverse axis 218. As the cam post 207 approaches the edge of the wedge 215, the needle 208 perforates the capsule 213. The perforating means 204 then pass over the edge of the wedge 215 and return to the resting position.

The spin chamber 103 also interacts with the inhalation chimney 210 as the drawer moves from the open position towards the closed position. More specifically, the guide posts 219 of the spin chamber 103 slide along the protruding ribs of the inhalation chimney before travelling over sealing ramps of the inhalation chimney 210 as the drawer approaches the closed position. As the guide posts 219 travel over the sealing ramps, they cause the inhalation chimney 210 to be pulled downwards along the longitudinal axis 106, such that a bottom surface of the inhalation chimney 210 is brought closer to a top surface of the spin chamber 103. The two surfaces may be brought into contact, or a small gap may remain between them when the drawer is in the closed position. When the drawer is in the closed position, the inhalation chimney 210 has been pulled down such that a top surface of the inhalation chimney 210 is level with a top surface of the mouthpiece 211 with respect to the longitudinal axis 106. Drawer retention clips hold the guide posts 219 in position, such that the inhalation chimney 210 is held in position with respect to the spin chamber 103.

As discussed above, when the drawer is in the closed position, the perforating means 204 have perforated the capsule 213 and returned to the resting position and the inhalation chimney 210 has moved down towards the spin chamber 103. At this stage, the user may open the cap 212 to expose the mouthpiece 211. By placing the inhaler 200 in their mouth, tilting it and inhaling, an air flow may be generated through the inhaler 200. The air flow may lift the perforated capsule 213 out of the secondary recess 105 and into the primary recess 104 of the spin chamber 103, where it may cause the capsule 213 to spin and the contents of the capsule 213 to mix with the air. The resulting mixture of the contents of the capsule 213 and the air may then pass through the hollow tube of the inhalation chimney 210, through the aperture of the mouthpiece 211 and into the mouth of the user.

Upon successful inhalation, the drawer of the inhaler 200 may then be opened so that the capsule 213 may be removed thereby providing an unloaded inhaler once again. As the drawer is pulled outwards, the guide posts 219 push the drawer retention clips away and travel back over the sealing ramps. The guide posts 219 then interact with the protruding ribs of the inhalation chimney 210, which pushes the inhalation chimney 210 upwards with respect to the longitudinal axis 106.

At the same time, the perforating means 204 travel over the wedges 215. This movement pushes the wedges 215 downwards with respect to the longitudinal axis 106. The perforating means 204 therefore remain in the resting position as they travel over the wedges 215.

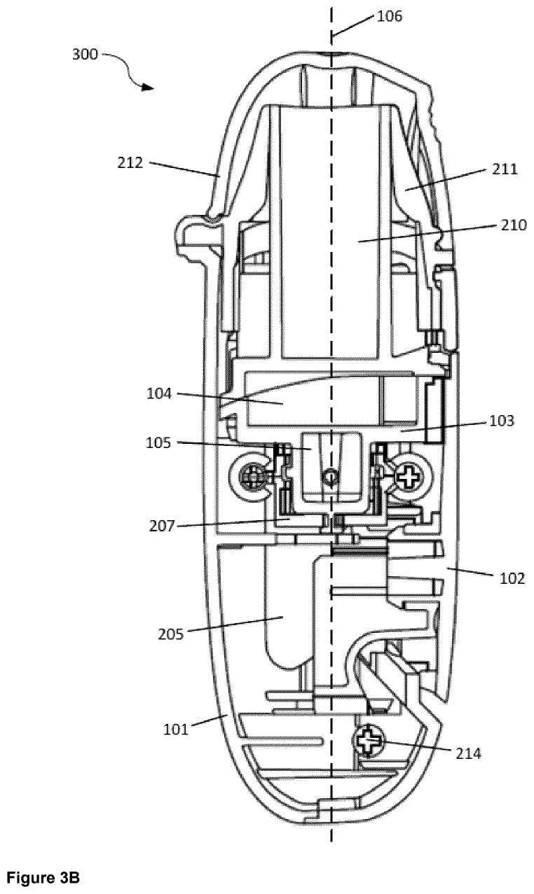

FIG. 3A shows a top view of an inhaler 300 with an open drawer in accordance with the present disclosure. FIG. 3B shows a cross-sectional side view of an inhaler 300 in accordance with the present disclosure. The inhaler 300 may be the same as the inhaler 100 from FIG. 1 and the inhaler 200 from FIG. 2.

Referring firstly to FIG. 3A, the spin chamber 103 is shown in greater detail. As has been discussed, the spin chamber 103 comprises a primary recess 104 and a secondary recess 105. The secondary recess 105 is configured to receive the capsule 213.

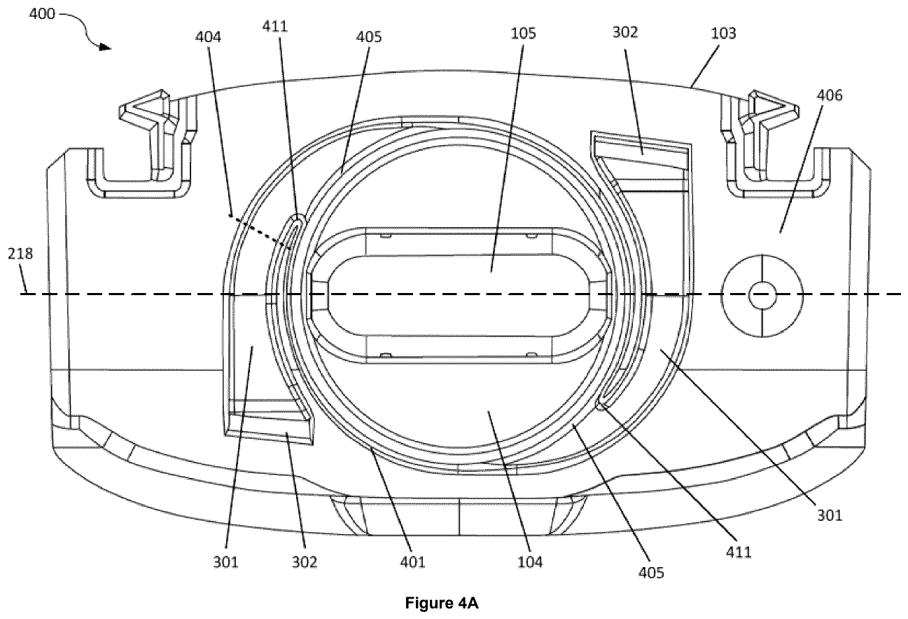

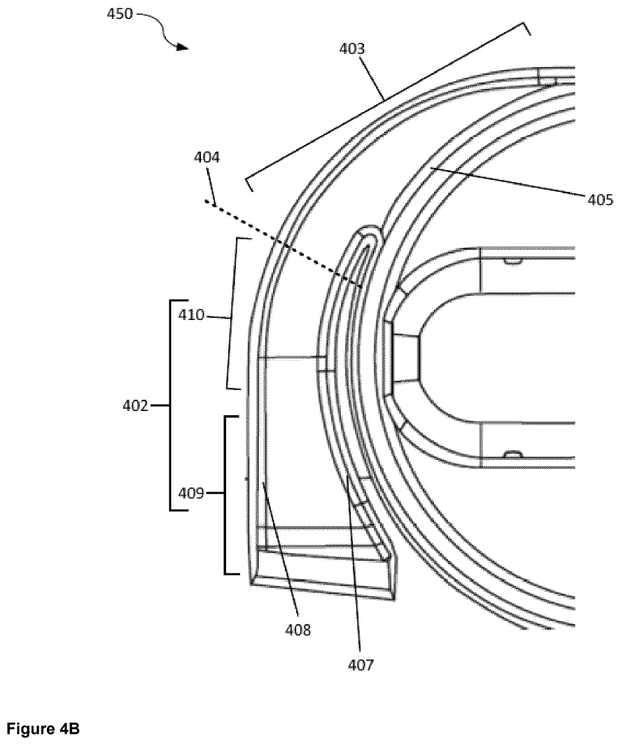

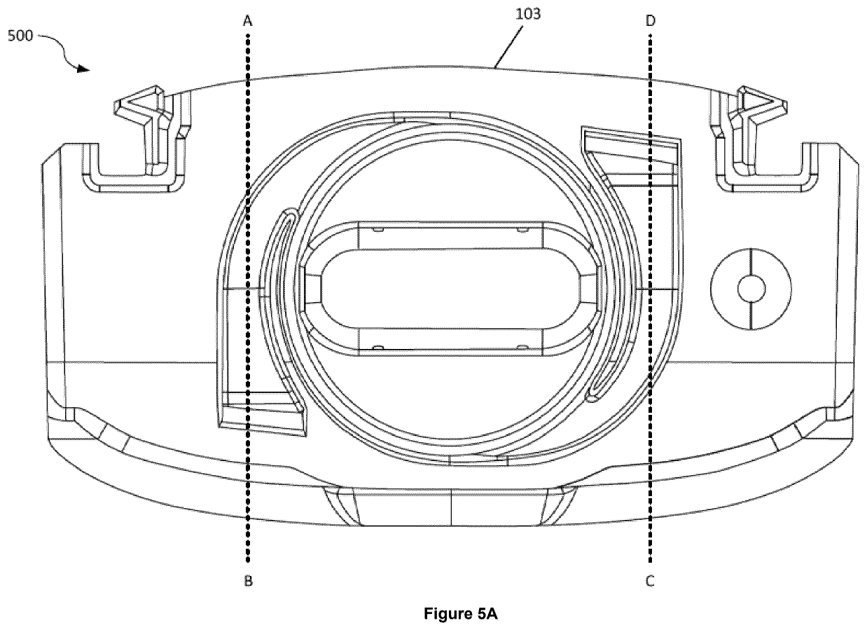

The spin chamber 103 may also comprise at least one curved channel 301, through which air may travel from at least one air inlet 302 into the primary recess 104. The air may then mix with the contents of the capsule 213 during inhalation. FIG. 3A shows an embodiment in which the spin chamber 103 comprises two curved channels 301. The curved channels 301 may be separated from the primary recess 104 along a majority of their length by a curved wall. Features of the curved channels 301 will be described in greater detail with reference to FIGS. 4A-B and 5A-C.

Referring now to FIG. 3B, the internal structure of the inhaler 300 when in the closed position is shown. As has been described with reference to FIG. 2, the components of the drawer 102 are enclosed within the main body 101 when the drawer 102 is in the closed position. The inhalation chimney 210 may extend through the mouthpiece 211, which itself is covered by the cap 212. The spin chamber 103 is positioned at the top of the drawer 102, such that when the drawer 102 is in the closed position, the spin chamber 103 is directly underneath the inhalation chimney 210 with respect to the longitudinal axis 106. The spin chamber 103 is coupled to the supporting framework 205, which is attached to the main body 101 by way of a hinge mechanism 214. The cam post 207 is coupled to the side of the spin chamber 103 and is able to move inwards with respect to the transverse axis, but not upwards or downwards with respect to the longitudinal axis 106.

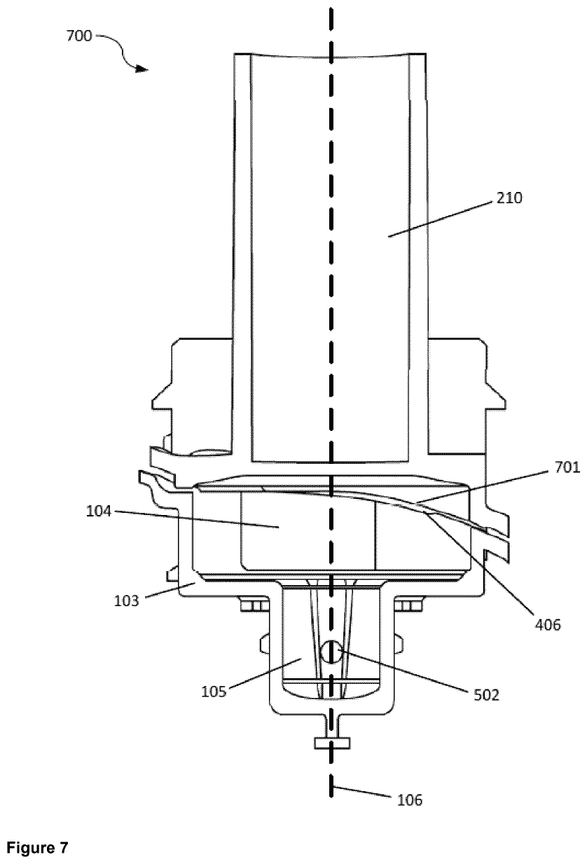

Referring now to both FIGS. 3A and 3B, as has been described, when the drawer 102 is closed into the main body 101 from an open position towards a closed position, the perforating means are configured to interact with a portion of the main body 101, which causes the perforating means to move from a resting position to a perforating position, where the perforating means may perforate a capsule held in the secondary recess 105. As the drawer 102 continues to move towards the closed position, the perforating means return to the resting position and the inhalation chimney 210 is pulled downwards with respect to the longitudinal axis 106, such that a chamber may be defined. As will be described with reference to FIG. 7, this chamber may comprise the primary recess 104, the secondary recess 105 and a volume defined by the inhalation chimney 210. During inhalation, air may travel through the air inlets 302, along the curved channels 301 and into the primary recess 104, into which the capsule 213 has been lifted and the contents of the capsule have begun to empty. The air may then mix with the contents of the capsule 213 as the capsule 213 is spun around by the air.

FIG. 4A shows a top view 400 of a spin chamber 103 of an inhaler, such as the inhaler 100. The spin chamber 103 may be the same spin chamber from FIG. 1. FIG. 4B also shows a top view 450 of the spin chamber 103, but zoomed in further. Both of FIGS. 4A-B therefore show top views of the same spin chamber 103 and will therefore be discussed in tandem.

As with the inhaler 100, the spin chamber 103 may have a longitudinal axis 106 extending from a top of the spin chamber 103 to the bottom of the spin chamber 103. The spin chamber may comprise a top surface 406 that faces upwards with respect to the longitudinal axis 106. The spin chamber 103 may also comprise a primary recess 104 and a secondary recess 105. The primary recess 104 may extend downwards with respect to the longitudinal axis 106 from the top surface 406 of the spin chamber 103. The secondary recess 105 may be located within a bottom surface of the primary recess 104 and may also extend downwards with respect to the longitudinal axis 106. As such, the secondary recess 105 can be considered as an extension of the primary recess 104.

The primary recess 104 may be substantially cylindrical in shape and the secondary recess 105 may be substantially obround in shape. The primary recess 104 has a larger volume than the secondary recess 105. The primary recess 104 may be located substantially near the centre of the top surface 406 with respect to the transverse axis 218.

The primary recess 104 may comprise a curved wall 401 that extends around a majority of the primary recess in a substantially circular configuration. The curved wall 401 may substantially enclose the primary recess 104.

The spin chamber 103 also comprises at least-one curved inlet channel 301, through which air may travel from at least one air inlet 302 on an exterior of the spin chamber 103 into the primary recess 104. The at least one curved inlet channel 301 may be separated from the primary recess 104 along a majority of its length by the curved wall 401 of the primary recess 104. The at least one curved inlet channel 301 may define a curved recess extending downwards from the top surface 406 of the spin chamber. The bottom surface of the curved inlet channel 301 may be substantially flat along a majority of its length. In this way, a bottom surface of the at least one curved inlet channel 301 may be substantially level with the bottom surface of the primary recess 104.

The at least one curved inlet channel 301 may comprise a tangential section 402 and a funnel section 403. The two sections may be separated by a boundary 404. The boundary 404 may be a point along the channel 301, as shown in FIG. 4A, but may instead be a small region of the channel 301 rather than a specific point. The tangential section 402 may be connected at a first end to the air inlet 302 on the exterior surface of the spin chamber 103 and at a second end to the boundary 404. The funnel section 403 may be connected at a first end to the boundary 404 and at a second end to an entry point 405 through which air may enter the primary recess 104 from the channel 301. The entry point 405 may be a wide opening in the curved wall 401 and may be located near to an end of the secondary recess 105, meaning that, in use, the entry point 405 is located near an end of a capsule. In the embodiment shown in FIG. 4A, the boundary 404 is a point, meaning that the second end of the tangential section 402 is connected to the first end of the funnel section 403. The two sections are arranged such that the funnel section 403 is downstream from the tangential section 402.

At least a portion of the tangential section 402 may be substantially tangential to the curved wall 401 of the primary recess 104. The tangential section 402 may comprise a first section 409 and a second section 410. The first section 409 of the tangential section 402 may extend from the first end of the tangential section 402 to a point between the first end of the tangential section 402 and the second end of the tangential section 402. This point may be located approximately half way along a length of the tangential section 402, although it may be located closer to the second end of the tangential section 402 than the first end of the tangential section 402. Alternatively, this point may be located closer to the first end of the tangential section 402 than the second end of the tangential section 402.

The second portion 410 of the tangential section 402 may extend from the point between the first and second ends of the tangential section 402 to the second end of the tangential section 402. The two portions are arranged such that the second portion 410 is downstream from the first portion 409.

The first portion 409 may be widest near the air inlet 302, which, as described above, is located at the first end of the channel 301. A width of the first portion 409 may then decrease with distance downstream, which equates to distance along the channel 301 from the air inlet 302 towards the entry point 405. The second portion 410 may have a substantially uniform width.

The at least one curved inlet channel 301 may comprise an inner wall 407 and an outer wall 408. Both walls may face inwards with respect to the channel 301 and as such may directly face one another. The inner wall 407 may be located closer to the centre of the spin chamber 103 than the outer wall 408. The inner wall 407 may be curved and may substantially follow an outline of the primary recess 104. Since the shape of the primary recess 104 is defined by the curved wall 401, the curvature of the inner wall 407 may substantially match the curvature of the curved wall 401. The inner wall may extend from the air inlet 302, along the entirety of the tangential section 402 and along at least a portion of the funnel section 403 until it reaches the entry point 405. At the entry point 405, the inner wall 407 combines with the curved wall 401 at an edge 411.

The outer wall 408 may be substantially straight In the first portion 409 of the tangential section 402 of the channel 301. In the second portion 410 of the tangential section 402, the outer wall 408 may begin to curve inwards towards the primary recess 104, such that it substantially matches a curvature of the inner wall 407 and the curved wall 401 of the primary recess 104. In the funnel section 403 of the at least one curved inlet channel 301, the outer wall 408 may continue to curve in towards the primary recess 104. The outer wall 408 therefore extends along an entirety of the channel 301. At the entry point 405, the outer wall 408 is substantially tangential to the primary recess 104 and may combine with the curved wall 401 of the primary recess 104. Although the inner wall 407 joins with the curved wall 401 at an edge 411, the tangentiality of the outer wall 408 with the primary recess 104 means that the outer wall 408 joins with the curved wall 401 in a substantially seamless manner. A length of the at least one curved inlet channel 301 may be greater than a radius of the primary recess 104.

As mentioned above, the spin chamber 103 comprises at least one curved inlet channel 301. FIGS. 4A-B show an embodiment in which the at least one curved inlet channel 301 comprises two curved inlet channels 301. This results in a greater air flow through the inhaler, which helps to lift the capsule and allow its contents to mix with the air. The two curved inlet channels 301 may be on opposing sides of the primary recess 104. This means that air may interact with the capsule from both sides, which helps to lift the capsule and allow its contents to mix with the air. A first of the two channels 301 may extend from a first air inlet 302 on a first exterior surface of the spin chamber 103 to a first entry point 405, while a second of the two channels 301 may extend from a second air inlet 302 on a second exterior surface of the spin chamber 103 to a second entry point 405. The second exterior surface may be on an opposite side of the spin chamber 103 to the first exterior surface and the second entry point 405 may be directly opposite the first entry point 405 across the primary recess 104.

The positions of the two curved inlet channels 301 with respect to each other can also be explained geometrically. Taking the top view 400 of the spin chamber 103 to be a planar, two-dimensional grid, and taking the centre of the primary recess 104 to be the centre of the grid, the second of the two channels 301 can be considered to represent substantially a 180-degree rotation of the first of the two channels 301 about the centre of the grid. As such, the tangential sections 402 of each opposing curved inlet channel 301 may be opposite each other across the primary recess 104 and the funnel sections 403 of each opposing curved inlet channel 301 may be opposite each other across the primary recess 104.

The two channels 301 may have different height profiles, which may arise due the curvature of the top surface 406. This will be described in greater detail with respect to FIGS. 5A-C. The first air inlet 302 may have a different height to the second air inlet 302 as a result of the different height profiles of the two channels 301. In order to ensure a balanced air flow through the inhaler, the first air inlet 302 may also have a different width to the second air inlet 302. More specifically, the relative heights and widths of the two air inlets 302 may be chosen such that a cross-sectional area of the first air inlet 302 is substantially equal to a cross-sectional area of the second air inlet 302. This may help to ensure a balanced air flow through both curved inlet channels 301, thus encouraging a stable cyclone to be generated in the spin chamber 103.

As described earlier with reference to FIGS. 1A-B, 2 and 3A-B, the primary recess 104 is configured to allow air to mix with the contents of a capsule, such as the capsule 213 from FIG. 2. The secondary recess 105 is configured to hold therein the capsule and keep it in a position for perforation, such that its contents may be released. The arrangement of the primary recess 104 and the secondary recess 105 allows for the capsule to be lifted out of the secondary recess 105 and up into the primary recess 104 during inhalation. The curved wall 401 of the primary recess 104, which defines the shape of the primary recess 104, encourages rotation of the capsule during inhalation, thus helping to release its contents and allow them to mix with air.

The at least one curved inlet channel 301 is configured to allow air to travel into the primary recess 104, where it may mix with the contents of the capsule during inhalation, as described above.

The inner wall 407 helps to direct the airflow towards the primary recess 104. The extension of the inner wall 407 into the funnel section 403 helps to reduce the migration of any powder (that may have been released from the capsule) from the primary recess 104 back through the channel 301 and into other parts of the inhaler. Such migration is undesirable because it prevents a user from receiving a full dose of antigen binding fragment in the dry powder formulation and may also cause blockages in other parts of the inhaler. The arrangement of the channel 301, and in particular the positions and lengths of its inner wall 407 and outer wall 408, therefore help to eliminate powder spillage through the inlet 302.

The positions and dimensions of the inner wall 407 and the outer wall 408 also define the position and dimensions of the entry point 405. This arrangement means that flow stagnation areas in the channel 301 is eliminated and also helps to ensure that air entering the channel 301 is less susceptible to disruption by surrounding geometry. The location of the entry point 405 also helps to focus air towards the ends of a capsule located in the secondary recess 105, thus helping to lift the capsule and ensure that its contents can mix with the air.

In use, a user of an inhaler comprising the spin chamber 103, such as inhaler 100, will insert a capsule into the secondary recess 105 of the inhaler in its unloaded form and then close the drawer of the inhaler thereby forming a preloaded inhaler. This will perforate the capsule, as has been described with reference to earlier Figures. The user may then inhale through a mouthpiece of the inhaler, causing air to enter the spin chamber 103 through the inlets 302. The air will then travel through the curved inlet channels 301, firstly through the first portion 409 of the tangential section 402, then through the second portion 410 of the tangential section 402, and then through the funnel section 403, before entering the primary recess 104 at the entry point 405. The curved wall 401 of the primary recess causes a vortex to form as the air moves around through the primary recess 104, which lifts the capsule out of the secondary recess 105 and into the primary recess 104. When in the primary recess 104, the capsule is spun around, helping to release its contents, which can then mix with the air. The resulting mixture is then inhaled by the user through the mouthpiece, which functions as an outlet.

FIG. 5A shows a top view 500 of a spin chamber 103 that is identical to the top view 400 from FIG. 4A. The spin chamber 103 may be the same spin chamber 103 from the previous Figures. The top view 500 includes a first line A-B extending along a first of the two curved inlet channels 301 and a second line C-D extending along a second of the two curved inlet channels 301.

FIG. 5B shows a cross-sectional view 510 of the spin chamber 103 along the first line A-B. FIG. 5C shows a cross-sectional view 520 of the spin chamber 103 along the second line C-D. The first line A-B extends through and along a first of the two curved inlet channels 301 and the second line extends through and along a second of the two curved inlet channels 301.

With reference to both FIGS. 5B-C, the cross-sectional view 510 shows the spin chamber 103 comprising at least one curved inlet channel 301. Here, an embodiment shows the spin chamber 103 as comprising two curved inlet channels 301. A first of these channels 301 can be seen in FIG. 5B and a second of these channels 301 can be seen in FIG. 5C. As has been described with reference to previous Figures, each curved inlet channel 301 is connected at a first end to an air inlet 302, which is located on an exterior surface of the spin chamber 103. Each curved inlet channel 301 comprises an inner wall 407 that extends along the channel 301. The curved inlet channels 301 may comprise bottom surfaces that are substantially flat along a majority of their respective lengths.

The spin chamber 103 also comprises at least one guide post 219. FIGS. 5A-C show an embodiment in which the spin chamber comprises two guide posts 219, both located on the same side of the spin chamber 103 but at different ends with respect to the transverse axis 218. The guide posts 219 may extend upwards from a top surface 406 of the spin chamber 103 with respect to the longitudinal axis 106.

The spin chamber 103 may also comprise a pair of small apertures 502, each located on opposing sides of the secondary recess of the spin chamber 103. The spin chamber 103 may also comprise a front bridge 501 that is located on the top surface 406 of the spin chamber 103. The front bridge 501 may be a locking mechanism that extends upwards with respect to the longitudinal axis 106. The front bridge 501 may be positioned along a side of the top surface 406.

The top surface 406 may be curved in a convex manner such that, when the spin chamber 103 is closed inside the inhaler, one side extends higher up the longitudinal axis 106 than the other side. More specifically, the side that is closest to the front casing extends higher up the longitudinal axis 106 than the side that is closest to the rear casing. This can be seen in FIGS. 5B-C, where the side comprising the front bridge 501 extends higher up the longitudinal axis 106 than the side comprising the guide posts 219. The curvature of the top surface 406 enables a drawer containing the spin chamber 103 to be closed into the inhaler via a hinge mechanism, which results in a simplified user experience.

Since the top surface 406 is curved, but the bottom surfaces of the curved inlet channels 301 are substantially flat and level, the depths of the curved inlet channels 301 with respect to the longitudinal axis vary along their lengths. Since the two curved inlet channels 301 have air inlets 302 on opposing sides of the spin chamber 103, the two curved inlet channels 301 have different depth profiles along their lengths. Here, the depth should be interpreted as the distance from the top surface 406 of the spin chamber 103 to the bottom surface of the curved inlet channel 301 with respect to the longitudinal axis 106.

Referring specifically to FIG. 5B, the first of the two curved inlet channels 301 comprises an air inlet 302 located on a side of the spin chamber 103 that extends higher up the longitudinal axis 106 than its opposing side. The first curved inlet channel 301 therefore has its greatest depth at the air inlet 302. Proceeding inwards along the channel 301 (from B to A), the bottom surface of the first curved inlet channel 301 may curve upwards slightly before flattening out. This, combined with the downwards curvature of the top surface 406, provides a distinct depth profile in which the depth of the first curved inlet channel 301 decreases with distance along the channel 301. The first curved inlet channel 301 therefore has a greater depth along the longitudinal axis 106 in its tangential 402 section than in its funnel section 403.

Referring now specifically to FIG. 5C, the second of the two curved inlet channels 301 comprises an air inlet 302 located on a side of the spin chamber 103 that extends lower down the longitudinal axis 106 than its opposing side. The second curved inlet channel 301 therefore has its smallest depth at the air inlet 302. Proceeding inwards along the channel 301 (from D to C), the bottom surface of the first curved inlet channel 301 may curve upwards slightly before flattening out. This, combined with the upwards curvature of the top surface 406, provides a distinct depth profile in which the depth of the first curved inlet channel 301 slightly increases with distance along the channel 301. This depth profile is therefore different from the depth profile of the first curved inlet channel 301. The entry point of each channel 301 may therefore have a different depth with respect to the longitudinal axis 106, despite having a substantially identical width. The second curved inlet channel 301 therefore has a greater depth in its funnel section 403 than in its tangential section 402.

The functions of most of the components in FIGS. 5A-C have already been described with reference to previous Figures. The front bridge 501 is configured to lock onto at least a portion of the inhalation chimney when the inhaler is closed, thus helping to keep the components of the inhaler aligned and sealed during inhalation. The small apertures are configured to allow the needles to pass through them into the secondary recess and perforate the capsule as the drawer is closed into the main body, as has already been described with reference to FIG. 2.

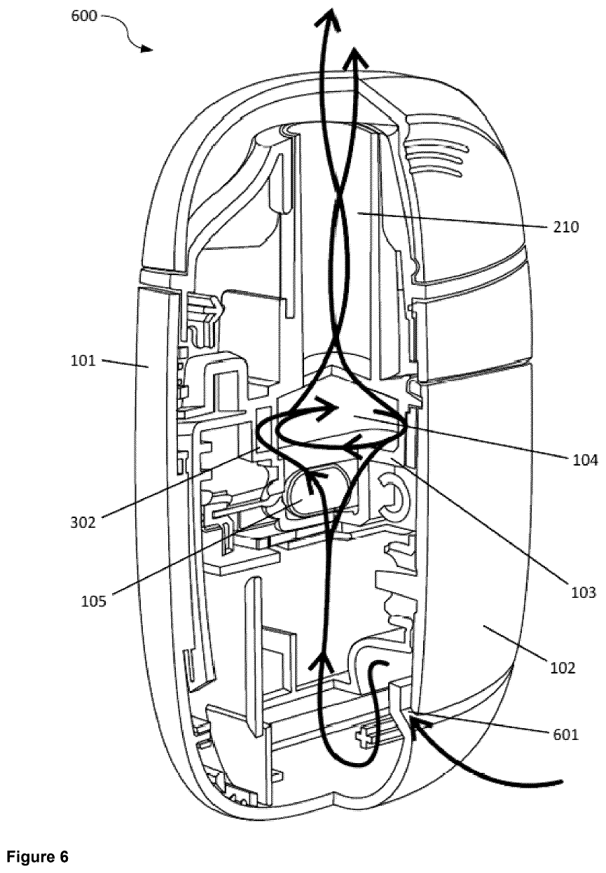

FIG. 6 shows an internal view 600 of air flow through a closed inhaler during inhalation, such as inhaler 100 from FIG. 1. The internal view is semi cross-sectional, as it shows the inhaler with a segment removed to visualise the internal components.

The inhaler may comprise an air inlet 601 of the inhaler through which air may enter the inhaler. FIG. 6 shows this air inlet 601 as being positioned near the bottom of the inhaler with respect to the longitudinal axis 106, specifically underneath the bottom of the drawer 102, but it should be understood that the air inlet 601 may be located at a number of other positions within the inhaler. It should also be understood that multiple air inlets 601 are possible.

The air then travels upwards through the inside of the inhaler as the user inhales. The air enters the spin chamber 103 through the air inlets 302 of the spin chamber 103, where it may pass along the curved inlet channels and into the primary recess 104. The movement of the air through the primary recess 104 causes a perforated capsule to be lifted out of the secondary recess 105 into the primary recess 104, where the air may spin the capsule around. The contents of the capsule can then mix with the air, and this mixture may then travel upwards through the inhalation chimney 210 and into the mouth of the user.

FIG. 7 shows a cross-sectional side view 700 of the inhalation chimney 210 and the spin chamber 103 being held together in accordance with the present disclosure.

When the drawer is in the closed position, the inhalation chimney 210 is positioned directly above the spin chamber 103 with respect to the longitudinal axis 106. The spin chamber 103 may comprise a top surface 406 facing upwards with respect to the longitudinal axis 106. The top surface 406 may also be described as a top surface of the drawer, since the spin chamber 103 is located at the top of the drawer. The top surface 406 of the spin chamber 103 may be curved in a convex manner, as can be seen in FIG. 7.

The inhalation chimney 210 may comprise a bottom surface 701 facing downwards with respect to the longitudinal axis 106. The bottom surface 701 may be curved in concave manner corresponding to the curve of the top surface 406 of the spin chamber 103.

The top surface 406 and the bottom surface 701 are configured to be held together during inhalation, in order to define a chamber within which air can mix with the contents of a capsule inserted into the inhaler. This chamber may comprise the primary recess 104, the secondary recess 105 and a volume defined by the inhalation chimney 210. The curves of the two surfaces correspond to one another so that the spin chamber 103 and the inhalation chimney 210 may enclose the chamber.

When the drawer is in the closed position, the inhalation chimney 210 has been pulled downwards with respect to the longitudinal axis 106, as has been described. There may still be a small gap present between the top surface 406 and the bottom surface 701. As a user inhales through the mouthpiece of the inhaler, the negative pressure caused by the inhalation may cause the spin chamber 103 to move upwards slightly such that the top surface 406 and bottom surface 701 are in direct contact with each other. In this way, a seal may be formed between the two surfaces.

It will be appreciated from the discussion above that the embodiments shown in the Figures are merely exemplary, and include features which may be generalised, removed or replaced as described herein and as set out in the claims.

### Dry Powder Formulations

The dry powder formulations disclosed herein are formulated for pulmonary delivery upon inhalation.

As used herein a “dry powder formulation” refers to a formulation that includes a plurality of solid particles (e.g. microparticles) in a powder composition. The dry powder formulation suitably contains less than 20%, more suitably less than 10%, less than 5%, or less than 3% by weight of moisture. As described herein, dry powder formulations can be utilized for delivery via inhalation to a patient.

The dry powder formulation typically comprises microparticles which comprise the antigen binding fragment, and optionally trileucine and/or leucine. The term “microparticle” as used herein refers to a solid particle having a size mass mean diameter (MMD) of less than 20 μm. Mass mean diameter is a measure of the mean particle size of the microparticles. Mass mean diameter is typically measured by one or more of centrifugal sedimentation, electron microscopy, light scattering or laser diffraction.

As explained in greater detail below, the dry powder formulation may be prepared using spray drying techniques. Such techniques are particularly suitable for preparing microparticles. Thus, the dry powder formulation of the present disclosure may comprise a spray dried formulation and in particular spray dried microparticles. As used herein, the term “spray dried particles” refers to particles manufactured in a process that uses an aerosol phase to spray dry particles to form the basis for dry dosage forms.

The dry powder formulations comprise an antigen binding fragment of an anti-TSLP antibody. Examples of suitable antigen binding fragments that may be used in dry powder formulations described herein include those taught in WO2022/223514, the disclosure of which is incorporated by reference herein. The methods taught in this document may also be used to prepare the antigen binding fragments for use in the dry powder formulations described herein.

### The Sequence of the TSLP Polypeptide is Provided Below:

Met Phe Pro Phe Ala Leu Leu Tyr Val Leu Ser Val Ser Phe Arg Lys Ile Phe Ile Leu Gln Leu Val Gly Leu Val Leu Thr Tyr Asp Phe Thr Asn Cys Asp Phe Glu Lys Ile Lys Ala Ala Tyr Leu Ser Thr Ile Ser Lys Asp Leu Ile Thr Tyr Met Ser Gly Thr Lys Ser Thr Glu Phe Asn Asn Thr Val Ser Cys Ser Asn Arg Pro His Cys Leu Thr Glu Ile Gln Ser Leu Thr Phe Asn Pro Thr Ala Gly Cys Ala Ser Leu Ala Lys Glu Met Phe Ala Met Lys Thr Lys Ala Ala Leu Ala Ile Trp Cys Pro Gly Tyr Ser Glu Thr Gln Ile Asn Ala Thr Gln Ala Met Lys Lys Arg Arg Lys Arg Lys Val Thr Thr Asn Lys Cys Leu Glu Gln Val Ser Gln Leu Gln Gly Leu Trp Arg Arg Phe Asn Arg Pro Leu Leu Lys Gln Gln (SEQ ID NO: 27).

The term “antibody” as used herein refers to a tetrameric glycoprotein that consists of two heavy chains and two light chains, each comprising a variable region and a constant region. “Heavy Chains” and “Light Chains” refer to substantially full-length canonical immunoglobulin light and heavy chains (see e.g., Immunobiology, 5th Edition (Janeway and Travers et al., Eds., 2001). The term “antibody” includes naturally occurring antibodies as well as all recombinant forms of antibodies, e.g., humanized antibodies, fully human antibodies and chimeric antibodies.

The term “antibody fragment” refers to a portion of an intact antibody. The terms “antigen-binding fragment”, “antigen-binding domain”, or “antigen-binding region” of an antibody refers to a portion of an intact antibody that binds to an antigen. Antigen-binding fragments of antibodies include, inter alia, Fab, Fab′, F(ab′)₂, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribady or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Fab” refers to an antibody fragment comprising the VH-CH1 and VL-CL pairing. The term encompasses Fabs comprising non-canonical sequence variants such as amino acid substitutions, deletions, or insertions within the Fab outside of sequence regions typically associated with high sequence variability. For example, Fab variants include Fabs comprising non-canonical amino acid or sequence changes in VH or VL framework regions or in the CH1 or CL domains. Such changes may include the presence of non-canonical cysteines or other derivatizable amino acids, which may be used to conjugate said Fab variants to heterologous moieties. Other such changes include the presence of non-canonical polypeptide linkers, which are polypeptide sequences that covalently bridge between two domains. For example, a Fab variant may comprise a linker polypeptide that covalently attaches the CH1 domain to the VL domain, or the CL domain to the VH domain, such that the Fab can be expressed as a single polypeptide chain.

Sequences of an exemplary Fab of the disclosure (herein termed Fab₁) include:

HCDR1 FAB1
(SEQ ID NO: 1)
Thr Tyr Gly Met His
HCDR2 FAB1
(SEQ ID NO: 2)
Val Ile Trp Tyr Asp Gly Ser Asn Lys His Tyr Ala Asp Ser Val Lys Gly
HCDR3 FAB1
(SEQ ID NO: 3)
Ala Pro Gln Trp Glu Leu Val His Glu Ala Phe Asp Ile
HEAVY CHAIN VH FAB1
(SEQ ID NO: 4)
Gln Met Gln Leu Val Glu Ser Gly Gly Gly Val Val Gln Pro Gly Arg Ser Leu Arg Leu Ser Cys
Ala Ala Ser Gly Phe Thr Phe Arg Thr Tyr Gly Met His Trp Val Arg Gln Ala Pro Gly Lys Gly Leu
Glu Trp Val Ala Val Ile Trp Tyr Asp Gly Ser Asn Lys His Tyr Ala Asp Ser Val Lys Gly Arg Phe
Thr Ile Thr Arg Asp Asn Ser Lys Asn Thr Leu Asn Leu Gln Met Asn Ser Leu Arg Ala Glu Asp
Thr Ala Val Tyr Tyr Cys Ala Arg Ala Pro Gln Trp Glu Leu Val His Glu Ala Phe Asp Ile Trp Gly
Gln Gly Thr Met Val Thr Val Ser Ser
LCDR1 FAB1
(SEQ ID NO: 5)
Gly Gly Asn Asn Leu Gly Ser Lys Ser Val His
LCDR2 FAB1
(SEQ ID NO: 6)
Asp Asp Ser Asp Arg Pro Ser
LCDR3 FAB1
(SEQ ID NO: 7)
Gln Val Trp Asp Ser Ser Ser Asp His Val Val
LIGHT CHAIN VL FAB1
(SEQ ID NO: 8)
Ser Tyr Val Leu Thr Gln Pro Pro Ser Val Ser Val Ala Pro Gly Gln Thr Ala Arg Ile Thr Cys Gly
Gly Asn Asn Leu Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu
FAB1 VARIABLE HEAVY CHAIN VH (nucleic acid)
(SEQ ID NO: 9)
cagatgcagt tggttgaatc tggtggcggc gtggtgcagc ctggcagatc tctgagactg 60
tcttgtgccg cctccggctt caccttcaga acctacggaa tgcactgggt ccgacaggcc 120
cctggcaaag gattggaatg ggtcgccgtg atttggtacg acggctccaa caagcactac 180
gccgactccg tgaagggcag attcaccatc accagagaca actccaagaa caccctgaac 240
ctgcagatga actccctgag agccgaggac accgccgtgt actattgtgc tagagcccct 300
cagtgggaac tcgtgcatga ggcctttgac atctggggcc agggaacaat ggtcaccgtc 360
tcctca 366
FAB1 VARIABLE LIGHT CHAIN VL (nucleic acid)
(SEQ ID NO: 10)
tcatatgttc ttacacaacc accgtcggtt tcggttgctc caggacaaac agctcgaatt 60
acatgcggag gaaacaacct cggatcgaag tcggttcact ggtatcaaca aaagccagga 120
caagctccag ttctcgtggt gtacgatgat tcagatcgac catcatggat cccagagcga 180
ttctcaggat caaactcggg aaatactgcc acgctcacaa tttcacgcgg agaagcggga 240
gatgaagctg attactattg ccaagtgtgg gactcgtcgt cagatcatgt tgttttcgga 300
ggtggaacaa agctcacagt gctc 324
FAB1 HEAVY CHAIN (polypeptide)
(SEQ ID NO: 28)
Gln Met Gln Leu Val Glu Ser Gly Gly Gly Val Val Gln Pro Gly Arg Ser Leu Arg Leu Ser Cys
Ala Ala Ser Gly Phe Thr Phe Arg Thr Tyr Gly Met His Trp Val Arg Gln Ala Pro Gly Lys Gly Leu
Glu Trp Val Ala Val Ile Trp Tyr Asp Gly Ser Asn Lys His Tyr Ala Asp Ser Val Lys Gly Arg Phe
Thr Ile Thr Arg Asp Asn Ser Lys Asn Thr Leu Asn Leu Gln Met Asn Ser Leu Arg Ala Glu Asp
Thr Ala Val Tyr Tyr Cys Ala Arg Ala Pro Gln Trp Glu Leu Val His Glu Ala Phe Asp Ile Trp Gly
Gln Gly Thr Met Val Thr Val Ser Ser Ala Ser Thr Lys Gly Pro Ser Val Phe Pro Leu Ala Pro Ser
Ser Lys Ser Thr Ser Gly Gly Thr Ala Ala Leu Gly Cys Leu Val Lys Asp Tyr Phe Pro Glu Pro
Val Thr Val Ser Trp Asn Ser Gly Ala Leu Thr Ser Gly Val His Thr Phe Pro Ala Val Leu Gln Ser
Ser Gly Leu Tyr Ser Leu Ser Ser Val Val Thr Val Pro Ser Ser Ser Leu Gly Thr Gln Thr Tyr Ile
Cys Asn Val Asn His Lys Pro Ser Asn Thr Lys Val Asp Lys Arg Val Glu Pro Lys Ser Cys Asp
Lys
FAB1 LIGHT CHAIN (polypeptide)
(SEQ ID NO: 29)
Ser Tyr Val Leu Thr Gln Pro Pro Ser Val Ser Val Ala Pro Gly Gln Thr Ala Arg Ile Thr Cys Gly
Gly Asn Asn Leu Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu Gly Gln Pro Lys Ala Ala
Pro Ser Val Thr Leu Phe Pro Pro Ser Ser Glu Glu Leu Gln Ala Asn Lys Ala Thr Leu Val Cys
Leu Ile Ser Asp Phe Tyr Pro Gly Ala Val Thr Val Ala Trp Lys Ala Asp Ser Ser Pro Val Lys Ala
Gly Val Glu Thr Thr Thr Pro Ser Lys Gln Ser Asn Asn Lys Tyr Ala Ala Ser Ser Tyr Leu Ser Leu
Thr Pro Glu Gln Trp Lys Ser His Arg Ser Tyr Ser Cys Gln Val Thr His Glu Gly Ser Thr Val Glu
Lys Thr Val Ala Pro Thr Glu Cys Ser
FAB1 HEAVY CHAIN (nucleic acid)
(SEQ ID NO: 30)
cagatgcagt tggttgaatc tggtggcggc gtggtgcagc ctggcagatc tctgagactg 60
tcttgtgccg cctccggctt caccttcaga acctacggaa tgcactgggt ccgacaggcc 120
cctggcaaag gattggaatg ggtcgccgtg atttggtacg acggctccaa caagcactac 180
gccgactccg tgaagggcag attcaccatc accagagaca actccaagaa caccctgaac 240
ctgcagatga actccctgag agccgaggac accgccgtgt actattgtgc tagagcccct 300
cagtgggaac tcgtgcatga ggcctttgac atctggggcc agggaacaat ggtcaccgtc 360
tcctcagcct ccaccaaggg cccatcggtc ttccccctgg caccctcctc caagagcacc 420
tctgggggca cagcggccct gggctgcctg gtcaaggact acttccccga accggtgacg 480
gtgtcgtgga actcaggcgc cctgaccagc ggcgtgcaca ccttcccggc tgtcctacag 540
tcctcaggac tctactccct cagcagcgtg gtgacagtgc cctccagcag cttgggcacc 600
cagacctaca tctgcaacgt gaatcacaag cccagcaaca ccaaggtgga caagagagtt 660
gagcccaaat cttgtgacaa a 681
FAB1 LIGHT CHAIN (nucleic acid)
(SEQ ID NO: 31)
tcatatgttc ttacacaacc accgtcggtt tcggttgctc caggacaaac agctcgaatt 60
acatgcggag gaaacaacct cggatcgaag tcggttcact ggtatcaaca aaagccagga 120
caagctccag ttctcgtggt gtacgatgat tcagatcgac catcatggat cccagagcga 180
ttctcaggat caaactcggg aaatactgcc acgctcacaa tttcacgcgg agaagcggga 240
gatgaagctg attactattg ccaagtgtgg gactcgtcgt cagatcatgt tgttttcgga 300
ggtggaacaa agctcacagt gctcggtcag cccaaggctg ccccctcggt cactctgttc 360
ccgccctcct ctgaggagct tcaagccaac aaggccacac tggtgtgtct cataagtgac 420
ttctacccgg gagccgtgac agtggcctgg aaggcagata gcagccccgt caaggcggga 480
gtggagacca ccacaccctc caaacaaagc aacaacaagt acgcggccag cagctatctg 540
agcctgacgc ctgagcagtg gaagtcccac agaagctaca gctgccaggt cacgcatgaa 600
gggagcaccg tggagaagac agtggcccct acagaatgtt ca 642

In certain instances, the antigen binding fragment within the dry powder formulation comprises:
a heavy chain variable domain comprising:
   a heavy chain CDR1 sequence comprising the amino acid sequence set forth in SEQ ID NO:1, a heavy chain CDR2 sequence comprising the amino acid sequence set forth in SEQ ID NO:2, and a heavy chain CDR3 sequence comprising the amino acid sequence set forth in SEQ ID NO:3, wherein either of heavy chain CDR1, 2 or 3 optionally comprises a single amino acid substitution, and
a light chain variable domain comprising:
   a light chain CDR1 sequence comprising the amino acid sequence set forth in SEQ ID NO:5, a light chain CDR2 sequence comprising the amino acid sequence set forth in SEQ ID NO:6, and a light chain CDR3 sequence comprising the amino acid sequence set forth in SEQ ID NO:7; wherein either of light chain CDR 1, 2 or 3 optionally comprises a single amino acid substitution.

In certain instances, the antigen binding fragment within the dry powder formulation comprises a heavy chain variable domain comprising a heavy chain CDR1 sequence comprising the amino acid sequence set forth in SEQ ID NO:1, a heavy chain CDR2 sequence comprising the amino acid sequence set forth in SEQ ID NO:2, and a heavy chain CDR3 sequence comprising the amino acid sequence set forth in SEQ ID NO:3, and a light chain variable domain comprising a light chain CDR1 sequence comprising the amino acid sequence set forth in SEQ ID NO:5, a light chain CDR2 sequence comprising the amino acid sequence set forth in SEQ ID NO:6, and a light chain CDR3 sequence comprising the amino acid sequence set forth in SEQ ID NO:7.

For instance, the antigen binding fragment within the dry powder formulation comprises a heavy chain variable domain comprising a heavy chain CDR1 sequence consisting of the amino acid sequence set forth in SEQ ID NO:1, a heavy chain CDR2 sequence consisting of the amino acid sequence set forth in SEQ ID NO:2, and a heavy chain CDR3 sequence consisting of the amino acid sequence set forth in SEQ ID NO:3, and a light chain variable domain comprising a light chain CDR1 sequence consisting of the amino acid sequence set forth in SEQ ID NO:5, a light chain CDR2 sequence consisting of the amino acid sequence set forth in SEQ ID NO:6, and a light chain CDR3 sequence consisting of the amino acid sequence set forth in SEQ ID NO:7.

In additional instances, the antigen binding fragment (a) comprises a VH domain comprising a sequence at least 95%, 90%, 85% or 80% identical to SEQ ID NO: 4 and a VL domain comprising a sequence at least 95%, 90%, 85% or 80% identical to SEQ ID NO: 8; or (b) comprises a VH domain comprising the sequence of SEQ ID NO: 4 and a VL domain comprising the sequence of SEQ ID NO: 8.

In some instances, the antigen binding fragment comprises a heavy chain comprising the sequence set forth in SEQ ID NO: 28 and a light chain comprising the sequence set forth in SEQ ID NO: 29. For instance, the antigen binding fragment may comprise a heavy chain consisting of the sequence set forth in SEQ ID NO: 28 and a light chain consisting of the sequence set forth in SEQ ID NO: 29.

In additional instances, the antigen binding fragment for use in the dry powder formulations comprises (a) a heavy chain variable domain that is a sequence of amino acids that is at least 95%, 90%, 85% or 80% identical to SEQ ID NO: 4; or a sequence of amino acids encoded by a polynucleotide sequence that is at least 80% identical to SEQ ID NO: 30, (b) a light chain variable domain that is a sequence of amino acids that is at least 95%, 90%, 85% or 80% identical to SEQ ID NO: 8; or a sequence of amino acids encoded by a polynucleotide sequence that is at least 80% identical to SEQ ID NO: 31; or a heavy chain variable domain of (a) and a light chain variable domain of (b).

Further light chain CDR (LCDR), light chain variable domain (VL), heavy chain CDR (HCDR) and heavy chain variable domain (VH) sequences of antigen binding fragments of the disclosure include:

LCDR1 FAB2
(SEQ ID NO: 11)
Gly Gly Asn Asn Ile Gly Ser Lys Ser Val His
LIGHT CHAIN VL FAB2
(SEQ ID NO: 12)
Ser Tyr Val Leu Thr Gln Pro Pro Ser Val Ser Val Ala Pro Gly Gln Thr Ala Arg Ile Thr Cys Gly
Gly Asn Asn Ile Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu
LCDR1 FAB3
(SEQ ID NO: 13)
Gly Gly Asn Asn Val Gly Ser Lys Ser Val His
LIGHT CHAIN VL FAB3
(SEQ ID NO: 14)
Ser Tyr Val Leu Thr Gln Pro Pro Ser Val Ser Val Ala Pro Gly Gln Thr Ala Arg Ile Thr Cys Gly
Gly Asn Asn Val Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu
HCDR2 FAB4
(SEQ ID NO: 15)
Val Ile Trp Tyr Asp Gly Ser Asn Lys His Tyr Ala Glu Ser Val Lys Gly
HEAVY CHAIN VH FAB4
(SEQ ID NO: 16)
Gln Met Gln Leu Val Glu Ser Gly Gly Gly Val Val Gln Pro Gly Arg Ser Leu Arg Leu Ser Cys
Ala Ala Ser Gly Phe Thr Phe Arg Thr Tyr Gly Met His Trp Val Arg Gln Ala Pro Gly Lys Gly Leu
Glu Trp Val Ala Val Ile Trp Tyr Asp Gly Ser Asn Lys His Tyr Ala Glu Ser Val Lys Gly Arg Phe
Thr Ile Thr Arg Asp Asn Ser Lys Asn Thr Leu Asn Leu Gln Met Asn Ser Leu Arg Ala Glu Asp
Thr Ala Val Tyr Tyr Cys Ala Arg Ala Pro GIn Trp Glu Leu Val His Glu Ala Phe Asp Ile Trp Gly
Gln Gly Thr Met Val Thr Val Ser Ser
HCDR2 FAB5
(SEQ ID NO: 17)
Val Ile Trp Tyr Asp Gly Ser Asn Lys His Tyr Ala Asp Ser Val Lys Ala
HEAVY CHAIN VH FAB5
(SEQ ID NO: 18)
Gln Met Gln Leu Val Glu Ser Gly Gly Gly Val Val Gln Pro Gly Arg Ser Leu Arg Leu Ser Cys
Ala Ala Ser Gly Phe Thr Phe Arg Thr Tyr Gly Met His Trp Val Arg Gln Ala Pro Gly Lys Gly Leu
Glu Trp Val Ala Val Ile Trp Tyr Asp Gly Ser Asn Lys His Tyr Ala Asp Ser Val Lys Ala Arg Phe
Thr Ile Thr Arg Asp Asn Ser Lys Asn Thr Leu Asn Leu Gln Met Asn Ser Leu Arg Ala Glu Asp
Thr Ala Val Tyr Tyr Cys Ala Arg Ala Pro GIn Trp Glu Leu Val His Glu Ala Phe Asp Ile Trp Gly
Gln Gly Thr Met Val Thr Val Ser Ser
LCDR1 FAB6
(SEQ ID NO: 19)
Gly Gly Gln Asn Leu Gly Ser Lys Ser Val His (SEQ ID NO:19)
LIGHT CHAIN VL FAB6
(SEQ ID NO: 20)
Ser Tyr Val Leu Thr Gln Pro Pro Ser Val Ser Val Ala Pro Gly Gln Thr Ala Arg Ile Thr Cys Gly
Gly Gln Asn Leu Gly Ser Lys Ser Val His Trp Tyr Gin Gin Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu (SEQ ID NO:20)
LCDR1 FAB7
(SEQ ID NO: 21)
Gly Gly Asn Gln Leu Gly Ser Lys Ser Val His
LIGHT CHAIN VL FAB7
(SEQ ID NO: 22)
Ser Tyr Val Leu Thr Gln Pro Pro Ser Val Ser Val Ala Pro Gly Gln Thr Ala Arg Ile Thr Cys Gly
Gly Asn Gln Leu Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Ser Ser Asp His Val Val Phe Gly Gly Gly Thr Lys Leu Thr Val Leu
LCDR3 FAB8
(SEQ ID NO: 23)
Gln Val Trp Asp Thr Ser Ser Asp His Val Val
LIGHT CHAIN VL FAB8
(SEQ ID NO: 24)
Ser Tyr Val Leu Thr Gln Pro Pro Ser Val Ser Val Ala Pro Gly Gln Thr Ala Arg Ile Thr Cys Gly
Gly Asn Asn Leu Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Thr Ser Ser Asp His Val Val Phe Gly Gly Gly Thr Lys Leu Thr Val Leu
LCDR3 FAB9
(SEQ ID NO: 25)
Gln Val Trp Asp Ser Thr Ser Asp His Val Val
LIGHT CHAIN VL FAB9
(SEQ ID NO: 26)
Ser Tyr Val Leu Thr Gln Pro Pro Ser Val Ser Val Ala Pro Gly Gln Thr Ala Arg Ile Thr Cys Gly
Gly Asn Asn Leu Gly Ser Lys Ser Val His Trp Tyr Gln Gln Lys Pro Gly Gln Ala Pro Val Leu Val
Val Tyr Asp Asp Ser Asp Arg Pro Ser Trp Ile Pro Glu Arg Phe Ser Gly Ser Asn Ser Gly Asn
Thr Ala Thr Leu Thr Ile Ser Arg Gly Glu Ala Gly Asp Glu Ala Asp Tyr Tyr Cys Gln Val Trp Asp
Ser Thr Ser Asp His Val Val Phe Gly Gly Gly Thr Lys Leu Thr Val Leu.

In certain instances, the heavy variable chain and the light variable chain domains of the antigen binding fragment for use in the dry powder formulations comprise any of the combinations of CDR sequences set out in the following table:

| | VH CDRs 1, 2 and 3 | VL CDRs 1, 2 and 3 |
|---|---|---|
| Fab₁ | SEQ ID NOs: 1, 2 and 3 | SEQ ID NOs: 5, 6 and 7 |
| Fab₂ | SEQ ID NOs: 1, 2 and 3 | SEQ ID NO: 11, 6 and 7 |
| Fab₃ | SEQ ID NOs: 1, 2 and 3 | SEQ ID NO: 14, 6 and 7 |
| Fab₄ | SEQ ID NO: 1, 15 and 3 | SEQ ID NOs: 5, 6 and 7 |
| Fab₅ | SEQ ID NOs: 1, 17 and 3 | SEQ ID NOs: 5, 6 and 7 |
| Fab₆ | SEQ ID NOs: 1, 2 and 3 | SEQ ID NOs: 19, 6 and 7 |
| Fab₇ | SEQ ID NOs: 1, 2 and 3 | SEQ ID NOs: 19, 6 and 7 |
| Fab₈ | SEQ ID NOs: 1, 2 and 3 | SEQ ID NOs: 5, 6 and 23 |
| Fab₉ | SEQ ID NOs: 1, 2 and 3 | SEQ ID NOs: 5, 6 and 25 |

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, MD (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)) (as shown in the table below).

CDR definitions

| | Kabat | Chothia | IMGT |
|---|---|---|---|
| VH CDR1 | 31-35 | 26-32 | 27-38 |
| VH CDR2 | 50-65 | 52-56 | 56-65 |
| VH CDR3 | 95-102 | 95-102 | 105-117 |
| VL CDR1 | 24-34 | 24-34 | 27-38 |
| VL CDR2 | 50-56 | 50-56 | 56-65 |
| VL CDR3 | 89-97 | 89-97 | 105-117 |

Typically, the antigen binding fragment is a Fab, Fab′, F(ab′)2, scFv, minibody or diabody. In some instances, the antigen binding fragment is a human or humanized Fab. In some instances, the antigen binding fragment is a Fab derived from an IgG1 antibody.

Typically, the antigen binding fragment is present in the dry powder formulation in an amount of from 1% to 60%, or in an amount of from 1% to 45% by weight of the formulation.

In some instances, the antigen binding fragment is present in the dry powder formulation in an amount of from 1% to 5%, such as from 1% to 3% by weight of the formulation. In other instances, the antigen binding fragment is present in the dry powder formulation in an amount of from 5% to 15%, such as from 8% to 12% by weight of the formulation. In other instances, the antigen binding fragment is present in the dry powder formulation in an amount of from 30% to 50%, such as from 35% to 45% by weight of the formulation.

The antigen binding fragment may be present in an amount of 2, 10 or 40% by weight of the formulation.

The dry powder formulations disclosed herein may comprise leucine, trileucine, or a combination thereof. Leucine and/or trileucine have been found to provide stability to the solid particles present in the dry powder formulations, and in particular to provide stability to the antigen binding fragment active agent.

The dry powder formulations may comprise both leucine and trileucine. Dry powder formulations comprising trileucine and leucine are particularly suitable for delivering an antigen binding fragment of an anti-thymic stromal lymphopoietin (TSLP) antibody via inhalation. In this regard, it has been found that the combination of leucine and trileucine in the dry powder formulation allows for a reduction in the overall amount of leucine and trileucine required to desirably stabilize the dry particles (e.g. dry microparticles) compared to dry powder formulations that contain only one of these components. The combination of leucine and trileucine may also contribute to the formulations having increased compressed bulk density in comparison to formulations known in the art, which may enable the delivery of a higher concentration of an active agent to the lungs of a patient following inhalation (due to the ability to include a higher dose of active agent per capsule volume). A further advantage associated with the use of both leucine and trileucine in the formulation is that they contribute to the formulations having increased manufacturing throughput. Such high throughput advantageously greatly impacts the ability to scale up production of the dry powder formulations described herein where large amounts of the formulations are required. In addition to these advantages, a further advantage associated with the use of both leucine and trileucine is that the dry powder formulations have surprisingly been found to form microparticles having a desired size (MMAD), as well as a desired specific surface area (SSA) and roughness, resulting in microparticles that can flow appropriately upon inhalation with minimal undesirable deposition in the throat and on inhaler surfaces.

The term leucine as used herein refers to the amino acid leucine (C₆H₁₃NO₂), which may be a racemic mixture or in either its D- or L-form. In some instances, leucine is used in its L-form. The term leucine may also be used to refer to modified forms of leucine (i.e. where one or more atoms of leucine have been substituted with another atom or functional group). The chemical structure of L-leucine is provided below:

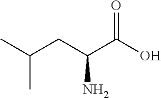

The dry powder formulation may comprise leucine in any suitable amount.

The dry powder formulation typically comprises leucine in an amount of from 1% to 20%, in an amount of from 5% to 15%, or in an amount of 8% to 12%, by weight of the formulation.

The dry powder formulations may also comprise trileucine. The term “trileucine” as used herein refers to the chemical compound in which three leucine molecules are linked together in a peptide, as leucine-leucine-leucine (Leu-Leu-Leu), C₁₈H₃₅N₃O₄. In some instances, the trileucine contains three L-leucine molecules linked together. The chemical structure of trileucine, where three L-leucine molecules have been linked, is provided below:

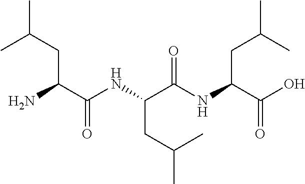 [SMILES: CC(C)C[C@H](NC(=O)[C@H](CC(C)C)NC(=O)[C@@H](N)CC(C)C)C(=O)O]

The dry powder formulations may comprise trileucine in any suitable amount. Typically, the formulations comprise trileucine in an amount of from 1% to 10%, in an amount of from 1% to 5%, or in an amount of from 1% to 3% by weight of the formulation.

Accordingly, in some instances, the formulations comprise leucine in an amount of from 1% to 20% and trileucine in an amount of from 1% to 10% by weight of the formulation. In other instances, the formulations comprise leucine in an amount of from 5% to 15% and trileucine in an amount of from 1% to 5% by weight of the formulation. In other instances, the formulations comprise leucine in an amount of from 8% to 12% and trileucine in an amount of from 1% to 3% by weight of the formulation.

The leucine and trileucine can be included in the dry powder formulations in any suitable mass ratio. Typically, the mass ratio of leucine to trileucine in the formulations is 0.1:1 to 30:1 such as 0.1:1 to 25:1, 0.5:1 to 20:1, 1:1 to 20:1 or 1:1 to 15:1. Optionally, the mass ratio of leucine:trileucine in the formulation is from 1:1 to 12:1 such as 1:1 to 10:1, 1:1 to 7:1, 1:1 to 6:1, or 1:1 to 2:1. Optionally, the mass ratio of leucine:trileucine in the formulation is from 3:1 to about 7:1.

Typically, the dry powder formulation comprises from 1% to 25%, or from 5% to 20%, or from 10% to 15% by weight of a total amount of leucine and trileucine.

Where the dry powder formulations comprise microparticles, in some instances, substantially each of the microparticles of the dry powder formulations comprise leucine and/or trileucine. That is, suitably at least 60% or at least 70% by weight of the microparticles contain leucine and/or trileucine, and more suitably at least 75%, at least 80%, at least 85%, at least 90%, at least 95%, or in some instances 95% to 100% by weight of the microparticles comprise leucine and/or trileucine. In some instances, each of the microparticles of the dry powder formulations comprise leucine and/or trileucine.

In some instances, leucine and/or trileucine can be found in the dry powder formulations, but not contained within or associated with a microparticle of the formulation. Thus, in some instances, free leucine and/or trileucine that is not associated with a microparticle can be found in the dry powder formulations. However, in general, the amount of free leucine and/or trileucine (i.e., not associated with a microparticle) is less than 10%, less than 5%, less than 1%, or less than 0.1% by weight of the total amount of leucine and/or trileucine in the formulations.

Typically, the dry powder formulations further comprise a glass stabilization agent.

The glass stabilizing agent is included to aid in stabilizing the formulation, and in particular, in stabilising the active agent. A “glass stabilisation agent” refers to an excipient that stabilizes an active agent (suitably a polypeptide) in a dry powder formulation, suitably by substituting for water at the active agent surface during drying, or otherwise impeding the degradation process, and forms an amorphous solid that includes the active agent. Examples of glass stabilization agents that may be used in the dry powder formulations disclosed herein include amorphous saccharides, polymeric sugars, buffers, salts, or synthetic polymers (e.g., poly-L-glycolic acid), as well as mixtures of such components. Typically, the glass stabilizing agent comprises an amorphous saccharide, a buffer, or a combination thereof. The glass stabilization agent may comprise both a buffer and an amorphous saccharide.

Where the glass stabilization agent comprises an amorphous saccharide, the amorphous saccharide typically comprises trehalose, sucrose, raffinose, inulin, dextran, mannitol, cyclodextrin, or a combination thereof. In some instances, the amorphous saccharide comprises trehalose.

Where the glass stabilization agent comprises a buffer, the buffer typically comprises a citrate buffer, a phosphate buffer, a histidine buffer, a glycine buffer, an acetate buffer, a tartrate buffer, or a combination thereof. In some instances, the buffer comprises a histidine buffer.

The term “histidine” whether present as a single amino acid or as an amino acid component of a peptide, refers to the amino acid histidine (C₆H₉N₃O₂), which may be a racemic mixture or in either its D- or L-form. In some instances, histidine is used in its L-form. The term histidine may also refer to modified forms of histidine (i.e., where one or more atoms of histidine have been substituted with another atom or functional group). The chemical structure of L-histidine is provided below.

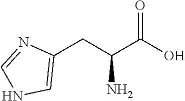 [SMILES: N[C@@H](Cc1c[nH]cn1)C(=O)O]

A histidine buffer typically comprises histidine and a salt thereof, such as the hydrochloride salt of histidine.

It has been surprisingly found by the present inventors that the use of a histidine buffer in the dry powder formulation of the present disclosure may significantly reduce protein aggregation and device and throat deposition of the formulation upon inhalation, at least partly by reducing the level of subvisible particles, compared with other buffers.

The glass stabilization agents may be present in the dry powder formulation in any suitable amount, such as from 45% to 90%, from 70% to 90%, or from 80% to 90% by weight of the formulation.

Where present, the buffer may be present in the dry powder formulation in an amount of from 1% to 20%, from 1% to 10%, or from 1% to 5% by weight of the formulation; such as from 2.5% to 3.5% by weight of the formulation.

Where present, the amorphous saccharide may be present in the dry powder formulation in an amount of from 40% to 90% by weight of the formulation.

Accordingly, where both a buffer and amorphous saccharide are used, the dry powder formulation may comprise the buffer in an amount of from 1% to 10% and the amorphous saccharide in an amount of from 40% to 90%, or the buffer in an amount of from 1% to 5% and the amorphous saccharide in an amount of from 40% to 90%, or the buffer in an amount of from 2.5% to 3.5% and the amorphous saccharide in an amount of from 40% to 90% by weight of the formulation. In these instances, typically, the buffer comprises a histidine buffer and the amorphous saccharide comprises trehalose.

Generally, the dry powder formulation will not comprise a surfactant such as a polysorbate, e.g. polysorbate-80 or polysorbate-20. In some instances, the formulation does not comprise a surfactant and comprise a histidine buffer as described above.

Whilst addition of a surfactant desirably minimises the formation of protein aggregates, it may increase deposition of the dry powder formulation within the inhaler, thus reducing the quantity of the anti-TSLP antibody fragment that is deposited in the lungs. The inclusion of a surfactant may also reduce yield where the formulation is manufactured by spray drying. The use of a histidine buffer in the formulations has, surprisingly, been found to minimise protein aggregation without requiring the inclusion of a surfactant in the formulations.

In some instances, the dry powder formulation comprises from 8% to 12% of leucine; from 1% to 3% of trileucine; from 1% to 5% of a histidine buffer; from 1% to 5% of the antigen binding fragment; and from 75% to 85% of trehalose, by weight of the formulation. For instance, the formulation may comprise 10.5% of leucine; 2% of trileucine; 0.55% of L-histidine; 2.59% of L-histidine HCl; 2% of the antigen binding fragment; and 82.36% of trehalose, by weight of the formulation.

In other instances, the formulation comprises from 8% to 12% of leucine; from 1% to 3% of trileucine; from 1% to 5% of a histidine buffer; from 5% to 15% of the antigen binding fragment; and from 65% to 80% of trehalose, by weight of the formulation. For instance, the formulation may comprise 10.5% of leucine; 2% of trileucine; 0.55% of L-histidine; 2.59% of L-histidine HCl; 10% of the antigen binding fragment; and 74.36% trehalose, by weight of the formulation.

In other instances, the formulation comprises from 8% to 12% of leucine; from 1% to 3% of trileucine; from 1% to 5% of a histidine buffer; from 30% to 50% of the antigen binding fragment; and from 40% to 50% of trehalose, by weight of the formulation. For instance, the formulation may comprise 10.5% of leucine; 2% of trileucine; 0.55% of L-histidine; 2.59% of L-histidine HCl; 40% of the antigen binding fragment; and 44.36% trehalose, by weight of the formulation.

Typically, the dry powder formulations have a compressed bulk density of at least 0.4 g/cm³ and suitably from about 0.4 g/cm³ to about 1.0 g/cm³, and more suitably about 0.4-0.9 g/cm³, about 0.4-0.8 g/cm³, about 0.5-0.8 g/cm³, or about 0.6-0.8 g/cm³, such as about 0.4 g/cm³, about 0.5 g/cm³, about 0.6 g/cm³, about 0.7 g/cm³, or about 0.8 g/cm³.

It has been found by the present inventors that a higher compressed bulk density for the dry powder formulation is desirable since this allows a higher concentration of active agent in the formulation per unit volume. Accordingly, more active agent can be included in each capsule for use with the inhaler.

“Compressed bulk density” refers to the mass per unit volume (suitably g/cm³) of a powder. Suitably, the compressed bulk density (CBD) of the powders may be determined using a density analyzer, such as a GeoPyc® Model 1360 density analyzer (Micromeritics, Norcross, GA). Powder samples are suitably prepared in a low humidity environment (<5% RH), before transfer into the density analyzer sample chamber that has been purged with nitrogen gas. The net weight of the powder sample is recorded, and then a compression force of 10-14N, suitably 12N, is applied to the sample by a plunger, at a rate of 250-350 consolidation steps per second, suitably 300 consolidation steps per second. The linear distance travelled by the plunger for each consolidation step is translated into a volume displacement of the powder sample. An average of the measurements from each consolidation step is then transformed into a calculated bulk density value for the dry powder formulation, expressed in g/cm³.

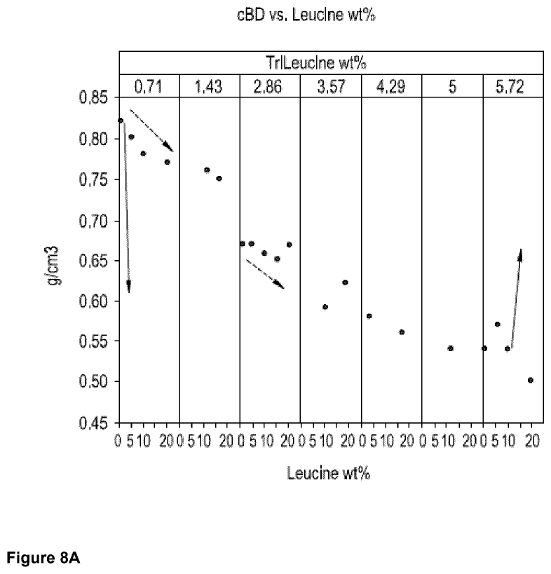

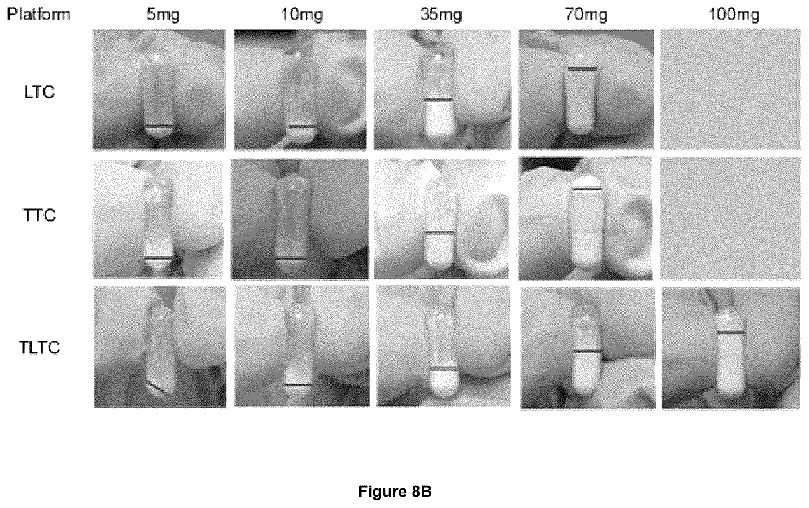

FIG. 8A shows the results of compressed bulk density as a function of leucine and trileucine in the dry powder formulations described herein. Each of the columns represents an amount of trileucine in the formulations. Within each column, the amount of leucine is increased from about 1% to about 20%. As shown, increasing the amount of trileucine results in a lower compressed bulk density, and increasing leucine within each group also reduces the compressed bulk density. To achieve a compressed bulk density of between about 0.5 g/cm³ to about 0.8 g/cm³ the amount of trileucine should be maintained at below 4% by weight. As shown illustratively in FIG. 8B, the combination of leucine and trileucine can result in a dry powder formulation that has a higher bulk density and therefore, for the same amount of fill weight, takes up substantially less volume.

Exemplary platform formulations shown in FIG. 8B are provided below:

Exemplary Platform Formulations

| Platform | % Tre | % Leu | % TLeu | % Cit |
|---|---|---|---|---|
| LTC | 46 | 45 | 0 | 9 |
| TTC | 81 | 0 | 11.2 | 7.8 |
| TLTC | 79 | 10.5 | 2 | 8.5 |

LTC indicates a formulation with no trileucine (TLeu), but containing leucine, trehalose and citrate buffer; TTC indicates a formulation with no leucine (Leu), but containing trileucine, trehalose and citrate buffer; TLTC indicates the inclusion of both leucine and trileucine, as well as trehalose and citrate buffer. Cit refers to citrate buffer. Tre refers to trehalose.

Capsules (size 3 capsules) of each formulation are shown at the respective fill weights in FIG. 8B. As illustrated, for the TLTC formulation, the combination of trileucine and leucine allows for the filling of a capsule with 100 mg of dry powder formulation, while still maintaining some remaining space in the capsule. The other formulations could not be filled above about 70-80 mg fill weight. This represents the dramatic improvement provided by the use of leucine and trileucine in combination to prepare a formulation with a high compressed bulk density, allowing for a high fill weight.

Where the dry powder formulation comprises microparticles, the microparticles that make up the dry powder formulations have a specified mass median aerodynamic diameter (MMAD) when provided in aerosol form. The microparticles also have a specified equivalent optical volume mean diameter (oVMD). oVMD may also be referred to as particle size distribution (PSD or pPSD).

As used herein, “mass median aerodynamic diameter” or “MMAD” is a measure of the aerodynamic size of a dispersed microparticle. The aerodynamic diameter is used to describe an aerosolized powder in terms of its settling behavior and is the diameter of a unit density sphere having the same settling velocity, in air, as the microparticle. The aerodynamic diameter encompasses particle shape, density and physical size of a microparticle. As used herein, MMAD refers to the midpoint or median of the aerodynamic particle size distribution of an aerosolized powder determined by cascade impaction, unless otherwise indicated.

Suitably the microparticles of the dry powder formulations disclosed herein have a mass median aerodynamic diameter (MMAD) of from 1 μm to 10 μm, more suitably 2 μm to 8 μm, 2 μm to 7 μm, 2 μm to 6 μm, 2 μm to 5 μm, or 2 μm to 4 μm.

Suitably, the fine particle fraction (the fraction of particles emitted from an inhalation device having an aerodynamic particle diameter of less than 5 μm) of the dry powder formulations described herein is ≥50%, more suitably ≥60%, even more suitably ≥70%, and most suitably ≥75%. This fine particle fraction (FPF) may contribute to a low device retention of the dry powder formulations of less than 20%, suitably less than 15%, less than 10%, or less than 5%, remaining in a device following delivery to a patient. The fine particle fraction may be determined by cascade impaction, unless otherwise indicated.

In additional instances, the microparticles suitably have an equivalent optical volume mean diameter (oVMD) of from 0.5 μm to 7 μm. Equivalent optical volume mean diameter (oVMD) refers to the mean diameter of a sphere that best approximates a specific optical interaction of the microparticle with light, where half of the microparticles are best approximated by an equivalent sphere smaller, and half of the microparticles are best approximated by an equivalent sphere larger than the mean, when measured using a suitable optical technique.

In exemplary instances, the microparticles have an equivalent optical volume mean diameter (oVMD) of 0.5μ m to 6 μm, 1μ m to 5 μm, 1μ m to 4 μm, 2.5μ m to 4 μm, 2μ m to 4 μm, 2μ m to 3 μm, or 2 μm to 3.5 μm.

As described herein, the use of leucine and trileucine in the dry powder formulations also results in microparticles having the desired sizes (MMAD), as well as desirable specific surface area (SSA) and roughness, resulting in microparticles that can flow appropriately and be delivered to the lungs.

Specific surface area (SSA) of the microparticles is defined as the total surface area of the microparticles per unit of mass (suitably with units of m²/g). Methods of measuring SSA are known in the art, and include for example Brunauer-Emmett-Teller (BET) measurements using specific surface area evaluation of materials by nitrogen adsorption measured as a function of relative pressure. The surface area is determined by calculating the amount of adsorbate gas corresponding to a monomolecular layer on the surface of the microparticles. The technique measures external area and any pore area evaluations to determine the total specific surface area. Instruments for measuring BET are known in the art.

In instances, the specific surface area (SSA) of the microparticles of the dry powder formulations is from 3 m²/g to 8 m²/g. In suitable instances, the SSA of the plurality of microparticles is about 3.5 m²/g to 7.5 m²/g, 4 m²/g to 7 m²/g, 4.5 m²/g to 7 m²/g, 5 m²/g to 7 m²/g, or about 4.5 m²/g-6 m²/g.

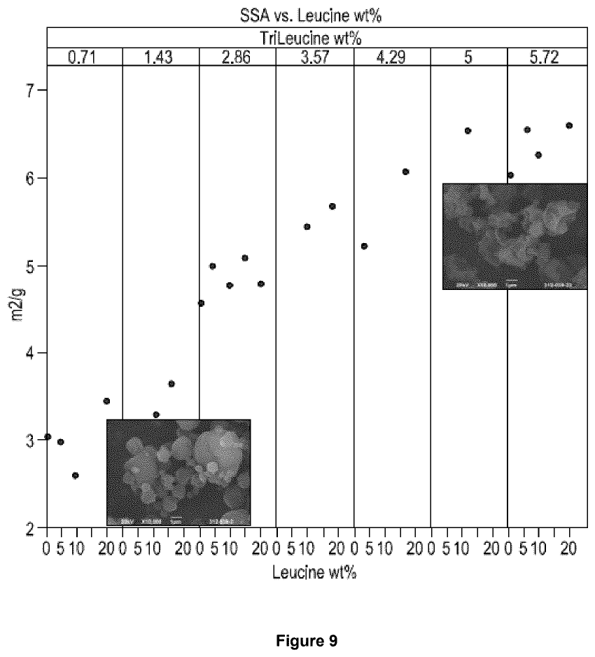

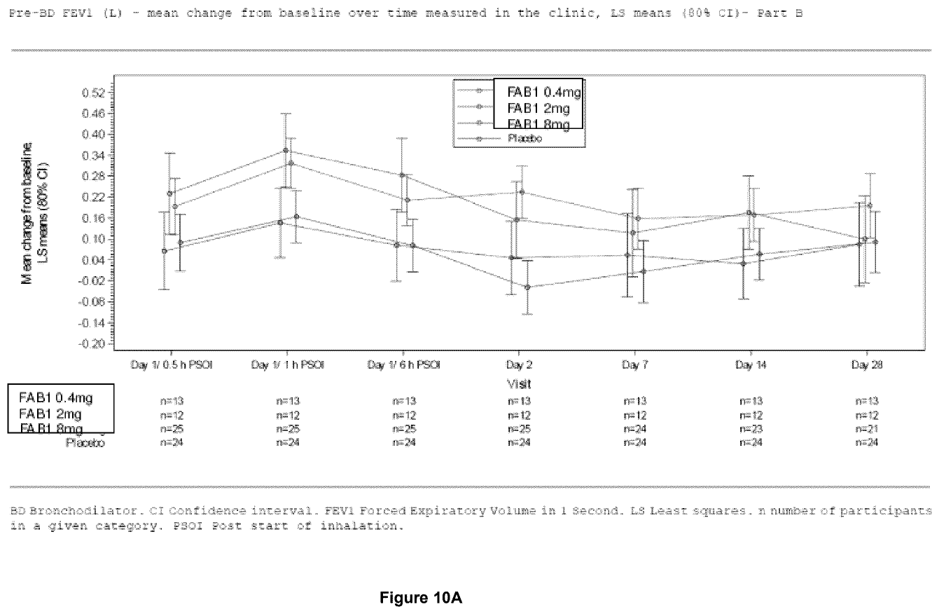

FIG. 9 shows the results of specific surface area measured using BET, in m²/g. Each column within the figure represents a different amount of trileucine in the formulations. Within each column, the amount of leucine increases from about 1% to about 20%. Inset micrographs demonstrate the physical appearance of the microparticles at low SSA (bottom left) and higher SSA (top right). As shown, at lower wt % trileucine, SSA remains below approximately 5 m²/g, but increases with increasing leucine. Above about 2% trileucine, the SSA increases to greater than 3.0 m²/g, and also increases with increasing percent leucine. SSA values above 5.5 m²/g, and approaching 7.0 m²/g, are achieved with trileucine amounts above about 4%. A desirable range of specific surface area of about 4-7 m²/g can readily be achieved using between about 1-6% trileucine, and amounts of leucine between about 1-20%. As shown, by utilizing an amount of trileucine below about 6%, the amount of leucine can be kept below 10%, even below 5%, and still maintain a desirable SSA and microparticles with a surface roughness. The micrograph at the top left shows the shape of microparticles of the dry powder formulations described herein, exhibiting a desirable size, specific surface area, and surface roughness.

Typically, in the dry powder formulations, (i) following reconstitution, the number of sub-visible particles between 5 μm to 200 μm is less than 2.5×10⁴/ml, or less than 0.5×10⁴/ml; (ii) following reconstitution, the number of sub-visible particles between 10 μm to 200 μm is less than 1×10⁴/ml, or less than about 0.2×10⁴/ml; or (iii) following reconstitution, the number of sub-visible particles between 25 μm to 200 μm is less than about 2×10³/ml, or less than about 0.2×10³/ml.

A “sub-visible particle” (“SVP”) as referred to herein is a particle not visible to the naked eye of from about 1 μm to about 200 μm. Removing or reducing the formation of SVPs simplifies the analytical characterization of the formulation, as it removes the burden of tracking the formation of SVPs during manufacturing. The analytical characterization of SVPs may involve the development of orthogonal techniques to identify and quantify SVPs for quality control purposes. Thus, removing SVPs or reducing them to acceptable levels removes the necessity of this characterization step from the manufacturing process, streamlining manufacturing. The removal of SVPs may also make dose ranging more predictable, since the kinetics of drug-release from SVPs is unknown. Furthermore, removing SVPs is likely to increase the amount of active agent available to engage in pharmacological activity post-reconstitution, which may mean not only that a higher delivered dose can be achieved, but a more accurate prediction of the delivered dose can be calculated. A higher delivered dose may also benefit the patient, for example, by potentially reducing the number or frequency of doses that must be delivered for extracting a pharmacological benefit.

The presence of sub-visible particles can be determined by reconstituting a dry powder formulation and the liquid having a cloudy quality. The actual determination of the presence of SVPs can be confirmed using a technique like dynamic flow imaging microscopy, such as microflow imaging (MFI). In MFI (which is also known as flow imaging microscopy (FIM) or dynamic imaging analysis (DIA)), bright-field images are captured in successive frames as a continuous sample stream passes through a flow-cell positioned in the field of view of a microscopic system. The digital images of the particles present in the sample are processed by image morphology analysis software that allows their quantification in size and count. MFI is an established technique for subvisible particle analysis. Dynamic flow imaging microscopy combines microfluidic flow microscopy and high-resolution imaging particle analysis to quantify SVP counts. MFI can bin these counts across a particle size range, for example, by binning particles counts in a size range of about 1 to about 200 μm, about 2 μm to about 200 μm, about 5 μm to about 200 μm, about 10 μm to about 200 μm and about 25 μm to about 200 μm. An alternative technique for the measurement of SVP is background membrane imaging (BMI). Briefly, SVPs from a liquid sample are isolated onto a filer surface for counting analysis by a microscope. The BMI software images the baseline prior to particle isolation and, then subtracts that baseline pixel-by-pixel from the isolated particles so that only photographic information remains (Vargas et al., 2020).

In certain instances, following reconstitution, the number of SVPs of between 5 μm to 200 μm in size is less than about 30,000 particles per ml, such as 25,000 particles per ml, 20,000 particles per ml, 15,000 particles per ml, 10,000 particles per ml or 5,000 particles per ml. In certain instances, the number of SVPs of 5 μm to 200 μm in size are less than 1,000 particles per ml. In certain instances, the number of SVPs of between 5 μm to 200 μm in size are less than below 1,000 particles per ml. In certain instances, the number of SVPs of between 5 μm to about 200 μm in size are less than below 100 particles per ml.

In certain instances, following reconstitution, the number of SVPs of 10 μm to 200 μm in size are less than about 100,000 particles per ml, such as 90,000 particles per ml, 80,000 particles per ml, 70,000 particles per ml, 60,000 particles per ml, 50,000 particles per ml, 40,000 particles per ml or 30,000 particles per ml. In certain instances, the number of SVPs of 10 μm to 200 μm in size are less than about 10,000 particles per ml. In certain instances, the number of SVPs of 10 μm to 200 μm in size are less than about 2,000 particles per ml. In certain instances, the number of SVPs of 10 μm to about 200 μm in size are less than 100 particles per ml.

In certain instances, following reconstitution, the number of SVPs of 25 μm to 200 μm in size is less than about 200,000 particles per ml, such as 180,000 particles per ml, 170,000 particles per ml, 160,000 particles per ml, 150,000 particles per ml or 140,000 particles per ml. In certain instances, the number of SVPs of about 5 μm to about 200 μm in size are less than about 50,000 particles per ml. In certain instances, the number of SVPs of 5 μm to 200 μm in size are less than about 10,000 particles per ml. In certain instances, the number of SVPs of about 5 μm to about 200 μm in size are less than about 2,000 particles per ml. In certain instances, the number of SVPs of 10 μm to about 200 μm in size are less than about 200 particles per ml.

In some instances, the number of SVPs is determined following reconstitution in water, to an antigen-binding fragment concentration of either 2.5 mg/ml or 30 mg/ml.

### Capsules

The dry powder formulation is administered from capsules using the inhalers of the present disclosure. A suitable capsule may be obround-shaped with a length that is greater than its width. The capsule may have a length and width that are each from 2% to 30%, such as from 5% to 20%, shorter than the length and width of the secondary recess in the inhaler.

The capsules comprise a capsule shell which contains the dry powder formulation. The capsule shell may comprise cellulose or a derivative thereof, or other suitable materials known in the art. In some instances, the capsule shell comprises hydroxypropylmethyl cellulose.

The capsule may comprise any suitable volume of the dry powder formulation. Suitably, the capsule may comprise from 10 mg to 30 mg, or 15 mg to 25 mg, such as 20 mg, of the dry powder formulation.

Suitably, the capsule comprises from 0.1 mg to 10 mg of the antigen binding fragment of an anti-TSLP antibody, such as from 0.4 mg to 8 mg of the antigen binding fragment. In some instances, the capsule comprises 0.4 mg, 2 mg or 8 mg of the antigen binding fragment of an anti-TSLP antibody.

### Processes for Manufacturing Dry Powder Formulations

The dry powder formulations for use in the inhalers of the disclosure can be manufactured using any suitable technique known in the art, such as spray drying or other suitable drying techniques.

Typically, the dry powder formulations disclosed herein are produced by spray drying. For example, the following spray drying process may be used for manufacturing the dry powder formulations disclosed herein:
a. Providing an aqueous solution of pH 5 to pH 6 comprising an antigen binding fragment of an anti-TSLP antibody;
b. Spray drying the aqueous solution of (a) to produce dry powder particles; and
c. Collecting the dry powder particles;

Typically, the aqueous solution comprises leucine, trileucine, or a combination thereof as discussed above.

The aqueous solution is prepared by combining its constituents in an aqueous solvent to create a solution in which each of the components is dissolved. Temperature control may be added as desired or required to increase the solubility of the various components to form the aqueous solution. Exemplary liquid solvents include water, including deionized water, as well as dilute solutions of alcohols with water. In instances, the active agent is suitably added to the aqueous solution after the addition and dissolution of the remaining components of the feedstock.

The aqueous solution may be filtered prior to spray drying. In certain instances, the liquid feedstock may be filtered through a 0.22 micron filter. In certain instances, the aqueous solution is filtered prior to the addition of the active agent. In certain instances, the aqueous solution is filtered after the addition of the active agent prior to spray drying.

The spray drying step of the process typically comprises atomizing the aqueous solution.

Atomizing refers to converting the solution to fine droplets, suitably using a pressurized gas (such as an inert gas or compressed dry air). Exemplary devices for producing an atomized solution are known in the art and include the use of various atomizing nozzles have desired sizes and flow characteristics. Exemplary parameters for the atomizing including an outlet temperature of 50° C.-90° C., suitably 60° C.-80° C., or about 70° C.; a feedstock feed rate of about 8-15 ml/min, suitably about 9-14 ml/min, about 10-13 ml/min, or about 12 ml/min; an atomizer gas flow rate of about 9-15 kg/hour (hr. or h), suitably about 10-14 kg/hr, about 12-14 kg/hr, or about 13 kg/hr; and drying gas flow rate of about 60-100 kg/hr, suitably about 60-90 kg/hr, about 70-90 kg/hr, or about 80 kg/hr.

After atomizing the aqueous solution, the atomized solution may then be dried, suitably under heat and in combination with flowing air to aid in the drying. The result of the drying yields a plurality of microparticles. Drying temperatures typically range from about 50°−100° C., or about 60°−100° C., or about 70°−90° C.; air flow rate can be on the order of about 10-40 m³/hour.

The atomized droplets contain the dissolved components, initially as a liquid droplet. As the droplet dries, different components of the formulation begin to saturate and precipitate at varying rates. A shell begins to form around an outer surface of the microparticles of the dry powder formulations. This shell suitably includes the leucine or trileucine (or both if included) components at an outer surface of the shell. It should be noted that leucine and trileucine become preferentially located at an outer surface of the microparticles, while smaller amounts of leucine and trileucine can also be found throughout the microparticles. In instances, a higher concentration of leucine and trileucine are suitably found at or near the surface of the microparticles, rather than near the center of the microparticles. In instances, the center of particles contains a substantial amount of the active agent, along with other excipient components as described herein, suitably in an amorphous form. As used herein, a “substantial amount” of the active agent means at least about 60% of the active agent (i.e., of the total active agent in the formulation) is located at or near the center of the microparticles, suitably at least about 70%, and more suitably at least about 75%, at least about 80%, at least about 85%, at least about 90%, at least about 95%, and in instances about 95%-100%, of the active agent is located at or near the center of the microparticles.

Accordingly, typically, the particles contain leucine, trileucine or a combination thereof located substantially throughout the microparticles, but with higher amounts at or near the surface of the microparticles. As used herein “substantially throughout the microparticles” means that the leucine and/or trileucine are located in a gradient from the outer surface of the microparticles toward the center of the microparticles, but suitably with decreasing amounts of the leucine and/or trileucine as you move toward the center, and in instances, no leucine or trileucine are found at the center of the microparticles where the active agent is located. In other instances, the amounts of leucine and/or trileucine can be substantially uniform throughout a cross-section of the microparticles.

### Methods of Treatment and Medical Uses

Disclosed herein are methods of administering dry powder formulations, methods of treatment and medical uses as described above. In all of the methods and uses, the dry powder formulation is administered from a capsule using a preloaded inhaler which comprises the capsule. The preloaded inhaler and capsule are as described herein. The dry powder formulation is administered by inhalation, such as by oral inhalation.

Within this section of the description any reference to a method of treatment shall be construed to disclose the corresponding instances of uses or formulations for use.

“Treat” or “treatment” refer to both therapeutic treatment and prophylactic or preventative measures, wherein the object is to prevent or slow down (lessen) an undesired physiological change or disorder. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Therapeutically effective amount” refers to an amount of an antigen binding fragment of an anti-TSLP antibody disclosed herein or other drug effective to “treat” a disease or disorder in a subject or mammal.

“Subject” means any subject, particularly a mammalian subject, for whom diagnosis, prognosis, or therapy is desired, except where the subject is defined as a ‘healthy subject’. Mammalian subjects include humans; domestic animals; farm animals; such as dogs, cats, guinea pigs, rabbits, rats, mice, horses, cattle, cows, and so on. Preferably the subject is human. The patient may be an adult, or a child or adolescent.

Medical conditions that can be treated using the methods described herein include those that effect the nervous system, the endocrine system, the muscular system, the cardiovascular system, the digestive system, the respiratory system (and specifically the lungs), hormone systems, the immune system, the reproductive system, etc.

Typically, the condition to be treated is a TSLP-related condition in a subject in need thereof. Typically, the TSLP-related condition is a TSLP-related inflammatory condition such as asthma, sepsis, septic shock, atopic dermatitis, allergic rhinitis, allergic rhinosinusitis, allergic conjunctivitis, eosinophilic esophagitis, rheumatoid arthritis, chronic obstructive pulmonary disease (COPD), asthma, COPD overlap syndrome (ACOS), chronic bronchitis, emphysema, chronic rhinosinusitis with or without nasal polyps, vasculitis, GvHD, uveitis, chronic idiopathic urticaria, sinusitis or pancreatitis.

In some instances, the TSLP-related condition is asthma, COPD, allergic rhinitis, allergic rhinosinusitis, allergic conjunctivitis, eosinophilic esophagitis, chronic spontaneous urticaria or chronic rhinosinusitis.

In instances where the TSLP-related condition is asthma, the asthma is typically mild asthma, moderate asthma, severe asthma, eosinophilic asthma, non-eosinophilic asthma, or low eosinophilic asthma, high eosinophilic asthma or no eosinophilic asthma.

In some instances, the subject has a documented history of asthma, such as the types of asthma discussed above, of at least one year.

Asthma is a chronic inflammatory disease of the airways affecting 1-18% of the population in different countries and is characterized by bronchial hyperreactivity and reversible airflow limitation. It is defined by the history of respiratory symptoms such as wheeze, shortness of breath, chest tightness and cough. The etiology of asthma is thought to be multi-factorial and there are recognizable clusters of demographic, clinical and/or pathophysiological phenotypes. In patients with more severe phenotypes, some phenotype-guided treatments are available. However, no strong relationship between pathological symptoms and clinical presentations and response to therapies have been established.

Asthma may be diagnosed or assessed by a number of different measures, including:

Airway inflammation evaluated using a standardized single-breath Fraction of Exhaled Nitric Oxide (FeNO) (ATS, Am J Respir Crit Care Med. 171(8):912-30, 2005). FeNO has not been established for confirming asthma diagnosis but elevated FeNO has been associated with asthma characterized by a Type 2 airway inflammation.

Determining the atopic status. This can be identified by a skin prick test with common environmental allergies or by measuring the level of specific IgE in serum. As with FeNO, allergy tests do not rule in or rule out a diagnosis of asthma but the presence of atopy increases the probability that a patient with respiratory symptoms has allergic asthma.

Bronchial provocation testing. These tests monitor variable airflow limitation to assess airway hyperresponsiveness (AHR). Subjects can be challenged with chemical agents such as methacholine. Such tests are moderately sensitive to the diagnosis of asthma.

The term “FENO” refers to fractional exhaled nitric oxide, which is a biomarker for bronchial or airway inflammation. FENO is produced by airway epithelial cells in response to inflammatory cytokines, such as TSLP, IL-4 and IL-13. FENO levels in healthy adults range from 2 to 30 parts per billion (ppb). An exemplary assay for measuring FENO comprises subjects inhaling to total lung capacity through the NIOX MINO® Airway Inflammation Monitor and then exhaling for 10 seconds at 50 ml/sec (assisted by visual and auditory cues).

Different asthma subtypes have been identified, including allergic asthma, non-allergic asthma, late-onset asthma (which typically tends to be non-allergic), asthma with persistent airflow limitation (which is linked to airway wall remodeling, leading to a long-standing, persistent, irreversible airflow limitation), and asthma with obesity (which is typically linked to a non/low eosinophilic mechanism of action). In some instances, subjects treated by the present disclosure may have any type or origin of asthma.

There are different levels of asthma severity, which are currently assessed retrospectively from the level of treatment required to control symptoms and exacerbations. The severity index comprises three main groups: mild asthma, moderate asthma, and severe asthma. Severity of asthma is defined on the GINA scale by the level of treatment required to gain adequate control of symptoms. The GINA scale is defined in the “Pocket Guide for Asthma Management and Prevention,” Global Initiative for Asthma; 2019. Unless otherwise stated herein, references to “moderate asthma” or “severe asthma” are in accordance with the definitions on the GINA scale. For instance, moderate asthma refers to asthma that has a Global Initiative for Asthma (GINA) scale of 3 or less, suitably a GINA scale of 2 or 3 (i.e. GINA step 2 or step 3), and severe asthma refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

In some instances, the subject has a history of ≥1 or ≥2 severe exacerbation(s) within the last 12 months prior to the treatment. Severe exacerbations are defined as those episodes that lead to hospitalisation, emergency room visit, and/or treatment with oral glucocorticosteroid as detailed below:

Inpatient hospitalization: an admission to an inpatient facility and/or evaluation and treatment in healthcare facility for ≥24 hours due to asthma;

Emergency room or urgent care visit: evaluation and treatment for <24 hours in an emergency department or urgent care centre due to asthma required systemic corticosteroids; and/or Use of a temporary bolus/burst of systemic corticosteroids (or a temporary increase in stable OCS background dose) for at least 3 consecutive days to treat symptoms of asthma worsening; a single depo-injectable dose of corticosteroids will be considered equivalent to a 3-day bolus/burst of systemic corticosteroids.

In some instances, the asthma is moderate asthma, severe asthma, or moderate-to-severe asthma. In some instances, the asthma is not well-controlled on controller or reliever standard of care therapies defined in steps 1 and 2 of the GINA scale. In some instances, therefore the asthma to be treated by the present disclosure may be uncontrolled asthma. In some instances, the asthma is moderate asthma uncontrolled on standard of care therapy, severe asthma uncontrolled on standard of care therapy, or moderate-to-severe asthma uncontrolled on standard of care therapy. Standard of care (SOC) therapy is as defined in the GINA scale.

In some instances, the subject has a baseline blood eosinophil count of ≥150 cells/μl or ≥300 cells/μl. In some instances, baseline refers to the blood eosinophil count prior to commencement of the treatment, e.g. within one month of the commencement of treatment.

In certain instances, the methods disclosed herein improve lung function in a subject with asthma. In some instances, the improving lung function means one or more of the following: improvement compared to baseline of pre-bronchodilator (BD) FVC, post-BD-FVC, pre-BD-FEV1, post-BD FEV1, mean morning PEF or mean evening PEF.

In some instances, the improvement means achieving the minimal clinical important difference (MCID) for each of pre-bronchodilator (BD) FVC, post-BD-FVC, pre-BD-FEV1, post-BD FEV1, mean morning PEF or mean evening PEF, respectively.

The phrase “minimal clinically important difference” or “MCID” means that smallest change in a treatment outcome that an individual patient would identify as important and which would indicate a change in the patient's management. Some MCIDs are experimentally validated and others are study specific.

The term “pre-BD FEV1”, “preBD FEV1” or “pre-bronchodilator (BD) FEV1” refers to pre-bronchodilator forced expiratory volume 1. This is a measurement of forced expiratory volume of a subject in 1 second before administration of bronchodilator. In some instances, the minimum clinical important difference for pre-BD-FEV1 is 100 ml.

In some instances, the increase in pre-BD-FEV1 compared to baseline (e.g. the pre-BD-FEV1 value prior to commencement of treatment) is at least 5 ml, at least 10 ml, at least 15 ml, at least 20 ml, at least 25 ml, at least 30 ml, at least 35 ml, at least 40 ml, at least 45 ml, at least 50 ml, at least 55 ml, at least 60 ml, at least 65 ml, at least 70 ml, at least 75 ml, at least 80 ml, at least 85 ml, at least 90 ml, at least 95 ml, at least 100 ml, at least 105 ml, at least 110 ml, at least 115 ml, at least 120 ml, at least 125 ml, at least 130 ml, at least 135 ml, at least 140 ml, at least 145 ml, at least 150 ml, at least 160 ml, at least 170 ml, at least 180 ml, at least 190 ml, at least 200 ml, at least 210 ml, at least 220 ml, at least 230 ml, at least 240 ml or at least 250 ml. In some instances, the increase in pre-BD-FEV1 compared to baseline (e.g. the pre-BD-FEV1 value prior to commencement of treatment) is at least 80 ml at day 2 after commencement of treatment, at least 45 ml or at least 100 ml at day 7 after commencement of treatment, at least 100 ml at day 14 after commencement of treatment, or at least 5 ml or at least 100 ml at day 28 after commencement of treatment.

The term “postBD-FEV1” or “post-bronchodilator (BD) FEV1” refers to post-bronchodilator forced expiratory volume 1. This is a measurement of forced expiratory volume of a subject in 1 second after administration of bronchodilator.

The term “pre-BD-FVC” or “pre-bronchodilator (BD) forced vital capacity (FVC)” refers to bronchodilator Forced vital capacity. This is the total amount of air exhaled by a subject during a forced expiratory volume test or FVC test before administration of bronchodilator.

The term “post-BD-FVC” or “post-bronchodilator (BD)-FVC” refers to post-bronchodilator forced vital capacity. This is the total amount of air exhaled by a subject during a forced expiratory volume test or FVC test after administration of bronchodilator.

The term “bronchodilator” is a substance which dilates the bronchi and bronchioles, decreasing resistance in the respiratory airways and increasing airflow to the lungs. Suitable bronchodilators include a short-acting beta agonist (SABA) such as albuterol (90 1-1 g metered dose) or salbutamol (1 00 1-1 g metered dose) or equivalent (Sorkness et al, J Appl Physiol. 1 04(2):394-403, 2008).

The term “peak expiratory flow rate (PEF)” indicates the fastest rate that a subject can force air out of the lungs during a forced expiratory volume test or FVC test, typically measured in Litres/minute.

The term “forced expiratory volume (FEV)” is the amount of air expired by a subject during the first, second, and third seconds of the FVC test. The term FEV1 as explained above is the amount of air expired by a subject during the first second of the FVC test.

The term “forced vital capacity” (FVC) or the forced vital capacity test is a measurement of the total amount of air exhaled forcefully and quickly by a subject after inhaling as much as possible.

In some instances, pre-BD-FEV1, post-BD-FEV1, pre-BD-FVC, post-BD-FVC Spirometry is performed according to ATS/European Respiratory Society (ERS) guidelines (Miller et al, Eur Respir J. 26(1):153-61, 2005). For example, multiple forced expiratory efforts (at least 3 but no more than 8) is performed at each spirometry session and the 2 best efforts that meet ATS/ERS acceptability and reproducibility criteria are recorded. The best efforts will be based on the highest FEV1. The maximum fluvial exhalation volume (FEV1) of the 2 best efforts will be used for the analysis. Both the absolute measurement (for FEV1 and forced vital capacity (FVC)) and the percentage of predicted normal value will be recorded using appropriate reference values. The highest FVC will be reported regardless of the effort in which it occurred (even if the effort did not result in the highest FEV1).

Post-bronchodilator (Post-BD) spirometry testing is assessed after the subject has performed pre-BD spirometry. Pre-BD FEV1 is measured as defined above using spirometry before administration of a suitable bronchodilator to the subject. To measure post-BD FEV1, maximal bronchodilation is induced using a short-acting beta agonist (SABA) such as albuterol (90 1-1 g metered dose) or salbutamol (1 00 1-1 g metered dose) or equivalent with a spacer device for a maximum of 8 total puffs (Sorkness et al, J Appl Physiol. 1 04(2):394-403, 2008). The highest pre- and post-BD FEV1 obtained after 4, 6, or 8 puffs is used to determine reversibility and for analysis. Reversibility algorithm is as follows: % Reversibility=(post-BD FEV1-pre-BD FEV1)×1 00/pre-BD FEV1.

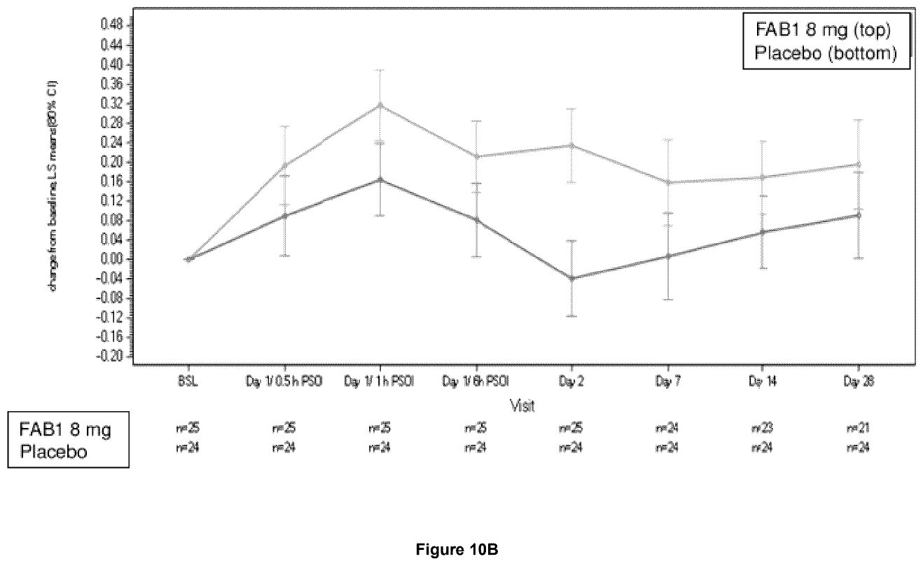

The Ph1b portion of the study Investigating the Safety, Tolerability and Effects of FAB1 in Healthy Subjects and Subjects with Asthma on Inhaled Corticosteroids and Long-acting Beta-agonists (NCT05110976) demonstrated that FAB1 resulted in numerical improvements in lung function (namely pre-BD FEV1), as shown in FIG. 10. Accordingly, in some instances, the methods disclosed herein improves pre-BD-FEV1 in a subject with asthma.

In some instances, the improvement in lung function is within 0.5 h of first dose with the formulation disclosed herein. In some instances, the improvement in lung function is within 1 h of first dose with the formulation disclosed herein. In some instances, the improvement in lung function is within 6 h of first dose with the formulation disclosed herein. In some instances, the improvement in lung function is within 24 h of first dose with the formulation disclosed herein. In some instances, the improvement in lung function is within 7 days of first dose with the formulation disclosed herein. In some instances, the improvement in lung function is within 14 days of first dose with the formulation disclosed herein. In some instances, the improvement in lung function is within 28 days of first dose with the formulation disclosed herein. FIG. 10B shows that subjects in part B of NCT05110976 who were administered 8 mg FAB1 displayed a trend towards an improvement in pre-BD FEV1 in the high dose arm, with 105 ml increase at day 28, and early effects seen at 6 hours after first dose.

In some instances, the methods disclosed herein improve symptoms of asthma in a subject. In some instances, improving symptoms of asthma means one or more of the following: improvement compared to baseline mean asthma symptom diary score, ACQ-6 score, AQLQ score, or SGRQ score.

### Asthma Control Questionnaire (ACQ) 6

The Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV1 (Juniper et al, October 1999). The ACQ-6 is a shortened version of the ACQ that omits the FEV1 measurement from the original ACQ score. Questions are weighted equally and scored from 0 (totally controlled) to 6 (severely uncontrolled). The mean ACQ score is the mean of the responses. Mean scores of 0.75 indicate well-controlled asthma, scores between 0.75 and 1.5 indicate partly-controlled asthma, and a score >1.5 indicates uncontrolled asthma (Juniper et al, Respir Med. 1 00(4):616-21, 2006). Individual changes of at least 0.5 are considered to be clinically meaningful (Juniper et al, Respir Med. 99(5):553-8, 2005). Accordingly, in some instances, the minimum clinical important difference for ACQ-6 is 0.5.

In some instances, the methods disclosed herein improve lung function in the subject, wherein the method results in a 0.5 point improvement in ACQ-6 score compared to baseline.

### Standardised Asthma Quality of Life Questionnaire for 12 Years and Older (AQLQ(S)+12)

The AQLQ(S)+12 (or “AQLQ”) is a questionnaire that measures the health-related quality of life experienced by asthma subjects. The questionnaire comprises 4 separate domains (symptoms, activity limitations, emotional function, and environmental stimuli). Subjects are asked to recall their experiences during the previous 2 weeks and to score each of the questions on a 7-point scale ranging from 7 (no impairment) to 1 (severe impairment). The overall score is calculated as the mean response to all questions. The 4 individual domain scores (symptoms, activity limitations, emotional function, and environmental stimuli) are the means of the responses to the questions in each of the domains. The responder definition for AQLQ(s)+12 is 0.5-point improvement from baseline. Accordingly, in some instances, the minimum clinical important difference for AQLQ is 0.5.

In some instances, the disclosed methods improve lung function in the subject, wherein the method results in a 0.5 point improvement in AQLQ compared to baseline.

St. George's Respiratory Questionnaire (SGRQ)

The SGRQ is a 50-item PRO instrument developed to measure the health status of patients with airway obstruction diseases (Jones et al 1991). The questionnaire is divided into 2 parts: part 1 consists of 8 items pertaining to the severity of respiratory symptoms in the preceding 4 weeks; part 2 consists of 42 items related to the daily activity and psychosocial impacts of the individual's respiratory condition. The SGRQ yields a total score and 3 domain scores (symptoms, activity, and impacts). The total score indicates the impact of disease on overall health status. This total score is expressed as a percentage of overall impairment, in which 100 represents the worst possible health status and 0 indicates the best possible health status. Likewise, the domain scores range from 0 to 100, with higher scores indicative of greater impairment. Based on empirical data and interviews with patients, a mean change score of 4 units is associated with a minimum clinically important difference (MCID). Specific details on the scoring algorithms are provided by the developer in a user manual (Jones et al 2009). SGRQ is a qualified biomarker and the responder definition is generally a 4 point improvement from baseline. Accordingly, in some instances, the minimum clinical important difference for SGRQ is 4.

In some instances, the disclosed methods improve lung function in a subject, wherein the method results in a 4 point improvement in SGRQ score compared to baseline.

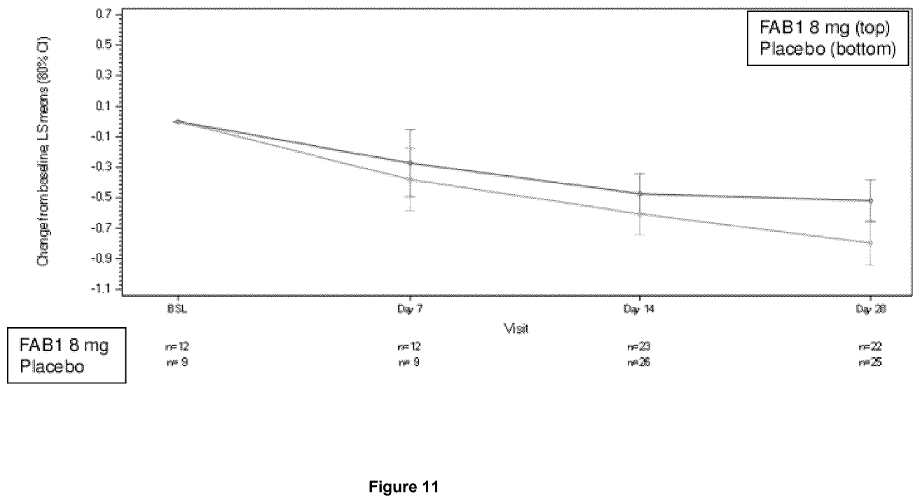

In some instances, the improvement in asthma symptoms is within 7 days of first dose with the formulation disclosed herein. In some instances, the improvement in asthma symptoms is within 14 days of first dose with the formulation disclosed herein. In some instances, the improvement in asthma symptoms is within 28 days of first dose with the formulation disclosed herein. FIG. 11 shows that subjects in part B of NCT05110976 who were administered 8 mg FAB1 displayed a trend towards an improvement in ACQ-6 score as evidenced by mean change from baseline over time in the high dose arm (8 mg).

### Composite Exacerbation (CompEx) Event Rate

The CompEx Asthma is a composite endpoint that allows evaluation of treatment effect on exacerbation involving fewer participants compared with severe exacerbations. There are two main types of CompEx Asthma events:
Severe exacerbations of asthma
Diary-based (objective deterioration)

In some instances, the disclosed methods comprise improving time to a first CompEx event in a subject with asthma.

In another instance, the disclosed methods comprise improving lung function in a subject with asthma, wherein the improvement in lung function comprises an improved in time to first CompEx event compared to placebo.

In some instances, the disclosed methods comprise improving lung function in a subject with asthma, wherein the improvement in lung function comprises an improvement in time to first CompEx event. In some instances, the improved time is compared to placebo. In some instances, the improved time is compared to baseline. In some instances, baseline is the time to first CompEx event in a subject who has not received the treatment described herein.

As described herein, the ability to deliver the antigen binding fragment of the anti-TSLP antibody via inhalation provides a delivery mechanism more amenable to use in a primary care setting.

In instances of the methods of treating asthma, the dry powder formulation is administered frequently and at lower dosages than a systemically administered anti-TSLP medicine. In some instances, the formulation may be administered daily. Such instances may be more convenient for the subject or patient. Furthermore, such instances may reduce side effects that can occur via systemic administration.

In some instances, the anti-drug antibody (ADA) prevalence in the subject following treatment with the dry powder formulation disclosed herein is less than 6%, less than 5% or less than 4% and/or the ADA incidence is less than 4%, less than 3% or less than 2%. ADA prevalence is the percentage of ADA-evaluable participants who were ADA+ at any time, while ADA incidence is the percentage of ADA-evaluable participants who had treatment emergent anti-drug antibodies (TE-ADA+).

In some instances, the formulations provide for the possibility of treating patients with moderate-severe asthma who could be managed in a primary care setting, or for treating patients with moderate-severe asthma with poor access to treatment via specialist care. For example, the formulations may be useful for the treatment of moderate-severe asthma patients with a Global Initiative for Asthma (GINA) scale of 4-5. Suitably, the formulations provide for the possibility of treating moderate-severe asthma that is uncontrolled. Suitably, the formulations provide for the possibility of treating moderate-severe asthma that is uncontrolled on medium dose to high dose ICS:LABA with one or more exacerbations and frequent symptoms.

The dry powder formulation disclosed herein may be administered in combination with any known therapy for asthma, including any agent or combination of agents that are known to be useful, or which have been used or are currently in use, for treatment of inflammatory diseases, e.g. asthma or COPD. Exemplary active agents that can be administered in combination with the dry powder formulation described herein include, but are not limited to, inhaled corticosteroids (ICS), bronchodilators (including long-acting beta agonists (LABA), long-acting anti-muscarinic agonists (LAMA), short-acting beta agonist (SABA), and muscarinic β2-agonists (MABA)), antihistamines, antileukotrienes, PDE-4 inhibitors, janus kinase inhibitors and phosphoinositide 3-kinase inhibitors.

Thus, in some instances, the subject to be or being treated is co-administered a background therapy. In some instances, the subject is already receiving the background therapy prior to the treatment. In some instances, the background therapy is selected from: inhaled corticosteroids, Leukotriene modifiers, long-acting beta agonists (LABAS), long-acting muscarinic antagonists (LAMAs), combination therapies such as Fluticasone and salmeterol, budesonide and formoterol, mometasone and formoterol and fluticasone and vilanterol, theophylline, short-acting beta agonists (SABAs), ipratropium; or a combination of ipratropium and albuterol or ipratropium and oral corticosteroids.

In some instances, the background therapy comprises medium or high dose ICS (as per GINA 2023 report) in combination with LABA (GINA step 4 or 5 therapy).

The term “combination” refers to either a fixed combination in one dosage unit form, or a combined administration where a dry powder formulation as described herein and a combination partner (e.g. another drug, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. The component that is not a dry powder formulation of the present disclosure may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. an anti-TSLP Fab and a combination partner, are both administered to a patient simultaneously in the form of a single entity or dosage. The term “non-fixed combination” means that the therapeutic agents, e.g., an anti-TSLP Fab and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient, provided that the dry powder formulation which comprises the anti-TSLP antibody is administered from a capsule with an inhaler of the present disclosure. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

Typically, the methods of the present disclosure comprise administering the formulation by the inhaler at a dose of from 0.1 mg to 16 mg of the antigen binding fragment per dose, such as from 0.1 mg to 10 mg, 0.2 mg to 10 mg per dose, or from 0.4 mg to 8 mg per dose.

Typically, each capsule is suitable for administering a single dose of antigen binding fragment in the form of a dry powder formulation. A single inhaler may be used to administer multiple doses of the antigen binding fragment, for instance up to 30 doses of the antigen binding fragment, such as from 7 to 21 doses, before it is disposed of. The inhalers of the present disclosure may be used to provide delivery of the antigen binding fragments across multiple doses with minimal reduction in performance.

In some instances, the formulation is administered by the inhaler at a dose of from 0.2 mg to 0.6 mg of the antigen binding fragment per dose; from 1 mg to 3 mg of the antigen binding fragment per dose; or from 6 mg to 10 mg of the antigen binding fragment per dose. In exemplary instances, the formulation is administered by the inhaler at a dose of from 0.4 mg, 2 mg or 8 mg per dose.

The dry powder formulation may be administered by the inhaler to the subject in any suitable amount necessary for treating the TSLP-related condition. In some instances, the dose is administered daily and optionally once daily. In other instances, the dose is administered twice daily or every other day.

In some instances, the dry powder formulation comprising the anti-TSLP antigen binding fragment is administered to a subject for at least 2 weeks, at least one month, at least two months, at least three months, at least four months, at least five months, at least six months, at least seven months, at least eight months, at least nine months, at least ten months, at least eleven months, or at least twelve months.

In some instances, the dry powder formulation comprising the anti-TSLP antigen binding fragment is administered or is to be administered to a subject in need thereof for at least 2 weeks, at least 4 weeks, at least 8 weeks, at least 12 weeks, at least 16 weeks, at least 20 weeks, at least 24 weeks, at least 28 weeks, at least 32 weeks, at least 36 weeks, at least 40 weeks, at least 44 weeks, at least 48 weeks, or at least 52 weeks.

In some instances, the dry powder formulation comprising the anti-TSLP antigen binding fragment is administered or is to be administered to a subject in need thereof for 12 to 52 weeks, e.g. for 12 to 52 weeks.

### Kits

As mentioned above, an inhaler as described herein in its unloaded form is provided as part of a kit. The kit comprises (i) the unloaded inhaler; and (ii) one or more capsules as described herein for loading into the spin chamber of the inhaler. It will be appreciated that the capsules contain a dry powder formulation as described herein which comprises an antigen binding fragment of an anti-TSLP antibody.

The kit may comprise one capsule or more than one capsule. For instance, the kit may comprise from 7 to 28 capsules, such as 7, 14, 21 or 28 capsules.

The disclosed kits will in some instances have labels or package inserts indicating that the associated dry powder formulations are useful for treating a subject suffering from, or predisposed to a disease or disorder, such as the conditions discussed above.

The present disclosure will now be illustrated by the following, non-limiting, examples.

### EXAMPLES

### Example 1—Study 1: TLTC Vs TLTH at pH 5 and 6

### Experimental

Formulations comprising FAB1 were prepared. FAB1 is an antibody fragment directed against the cytokine TSLP and may be used for treatment of moderate to severe asthma patients. This study evaluated two factors of the formulation, namely buffer (citrate vs histidine) and pH (5 and 6).

To test the effect of histidine buffer at different pH on protein formulation and protein aggregation, six formulations were produced as shown in the following table:

Study 1 Batches

| | | FAB1 | Batch | FS |
|---|---|---|---|---|
| Lot number | Formulation | Strength | size (g) | Vol (ml) |
| 21-WS-016 | TLTC, pH 5 | 10% | 10 | 133 |
| 21-WS-017 | TLTC, pH 5 | 40% | | |
| 21-WS-018 | TLTH, pH 6, 5% His | 10% | | |
| 21-WS-019 | TLTH, pH 6, 5% His | 40% | | |
| 21-WS-023 | TLTH, pH 5, 5% His | 10% | | |
| 21-WS-024 | TLTH, pH 5, 5% His | 40% | 20 | 267 |

The composition of each formulation is summarised in the following table:

Study 1 Formulation Targets

| | Component (% w/w) | | | | | |
|---|---|---|---|---|---|---|
| | 10% FAB1, | 40% FAB1, | 10% FAB1, | 40% FAB1, | 10% FAB1, | 40% FAB1, |
| | TLTC, pH 5 | TLTC, pH 5 | TLTH, pH 6 | TLTH, pH 6 | TLTH, pH 5 | TLTH, pH 5 |
| | Lot | | | | | |
| | 21-WS-016 | 21-WS-017 | 21-WS-018 | 21-WS-019 | 21-WS-023 | 21-WS-024 |
| FAB1 | 10.0 | 40.0 | 10.0 | 40.0 | 10.0 | 40.0 |
| Trehalose | 69.55 | 39.55 | 72.70 | 42.70 | 72.05 | 42.05 |
| Leucine | 10.50 | 10.50 | 10.50 | 10.50 | 10.50 | 10.50 |
| Trileucine | 2.00 | 2.00 | 2.00 | 2.00 | 2.00 | 2.00 |
| Trisodium | 5.50 | 5.50 | — | — | — | — |
| citrate | | | | | | |
| Citric acid | 2.45 | 2.45 | — | — | — | — |
| L-Histidine | — | — | 2.00 | 2.00 | 0.35 | 0.35 |
| Histidine-HCl | — | — | 2.80 | 2.80 | 5.10 | 5.10 |

The results are shown in the following table:

Study 1 processing results.

| | | 10% TLTC, | 40% TLTC, | 10% TLTH, | 40% TLTH, | 10% TLTH, | 40% TLTH, |
|---|---|---|---|---|---|---|---|
| | | pH 5 (21- | pH 5 (21- | pH 6 (21- | pH 6 (21- | pH 5 (21- | pH 5 (21- |
| Description | | WS-016) | WS-017) | WS-018) | WS-019) | WS-023) | WS-024) |
| Formulation | Batch size | 10 | 10 | 10 | 10 | 10 | 20 |
| | (g) | | | | | | |
| | Solids | 75 | 75 | 75 | 75 | 75 | 75 |
| | loading | | | | | | |
| | (mg/ml) | | | | | | |
| | Feedstock | 133.3 | 133.3 | 133.3 | 133.3 | 133.3 | 166.6 |
| | (FS) Volume | | | | | | |
| | (ml) | | | | | | |

| | | | | | | |
|---|---|---|---|---|---|---|
| Yield (based on batch size) | 69.6 | 87.4 | 69.3 | 67.7 | 79 | 68.6 |
| (%) | | | | | | |
| Yield (based on FS mass) | 75.0 | 94.2 | 81.5 | 79.6 | 85.3 | 71.1 |
| (%) | | | | | | |
| FS pH | 5.1 | 5.1 | 6.0 | 5.8 | 5.0 | 5.1 |

1) Yield based on batch size is calculated as the net collector yield divided by the nominal batch size.
2) Yield based on FS mass is calculated as the net collector yield divided by the actual FS mass.

The following table shows that all batches were at 10 g batch size except for the 40% TLTH, pH 5, which was 20 g. Despite the small batch sizes, the spray drying yield based on FS mass was high for all of them. The powders were produced at low throughput processing parameters.

Study 1 solid state testing.

| | | 10% FAB1 | | | 40% FAB1 | | |
|---|---|---|---|---|---|---|---|
| | | TLTC | TLTH | TLTH | TLTC | TLTH | TLTH |
| | | pH 5 (21- | pH 6 (21- | pH 5 (21- | pH 5 (21- | pH 6 (21- | pH 5 (21- |
| | | WS-016) | WS-018) | WS-023) | WS-017) | WS-019) | WS-024) |
| Drug Substance | mg/ml | 42.8 | 44.7 | 41.7 | 42.8 | 44.7 | 41.8 |
| (DS) protein | % recovery | 85 | 85 | 84 | 85 | 85 | 84 |
| FS protein | mg/ml | 7.1 | 7.0 | 7.3 | 28.9 | 27.5 | 29.0 |
| | % recovery | 94 | 94 | 97 | 97 | 92 | 97 |
| Bulk powder (BP) | % w/w | 9.4 | 9.7 | 7.6* | 35.5 | 37.0 | 36.4 |
| protein | % recovery | 93.9 | 96.6 | 75.7 | 88.8 | 92.6 | 91.0 |
| DS High | % monomer | 99.9 | 100 | 99.8 | 99.9 | 100 | 99.8 |
| performance size | primary peak | | | | | | |
| exclusion | (MPP) | | | | | | |
| chromatography | % | 0.1 | 0.0 | 0.2 | 0.1 | 0.0 | 0.20 |
| (HP-SEC) | aggregates | | | | | | |
| FS HP-SEC | % MPP | 99.9 | 99.9 | 99.8 | 99.9 | 99.9 | 99.8 |
| | % | 0.1 | 0.1 | 0.2 | 0.1 | 0.1 | 0.2 |
| | aggregates | | | | | | |
| BP HP-SEC | % MPP | 99.9 | 99.9 | 99.7 | 99.9 | 99.9 | 99.7 |
| | % | 0.1 | 0.1 | 0.3 | 0.1 | 0.1 | 0.3 |
| | aggregates | | | | | | |
| Primary particle | d10 (μm) | 0.5 | 0.5 | 0.5 | 0.5 | 0.5 | 0.5 |
| size distribution | d50 (μm) | 1.5 | 1.5 | 1.8 | 1.7 | 1.8 | 1.5 |
| (pPSD) | d90 (μm) | 3.5 | 3.5 | 3.8 | 3.8 | 3.9 | 3.4 |
| | Span | 2.0 | 2.0 | 1.9 | 1.9 | 1.9 | 2.0 |

| | | | | | | |
|---|---|---|---|---|---|---|
| Moisture content (%) | 1.4 | 0.7 | 0.7 | 1.5 | 1.0 | 0.9 |
| Glass transition temperature (Tg) | 94 | 98 | 99 | 97 | 100 | 99 |
| (° C.) | | | | | | |
| Compressed Bulk Density (cBD) | 0.6584 | 0.7245 | 0.7498 | 0.6070 | 0.6479 | 0.6702 |
| (g/cm³) | | | | | | |
| Specific Surface Area (SSA) | 5.8251 | 5.1879 | 4.9904 | 5.5543 | 5.5313 | 5.2441 |
| (m²/g) | | | | | | |

*Lower value than expected and does not align with the FS protein concentration (97% recovery)

It can be seen that the solid properties were similar between all formulations and that protein purity was very high in DS, FS and BP with low aggregation. Primary particle size distribution was similar across all formulations and moisture content was low, being below 1% for the histidine formulations. Glass transition was high and similar for all formulations. High glass transition and low residual moisture content suggest room temperature stable powder. Compressed bulk density was in a favourable range with slightly higher numbers for the TLTH formulations indicating a slightly more flowable powder also reflected in a slightly increased surface area.

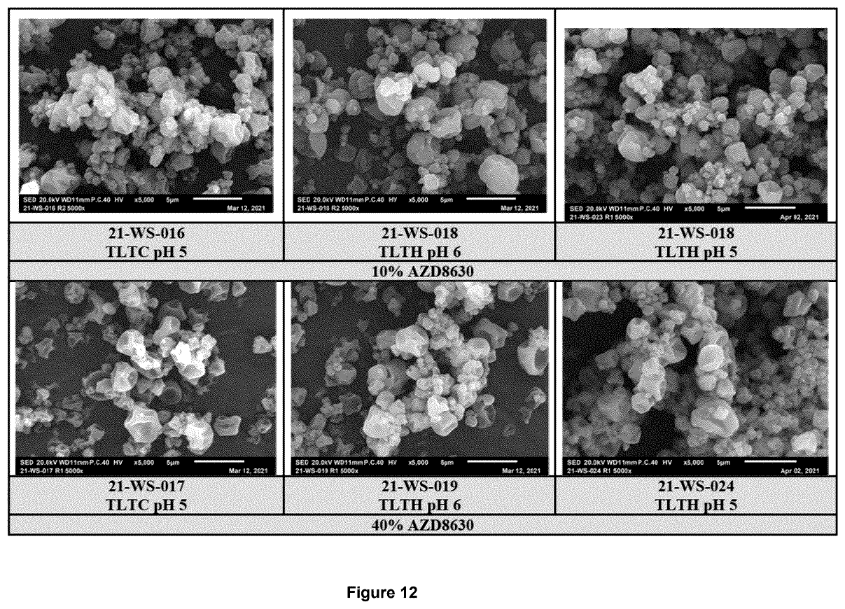

Scanning Electron Microscopy (SEM) analysis of the formulations demonstrated good morphology with similar appearance for both the TLTC and TLTH formulations (FIG. 12).

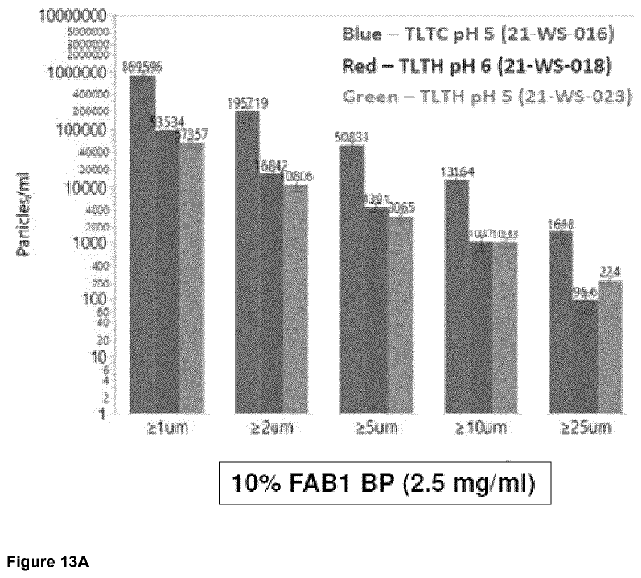

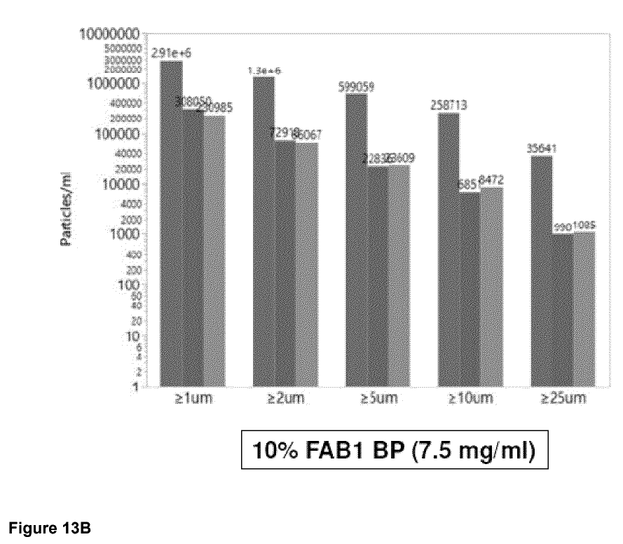

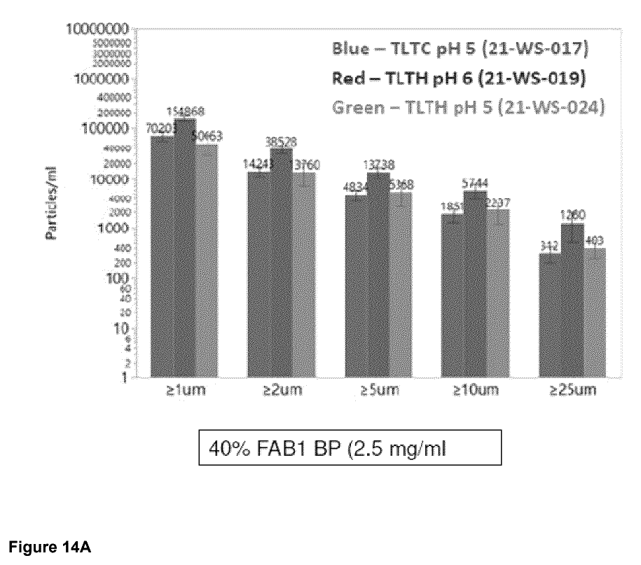

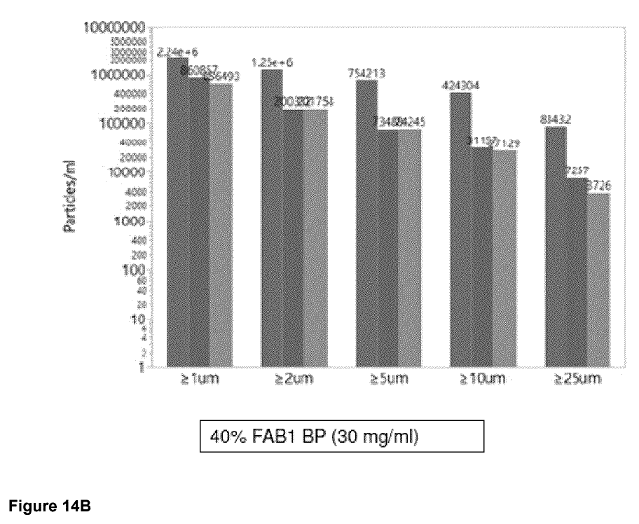

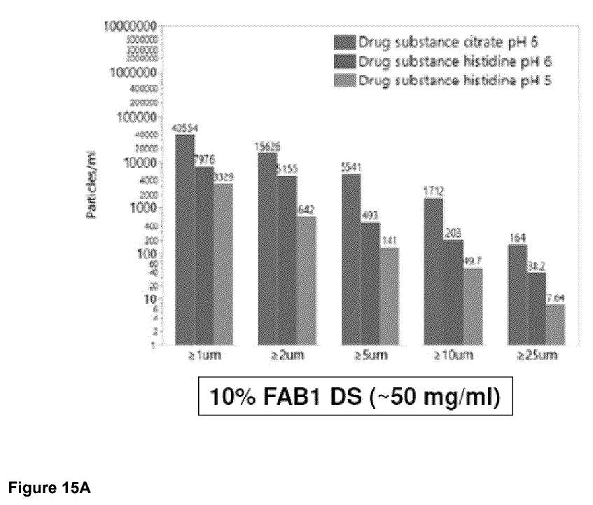

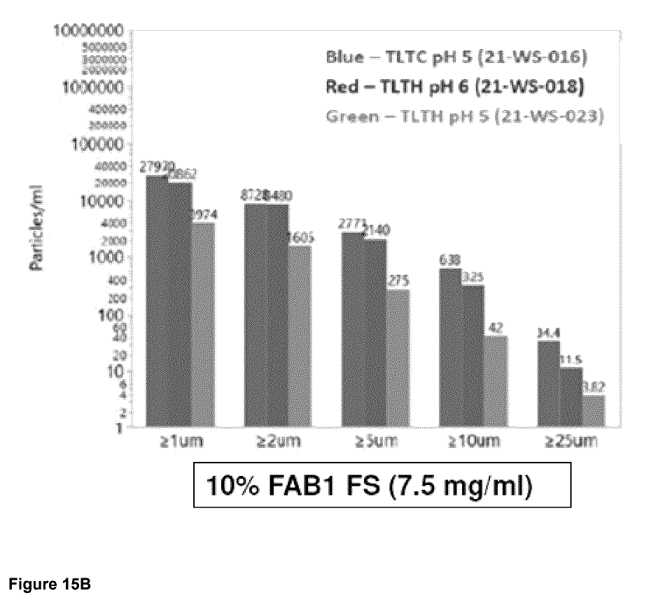

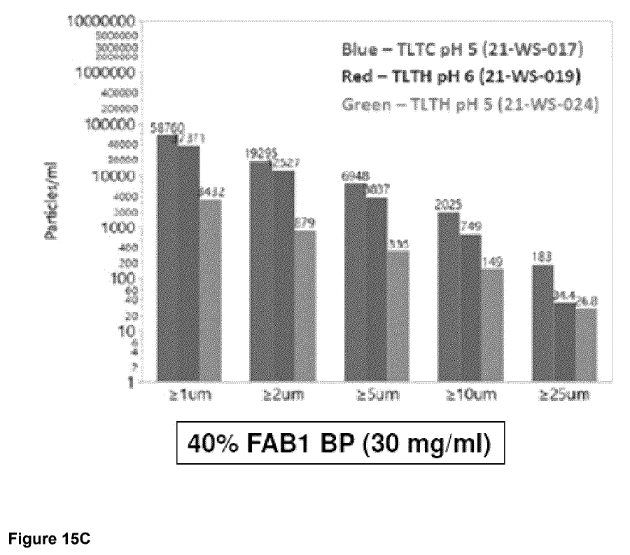

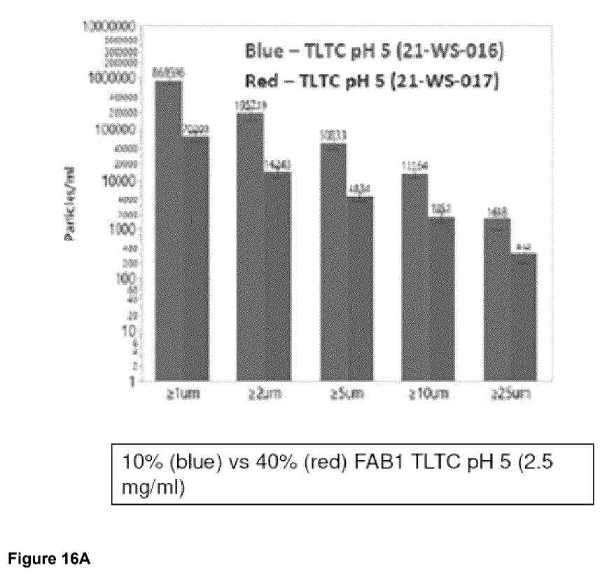

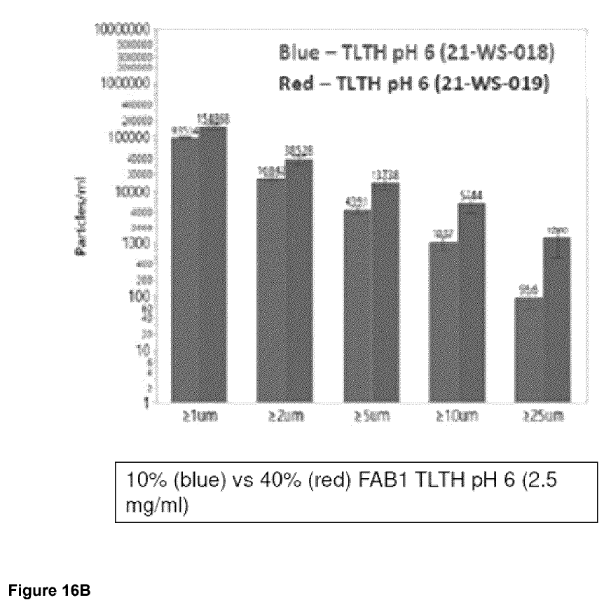

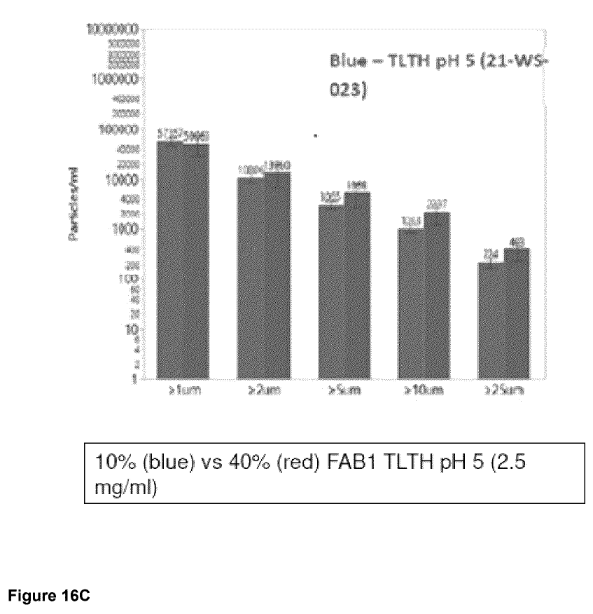

A critical readout for the dry powder formulation is the protein aggregation following reconstitution. Using Mass Flow Imaging (MFI), the number of particles were counted in powder reconstituted to two concentrations, 2.5 mg/ml protein and the feedstock concentration of 30 mg/ml. FIG. 13 shows the number of particles present in the tested powder formulations (TLTC pH 5 (21-WS-016); TLTH pH 6 (21-WS-018); and TLTH pH 5 (21-WS-023)). Particle counts were significantly higher with the TLTC formulation compared with TLTH, having “low” particle counts at 2.5 mg/ml and at feedstock concentration. FIG. 14 shows the number of particles in the tested powder formulations (TLTC pH 5 (21-WS-017); TLTH pH 6 (21-WS-019); and TLTH pH 5 (21-WS-024)). Particle counts were higher with the TLTC formulation compared to TLTH at feedstock concentration but not at 2.5 mg/ml. FIG. 15 shows the number of particles in the tested formulations (Drug substance citrate pH 5; Drug substance histidine pH 6; Drug substance histidine pH 5). Notably, DS in TLTC had higher particle counts than DS in TLTH where pH 5 was lower than pH 6. The same trend was observed for FS (FIGS. 15B and 15). FIG. 16 compares reconstituted powder with 10% and 40% FAB1 across the different formulations, and the lowest particle counts were observed for TLTH, pH 5 (see FIG. 16C).

Together, FIGS. 13 to 16 show that particle counts in DS, FS and reconstituted BP all followed the same pattern with lowest counts in TLTH, pH 5, slightly higher in TLTH, pH 6, and significantly higher in TLTC, pH 5. Due to the significantly higher levels of protein aggregation in the citrate-containing buffers, the histidine buffer was considered superior.

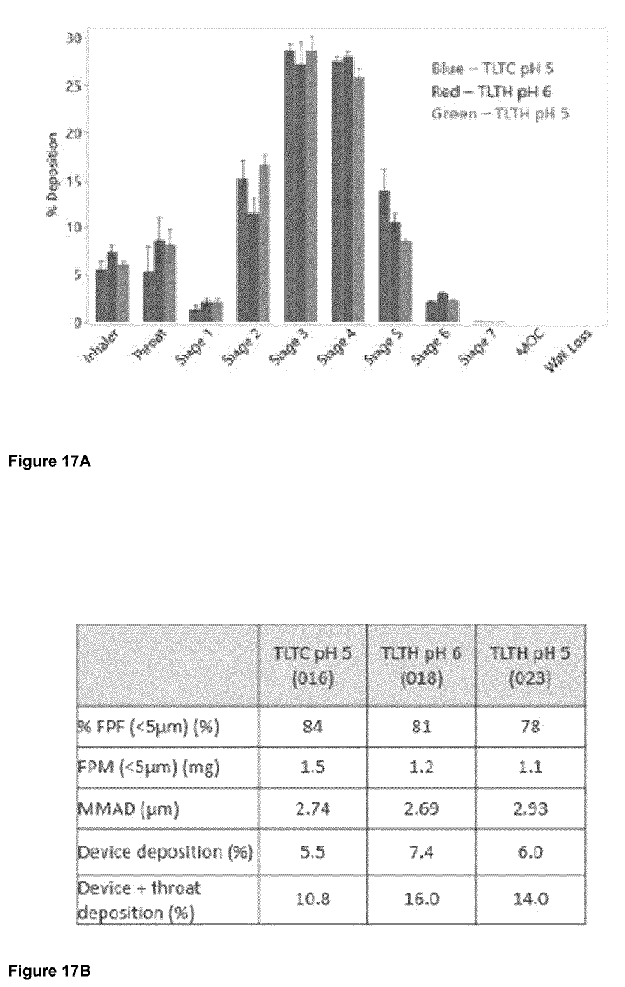

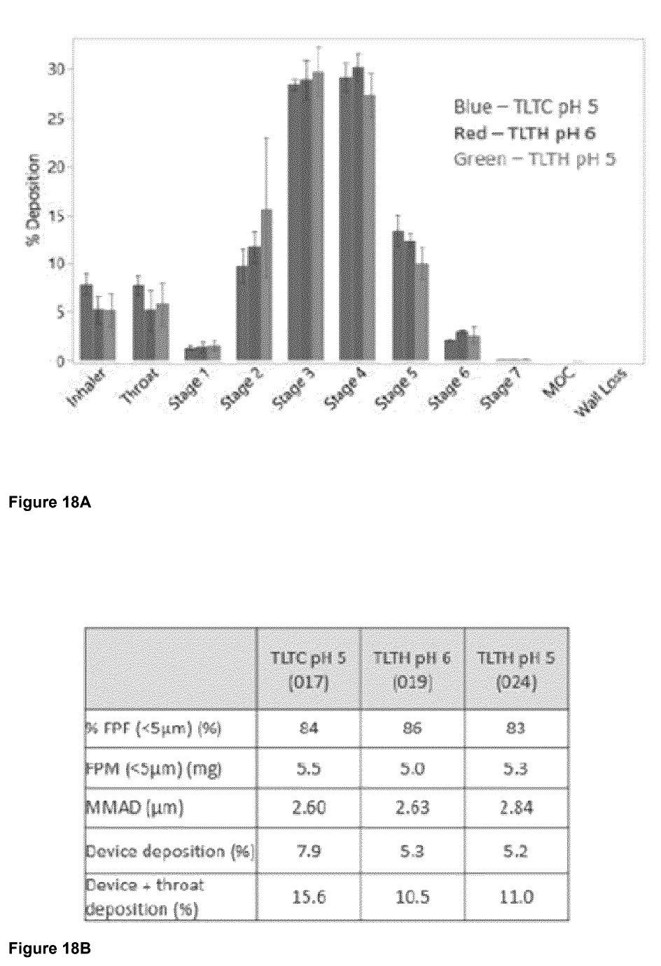

Finally, aerosol performance of the 10% and 40% lots was tested using a cascade impactor for pharmaceutical inhaler testing (see FIGS. 17 and 18). The deposition profiles for FAB1 were collected using a flow rate of 30.0±1.0 L/min and a Next Generation Impactor, as described by United States Pharmacopeia (USP)<601> Apparatus 6. The Next Generation Impactor consists of seven stages, or sample receptacles, that are rated at different mass median aerodynamic diameter values which depend on the flow rate, the NGI is also fitted with an USP induction port, “throat”, to accommodate the inhaler device. The inhaler device is “actuated” when a vacuum pump imposes a given flow rate, simulating a patient inspiration through the device's mouthpiece. Active agent amounts left in the device, including capsule, as well on throat, and on each of the stages is determined with an appropriate analytical method after each actuation. Aerosol performance is comparable across the formulations, with a very high (>80%) fine particle fraction (FPF) (<5 μm) and favourable median mass aerodynamic diameter (MMAD) of 2.5 microns (±0.5). The device deposition was low for all formulations, and lowest for the TLTH formulations.

The above results indicate that the histidine buffer is superior to the citrate buffer in terms of keeping the protein aggregation low.

### Example 2—Study 2: TLTH with PS80 and Trileucine/Leucine

### Experimental

Study 2 was designed to explore the formulation space for histidine and the shell-forming excipients leucine and trileucine. The first goal was to study the effect of reduced histidine on the particle surface by decreasing the total amount of histidine in the formulation (from 5% to 1.3%) or by increasing the leucine/trileucine ratio. Addition of a small amount of PS80 was also explored.

A design of experiments (DOE) was established to explore the design space for the formulation components focusing on subvisible particle counts and aerosol performance and device deposition as critical read out parameters. Factors for evaluation were 1) reduction in histidine, 2) increase shell-forming excipients to decrease the amount of histidine on the surface, 3) addition of optimized amount of PS80. Addition of PS80 results in increased device deposition, although it significantly reduces the protein aggregation and keeps the particle counts low (see WO2021/083908). Increasing the shell forming components trileucine and leucine was explored to improve the moisture robustness of the powder. Finally, a formulation with very high leucine (37.5%) and no trileucine was added to study moisture robustness. Nine batches were produced as shown in the following table:

Study 2 Batches

| Lot | | FAB1 | Batch | FS |
|---|---|---|---|---|
| number | Formulation | Strength | size (g) | Vol (ml) |
| 21-WS-039 | TLTH, pH 5.5, 1.3% His | 10% | 15 | 200 |
| 21-WS-040 | TLTH, pH 5.5, 1.3% His | 40% | | |
| 21-WS-041 | TLTH, pH 5.5, 1.3% His, | 10% | | |
| | 2x TriLeu/Leu | | | |
| 21-WS-042 | TLTH, pH 5.5, 5% His, | 10% | | |
| | 2x TriLeu/Leu | | | |
| 21-WS-043 | TLTH, pH 5.5, 1.3% His, | 10% | | |
| | 0.4% PS80 | | | |
| 21-WS-044 | TLTH, pH 5.5, 1.3% His, | 40% | | |
| | 0.4% PS80 | | | |
| 21-WS-045 | TLTH, pH 5.5, 5% His | 10% | | |
| 21-WS-046 | TLTH, pH 5.5, 5% His | 40% | | |
| 21-WS-060 | TLH, pH 5.5, 1.3% His, | 10% | | |
| | 37.5% Leu | | | |

The composition of each batch is summarised in the following table:

Study 2 formulation targets

| | Component (% w/w) | | | | | | | | |
|---|---|---|---|---|---|---|---|---|---|
| | | | | 10% | 10% | 40% | | | |
| | | | 10% | FAB1 | FAB1 | FAB1 | | | 10% |
| | 10% | 40% | FAB1 | 5% His 2x | 1.3% | 1.3% | | | FAB1 |
| | FAB1 | FAB1 | 1.3% His | 3Leu/Leu, | His | His | 10% | 40% | 1.3% His |
| | 1.3% | 1.3% | 2x Leu/ | 0.4% | 0.4% | 0.4% | FAB1 | FAB1 | 37.5% |
| | His | His | trileu | PS80 | PS80 | PS80 | 5% His | 5% His | Leu |
| Lot | 21- | 21- | 21-WS- | 21-WS- | 21- | 21- | 21- | 21- | 21-WS- |
| | WS- | WS- | 041 | 042 | WS- | WS- | WS- | WS- | 060 |
| | 039 | 040 | | | 043 | 044 | 045 | 046 | |
| FAB1 | 10.0 | 40.0 | 10.0 | 10.0 | 10.0 | 40.0 | 10.0 | 40.0 | 10.0 |
| Trehalose | 76.17 | 46.17 | 63.67 | 59.69 | 75.77 | 45.77 | 72.59 | 42.59 | 53 |
| Leucine | 10.50 | 10.50 | 21.0 | 21.0 | 10.5 | 10.5 | 10.5 | 10.5 | 37.5 |
| Trileucine | 2.0 | 2.0 | 4.0 | 4.0 | 2.0 | 2.0 | 2.0 | 2.0 | — |
| PS80 | — | — | — | 0.4 | 0.4 | 0.4 | — | — | — |
| L-Histidine | 0.25 | 0.25 | 0.25 | 0.93 | 0.25 | 0.25 | 0.93 | 0.93 | 0.23 |
| Histidine- | 1.08 | 1.08 | 1.08 | 3.98 | 1.08 | 1.08 | 3.98 | 3.98 | 1.07 |
| HCl | | | | | | | | | |

### Results

The following table is summary of the study processing results, including actual processing parameters and yields.

Study 2 processing results.

| | | Lot | | | | | | | | |
|---|---|---|---|---|---|---|---|---|---|---|
| | | 21-WS- | 21-WS- | 21-WS- | 21-WS- | 21-WS- | 21-WS- | 21-WS- | 21-WS- | 21-WS- |
| | | 039 | 040 | 041 | 042 | 043 | 044 | 045 | 046 | 060 |
| | | Description | | | | | | | | |
| | | | | | 10% | | | | | |
| | | | | | TLTH | | | | | |
| | | | | 10% | 5% His | 10% | 40% | | | 10% |
| | | | | TLTH | 2x Leu/ | TLTH | TLTH | | | TLTH |
| | | 10% | 40% | 1.3% His | trileu, | 1.3% His | 1.3% His | 10% | 40% | 1.3% His |
| | | TLTH | TLTH | 2x Leu/ | 0.4% | 0.4% | 0.4% | TLTH | TLTH | 37.5% |
| | | 1.3% His | 1.3% His | Trileu | PS80 | PS80 | PS80 | 5% His | 5% His | Leu |
| Form. | Batch size (g) | 15 | 15 | 15 | 15 | 15 | 15 | 15 | 15 | 15 |
| | Solids loading | 75 | 75 | 75 | 75 | 75 | 75 | 75 | 75 | 75 |
| | (mg/ml) | | | | | | | | | |
| | Feedstock (FS) | 200.00 | 200.00 | 200.00 | 200.00 | 200.00 | 200.00 | 200.00 | 200.00 | 200.00 |
| | Volume (ml) | | | | | | | | | |

| | | | | | | | | | |
|---|---|---|---|---|---|---|---|---|---|
| Yield (based on batch | 80.7 | 70.7 | 63.3 | 60.1 | 87.0 | 82.3 | 87.6 | 83.2 | 84.6 |
| size) (%) | | | | | | | | | |
| Yield (based on FS | 85.0 | 74.5 | 84.8 | 80.1 | 92.9 | 87.9 | 81.6 | 77.3 | 87.8 |
| mass) (%) | | | | | | | | | |
| FS density (g/ml) | 1.0238 | 1.0213 | 1.0214 | 1.0207 | | | | | |
| FS pH | 5.3 | 5.5 | 5.5 | 5.5 | 5.4 | 5.4 | 5.4 | 5.3 | 5.4 |

1) Yield based on batch size is calculated as the net collector yield divided by the nominal batch size.
2) Yield based on FS mass is calculated as the net collector yield divided by the actual FS mass.

Study 2 solid state testing.

| | | 10% FAB1 | | | | | | | | |
|---|---|---|---|---|---|---|---|---|---|---|
| | | | TLTH | | | | | 40% FAB1 | | |
| | | | 1.3% | TLTH 5% | TLTH | | TLTH | | TLTH | |
| | | TLTH | His 2x | His 2x | 1.3% His | TLTH | 1.3% His | TLTH | 1.3% His | TLTH |
| | | 1.3% | Leu/trileu | 3Leu/Leu, | 0.4% PS80 | 5% His | 37.5% Leu | 1.3% His | 0.4% PS80 | 5% His |
| | | His (21- | (21-WS- | 0.4% PS80 | (21-WS- | (21-WS- | (21-WS- | (21-ws- | (21-WS- | (21-WS- |
| | | WS-039) | 041) | (21-WS-42) | 043) | 045) | 60) | 40) | 044) | 046) |
| Drug | mg/ml | 54.0 | 53.0 | 53.0 | 53.9 | 52.8 | 59.1 | 54.0 | 53.0 | 52.8 |
| Substance | % recovery | 98 | 96 | 96 | 98 | 96 | 98 | 98 | 96 | 96 |
| (DS) protein | | | | | | | | | | |
| FS protein | mg/ml | 7.4 | 7.5 | 7.6 | 7.6 | 7.6 | 4.9 | 30.0 | 29.8 | 30.1 |
| | % recovery | 99 | 100 | 102 | 101 | 101 | 98.8 | 100 | 99 | 100 |
| Bulk powder | % w/w | 9.9 | 10.2 | 10.2 | 9.8 | 10.0 | 9.7 | 38.8 | 39.8 | 39.3 |
| (BP) protein | % recovery | 99.5 | 101.6 | 101.6 | 98.3 | 99.6 | 97.4 | 97 | 99.4 | 98.3 |
| DS High | % MPP | 100 | 100 | 100 | 100 | 100 | 100 | 100 | 100 | 100 |
| performance | % | 0 | 0 | 0 | 0 | 0 | 0 | 0 | 0 | 0 |
| size exclusion | aggregates | | | | | | | | | |
| chromatography | | | | | | | | | | |
| (HP-SEC) | | | | | | | | | | |
| FS HP-SEC | % MPP | 100 | 100 | 100 | 100 | 100 | 99.4 | 100 | 100 | 100 |
| | % | 0 | 0 | 0 | 0 | 0 | 0.6 | 0 | 0 | 0 |
| | aggregates | | | | | | | | | |
| BP HP-SEC | % MPP | 100.0 | 99.9 | 99.9 | 100.0 | 100.0 | 99.6 | 99.9 | 99.9 | 99.9 |
| | % | 0.0 | 0.1 | 0.1 | 0.0 | 0.0 | 0.4 | 0.1 | 0.1 | 0.1 |
| | aggregates | | | | | | | | | |
| Primary | d10 (μm) | 0.5 | 0.5 | 0.5 | 0.5 | 0.5 | 0.5/0.4¹ | 0.5 | 0.5 | 0.5 |
| particle size | d50 (μm) | 1.5 | 1.6 | 1.7 | 1.5 | 1.6 | 1.7/1.3¹ | 1.7 | 1.7 | 1.7 |
| distribution | d90 (μm) | 3.5 | 3.6 | 3.6 | 3.5 | 3.6 | 4.5/3.1¹ | 3.9 | 3.9 | 3.9 |
| (pPSD) | Span | 2.0 | 1.9 | 1.9 | 2.0 | 1.9 | 2.4/2.0¹ | 2.0 | 1.9 | 2.0 |

| | | | | | | | | | |
|---|---|---|---|---|---|---|---|---|---|
| Moisture content (%) | 0.9 | 0.9 | 0.7 | 0.7 | 0.6 | 0.6 | 1.0 | 0.9 | 1.1 |
| Glass transition temperature | 100 | 100 | 98 | 102 | 100 | 108 | 104 | 81, | 100 |
| (Tg) (° C.) | | | | | | | | 104² | |
| Compressed Bulk Density | — | — | — | — | — | 0.6969 | — | — | — |
| (cBD) (g/cm³) | | | | | | | | | |
| Specific Surface Area (SSA) | — | — | — | — | — | 5.4687 | — | — | — |
| (m²/g) | | | | | | | | | |

¹Larger particle size than expected on initial testing, so pPSD test repeated.
²Values refer to Tg onset and 2^{nd} Tg onset.

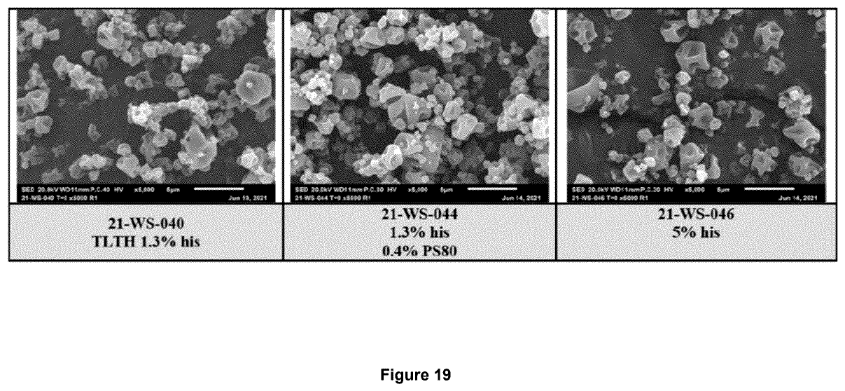

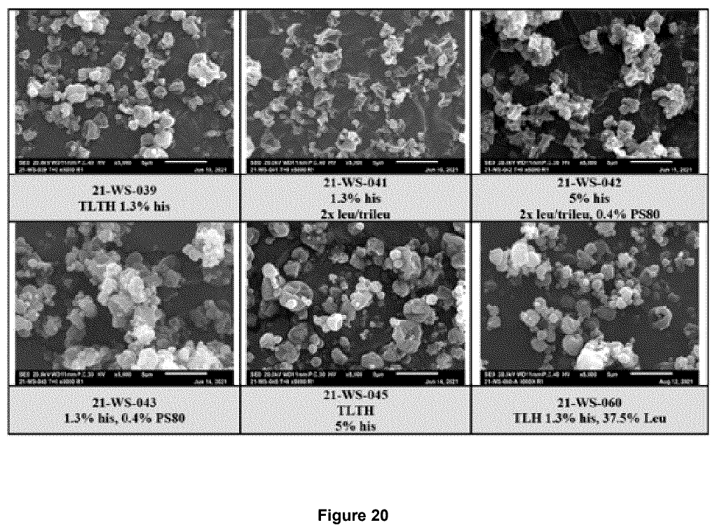

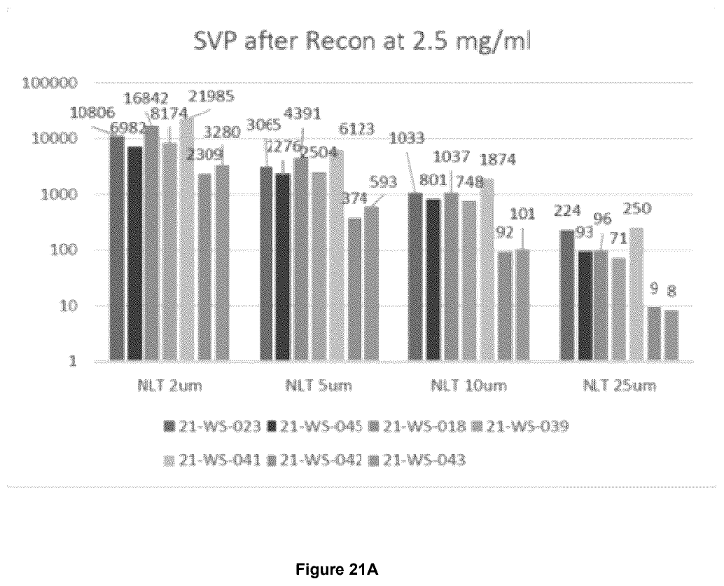

Scanning Electron Microscopy (SEM) analysis of the formulations demonstrated good morphology with similar appearance for both the TLTC and TLTH formulations (FIGS. 19 and 20).

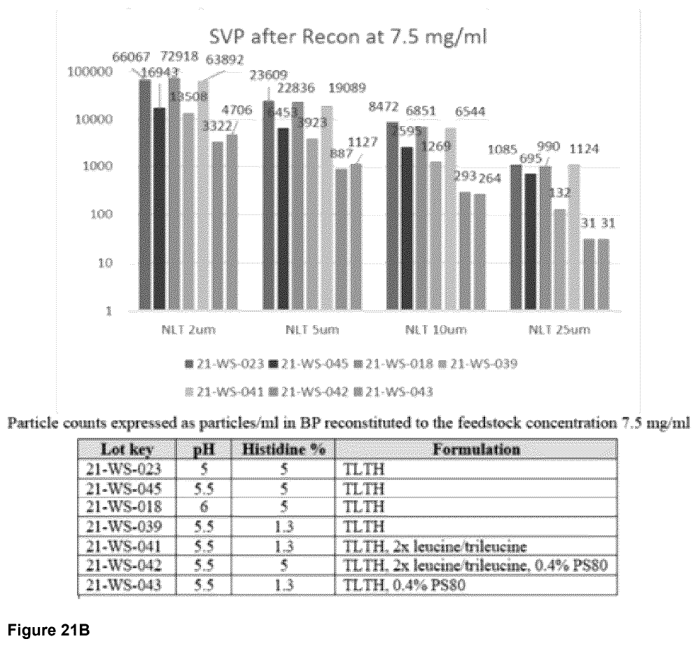

MFI results for the 10% FAB1 are shown in FIG. 21. All formulations showed low particle counts compared with the TLTC formulation (21-WS-016-see FIG. 16). Addition of PS80 further reduced the particle counts (21-WS-042 and 043). Doubling leucine and trileucine had no impact on the particle counts.

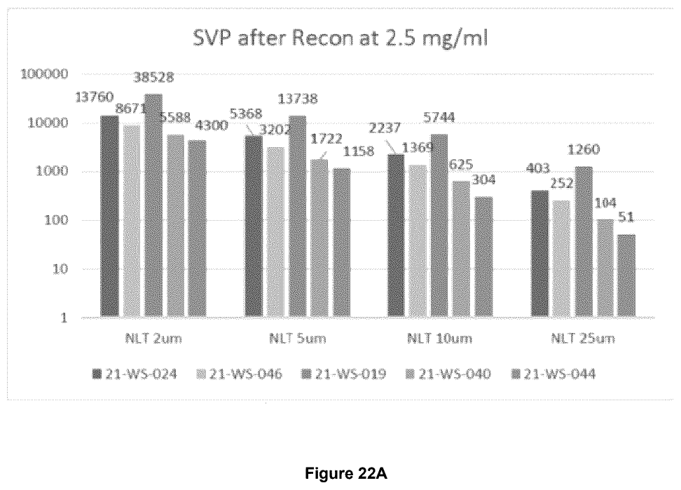

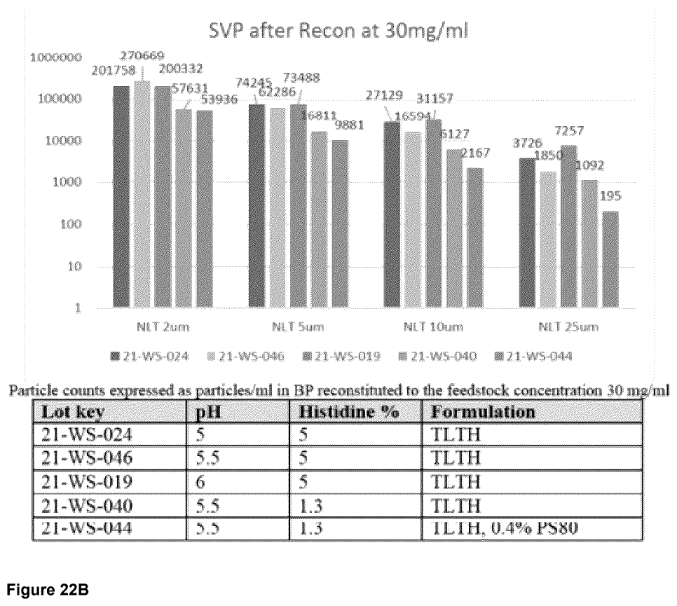

MFI results for the 40% FAB1 are shown in FIG. 22. All formulations showed low particle counts compared with the TLTC formulation (21-WS-017). Addition of PS80 further reduced the particle counts (21-WS-044). There is a trend towards lower particle counts with 1.3% histidine compared with 5% histidine. Similar results for pH 5, pH 5.5 and pH 6.

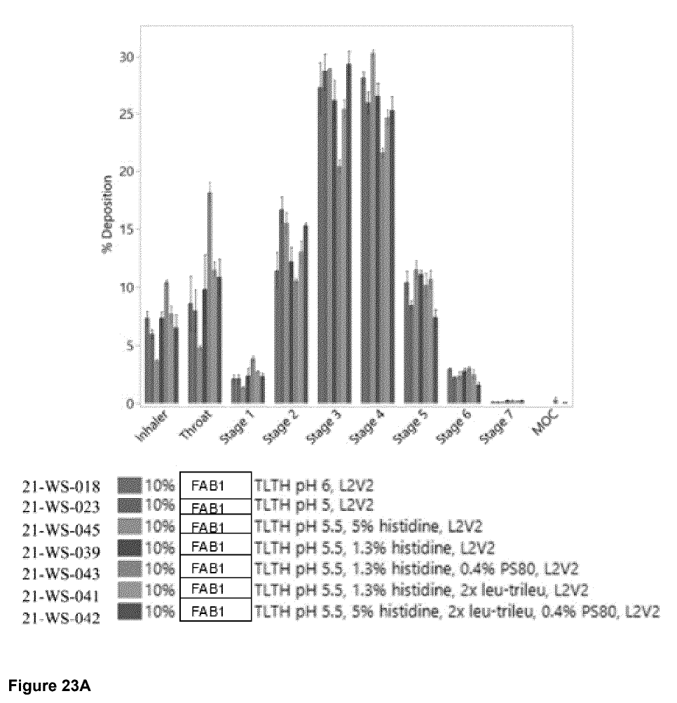

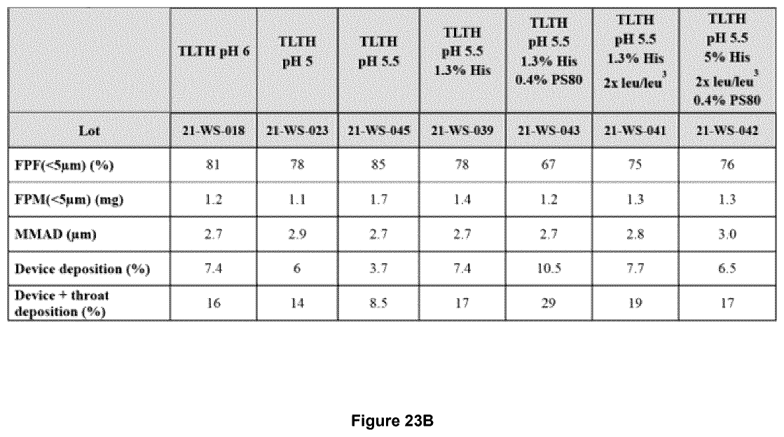

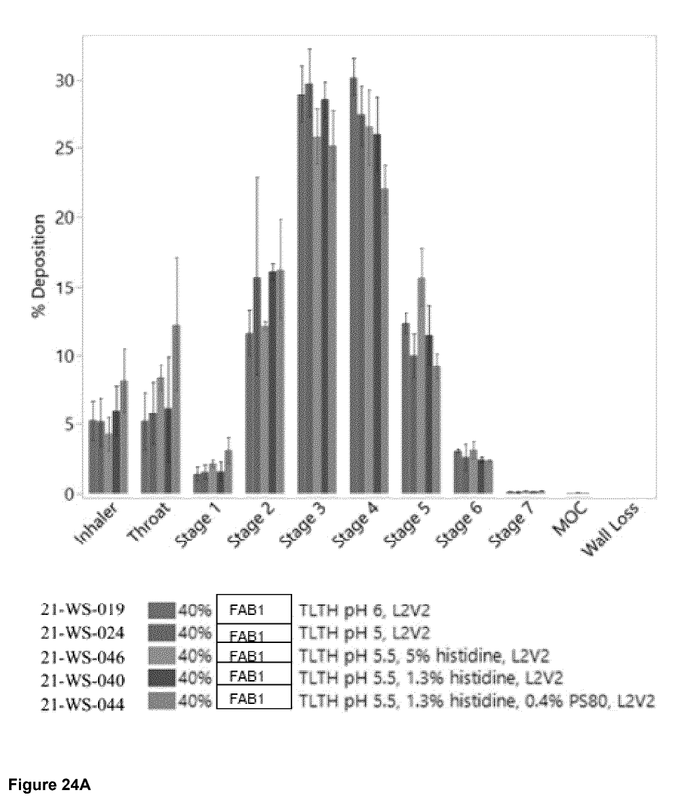

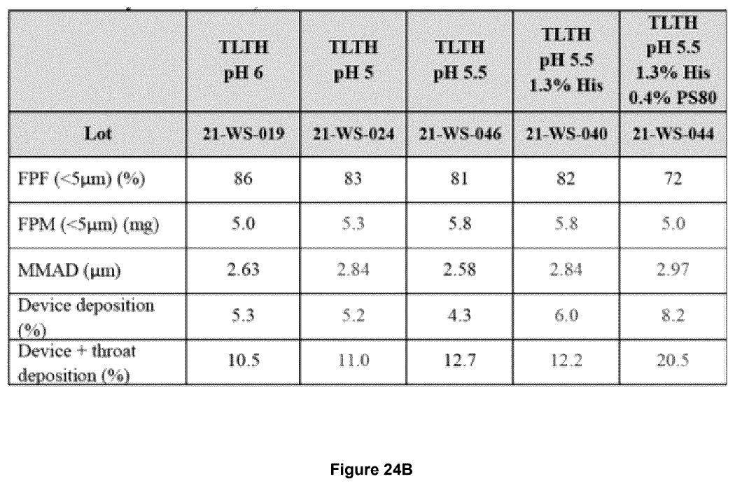

Finally, aerosol performance of the 10% and 40% lots was tested using a cascade impactor for pharmaceutical inhaler testing (see FIGS. 23 and 24).

When comparing TLTH formulations with 5% histidine and pH at 5, 5.5 or 6, no significant differences were observed for solid state properties (Table “Study 2 solid state testing”), aerosol performance and protein aggregation (FIGS. 23 and 24). In the DOE analysis and modelling of histidine vs PS80 it was concluded that higher histidine and lower PS80 levels were beneficial to lower device deposition, while only PS80 affected the protein aggregations. Addition of high leucine/trileucine also had no significant impact on the device deposition or protein aggregation. Moisture content and glass transition temperature were similar for all formulations. PS80 at a low level (0.4%) showed tendency of stickiness with lower spray drying yield and higher device deposition (FIGS. 23 and 24) but kept, as expected, the protein aggregations to a minimal (Table “Study 2 solid state testing”).

### Conclusion

Together these data show that higher concentrations of histidine combined with lower levels of PS80 reduces device deposition. The formulation containing TLTH at pH 5.5 keeping trileucine at 2.0% w/w and leucine at 10.5% w/w with no addition of PS80 showed favourable properties. Therefore, the inventors further optimised this formulation by investigating the optimal histidine concentration.

### Example 3—Study 3: TLTH Optimal Histidine Levels

### Experimental

Study 3 investigates the optimum histidine concentration for the TLTH formulation. Three levels of histidine were explored to select the optimal concentration. Six batches were produced as shown in the following table:

Study 3 Batches

| | | FAB1 | Batch | FS |
|---|---|---|---|---|
| Lot number | Formulation | Strength | size (g) | Vol (ml) |
| 21-WS-055 | TLTH, pH 5.5, 1.3% His | 10% | 15 | 200 |
| 21-WS-056 | TLTH, pH 5.5, 3.14% His | 10% | | |
| 21-WS-057 | TLTH, pH 5.5, 5% His | 10% | | |
| 21-WS-058 | TLTH, pH 5.5, 3.14% His | 40% | | |
| 21-WS-059 | TLTH, pH 5.5, 5% His | 40% | | |
| 21-WS-061 | TLTH, pH 5.5, 1.3% His | 40% | | |
| 21-WS-045 | TLTH, pH 5.5, 5% His | 10% | | |
| 21-WS-046 | TLTH, pH 5.5, 5% His | 40% | | |
| 21-WS-060 | TLH, pH 5.5, 1.3% His, | 10% | | |
| | 37.5% Leu | | | |

The composition of each batch is summarized in the following table:

Study 3 formulation targets

| | Component (% w/w) | | | | | |
|---|---|---|---|---|---|---|
| | 10% FAB1 | 10% FAB1 | 10% FAB1 | 40% FAB1 | 40% FAB1 | 40% FAB1 |
| | 1.3% His | 3.14% His | 5% His | 3.14% His | 5% His | 1.3% His |
| Lot | 21-WS-055 | 21-WS-056 | 21-WS-057 | 21-WS-058 | 21-WS-059 | 21-WS-061 |
| FAB1 | 10.0 | 10.0 | 10.0 | 40.0 | 40.0 | 40.0 |
| Trehalose | 76.20 | 74.36 | 72.52 | 44.36 | 42.52 | 46.16 |
| Leucine | 10.50 | 10.50 | 10.50 | 10.50 | 10.50 | 10.50 |
| Trileucine | 2.00 | 2.00 | 2.00 | 2.00 | 2.00 | 2.00 |
| L-Histidine | 0.23 | 0.55 | 0.88 | 0.55 | 0.88 | 0.37 |
| Histidine-HCl | 1.07 | 2.59 | 4.10 | 2.59 | 4.10 | 0.97 |

### Results

The following table shows a summary of the study 3 processing results, including actual processing parameters and yields:

Study processing results

| | | Lot | | | | | |
|---|---|---|---|---|---|---|---|
| | | 21-WS-055 | 21-WS-056 | 21-WS-057 | 21-WS-058 | 21-WS-059 | 21-WS-061 |
| | | Description | | | | | |
| | | 10% TLTH, | 10% TLTH, | 10% TLTH, | 40% TLTH, | 40% TLTH, | 40% TLTH, |
| | | 1.3% His | 3.14% His | 5% His | 3.14% His | 5% His | 1.3% His |
| Formulation | Batch | 15 | 15 | 15 | 15 | 15 | 15 |
| | size (g) | | | | | | |
| | Solids | 75 | 75 | 75 | 75 | 75 | 75 |
| | loading | | | | | | |
| | (mg/ml) | | | | | | |
| | Feedstock | 200.00 | 200.00 | 200.00 | 200.00 | 200.00 | 200.00 |
| | (FS) | | | | | | |
| | Volume | | | | | | |
| | (ml) | | | | | | |

| | | | | | | |
|---|---|---|---|---|---|---|
| Yield (based on batch | 79.9 | 85.2 | 81.8 | 74.3 | 66.7 | 85.2 |
| size) (%) | | | | | | |
| Yield (based on FS | 83.4 | 88.9 | 85.4 | 77.4 | 69.6 | 89.2 |
| mass) (%) | | | | | | |
| FS pH | 5.4 | 5.4 | 5.5 | 5.4 | 5.4 | 5.7 |

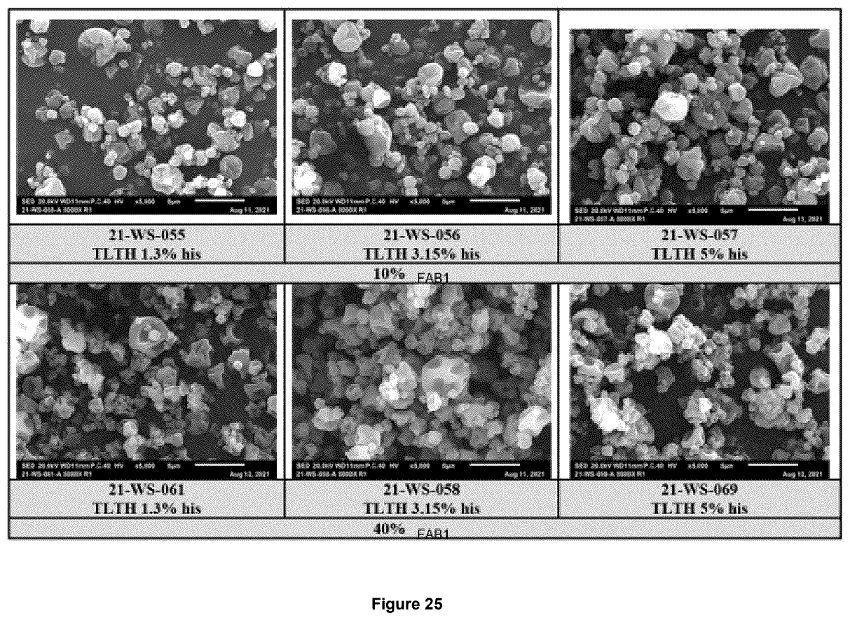

Solid state testing results are shown in the following table and SEM images are shown in FIG. 25.

Study 3 solid state testing

| | | 10% FAB1 | | | 40% FAB1 | | |
|---|---|---|---|---|---|---|---|
| | | TLTH | TLTH | | TLTH | TLTH | |
| | | 1.3% His | 3.14% | TLTH 5% | 1.3% His | 3.14% | TLTH 5% |
| | | (21-WS- | His (21- | His (21- | (21-WS- | His (21- | His (21- |
| | | 055) | WS-056) | WS-057) | 061) | WS-058) | WS-059) |
| Drug | mg/ml | 59.1 | 59.1 | 59.1 | 52.4 | 59.1 | 59.1 |
| Substance (DS) | % recovery | 98 | 98 | 98 | 96 | 98 | 98 |
| protein | | | | | | | |
| FS protein | mg/ml | 7.4 | 7.4 | 7.4 | 28.6 | 29.5 | 29.4 |
| | % recovery | 99.1 | 98.4 | 98.9 | 95.5 | 98.1 | 98.1 |
| Bulk powder | % w/w | 10.2 | 10.3 | 10.3 | 37.9 | 41.0 | 40.8 |
| (BP) protein | % recovery | 102.3 | 103.4 | 102.6 | 94.8 | 102.6 | 102.1 |
| DS High | % MPP | 100 | 100 | 100 | 100 | 100 | 100 |
| performance | % aggregates | 0 | 0 | 0 | 0 | 0 | 0 |
| size exclusion | | | | | | | |
| chromatography | | | | | | | |
| (HP-SEC) | | | | | | | |
| FS HP-SEC | % MPP | 100 | 100 | 100 | 100 | 100 | 100 |
| | % aggregates | 0 | 0 | 0 | 0 | 0 | 0 |
| BP HP-SEC | % MPP | 100.0 | 100.0 | 100.0 | 99.9 | 99.9 | 99.9 |
| | % aggregates | 0.0 | 0.0 | 0.0 | 0.1 | 0.1 | 0.1 |
| Primary | d10 (μm) | 0.5 | 0.5 | 0.5 | 0.6/0.4 | 0.5 | 0.5 |
| particle size | d50 (μm) | 1.6 | 1.6 | 1.6 | 2.1/1.7 | 1.7 | 1.6 |
| distribution | d90 (μm) | 3.6 | 3.6 | 3.7 | 5.0/3.9 | 4.1 | 3.9 |
| (pPSD) | Span | 2.0 | 2.0 | 2.0 | 2.2/2.0 | 2.1 | 2.1 |

| | | | | | | |
|---|---|---|---|---|---|---|
| Moisture content (%) | 0.7 | 0.7 | 0.7 | 0.9 | 1.0 | 1.0 |
| Glass transition temperature | 101, 103 | 100, 102 | 100, 101 | 116 | 99, 105 | 100, 102 |
| (Tg) (° C.) | | | | | | |
| Compressed Bulk Density (cBD) | 0.6131 | 0.6901 | 0.6558 | 0.5529 | 0.6226 | 0.5378 |
| (g/cm³) | | | | | | |
| Specific Surface Area (SSA) (m²/g) | 4.8447 | 5.2046 | 4.9938 | 5.7128 | 5.7473 | 5.8758 |

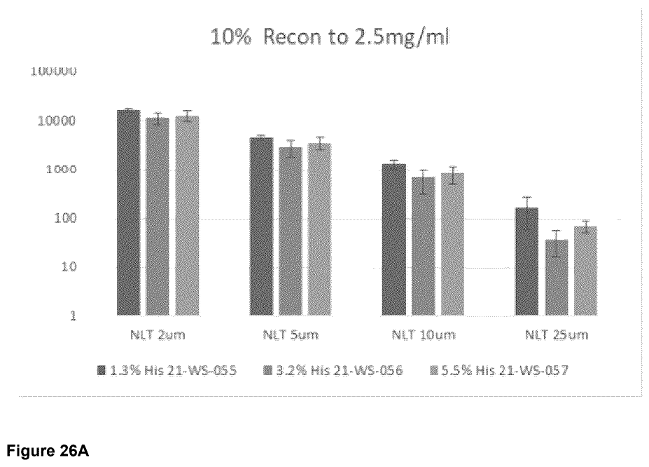

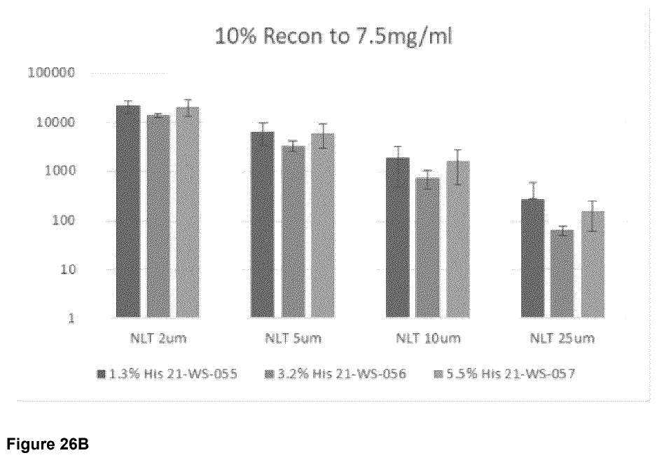

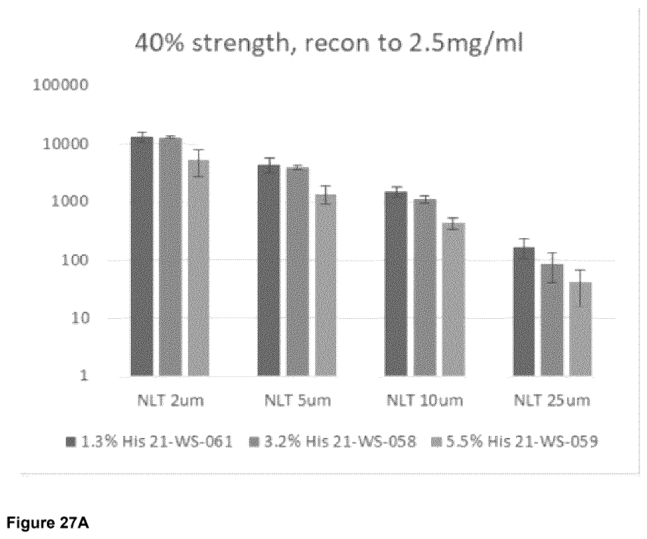

All TLTH formulations show excellent solid-state properties based on the following criteria: D90<5 μm (90% of the particles less than 5 μm), % water <5%, Tg>80° C. at 2% water content. Subvisible particles detected by MFI were low for all TLTH formulations (see FIGS. 26 and 27).

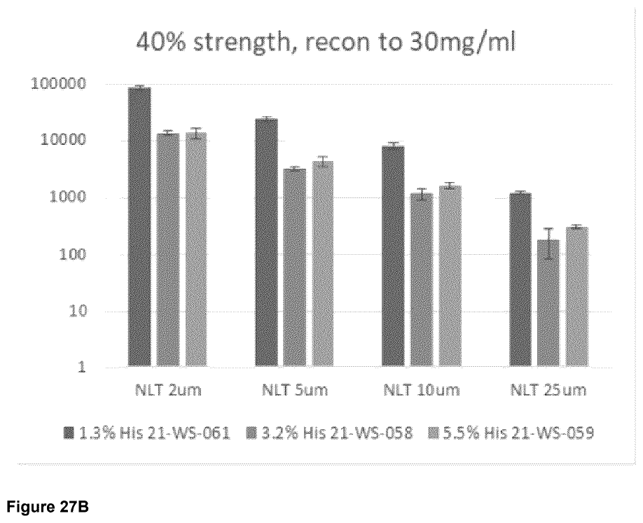

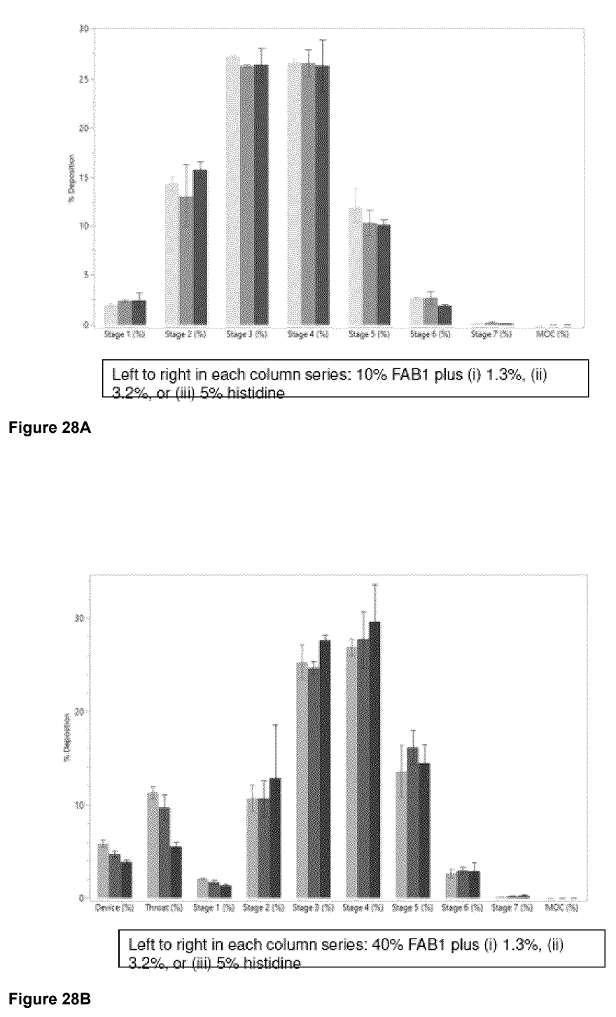

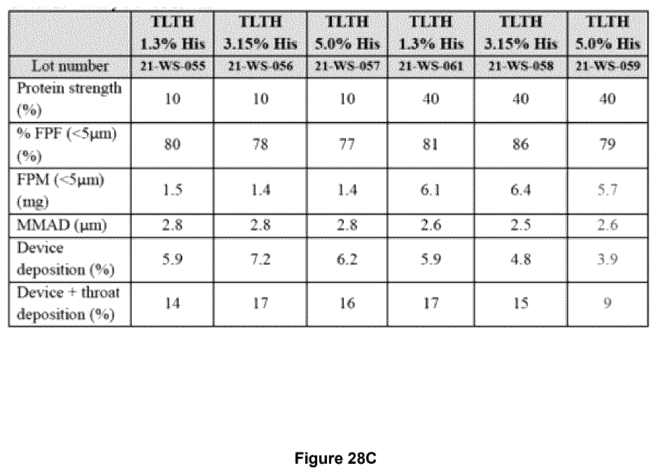

The NGI results for the 10% and 40% are plotted in FIGS. 28A and B and summarised in FIG. 28C, and these data indicate that aerosol performance is very good at all histidine concentrations.

### Conclusions

Three levels of histidine 1.3%, 3.14% and 5% were explored at 10% and 40% protein strength to establish optimal histidine concentration. There was no significant difference in solid-state or aerosol performance between the different formulations and protein aggregations were low with a small trend for lower numbers at the higher histidine levels.

### Example 4—Study 4:1 Month Stability of TLTH

### Experimental

Study 4 was added to gain insight in the stability of the TLTH formulation with optimized histidine and pH. Powder and filled capsules previously manufactured in studies 1-3 were used for the stability testing, as shown in the following table. High (5%) and low (1.3%) histidine formulations were set down at 40° C./75% RH (protected with foil overwrap and desiccant) to support TLTH as the proposed phase 2 formulation. NLT 2 g of powder for each lot was transferred into aluminum Tournaire containers and foil overwrapped with desiccant and a stability test was performed for 1 month at 40° C./75% RH. Capsules were filled and packed in foil pouches with desiccant and stored protected at 40° C./75% RH.

Study 4 Stability batches

| Lot number | Formulation | FAB1 Strength |
|---|---|---|
| 21-WS-039 | TLTH, pH 5.5, 1.3% His | 10% |
| 21-WS-040 | TLTH, pH 5.5, 1.3% His | 40% |
| 21-WS-045 | TLTH, pH 5.5, 5% His | 10% |
| 21-WS-046 | TLTH, pH 5.5, 5% His | 40% |
| 21-WS-056 | TLTH, pH 5.5, 3.14% His | 10% |
| 21-WS-058 | TLTH, pH 5.5, 3.14% His | 40% |

### Results

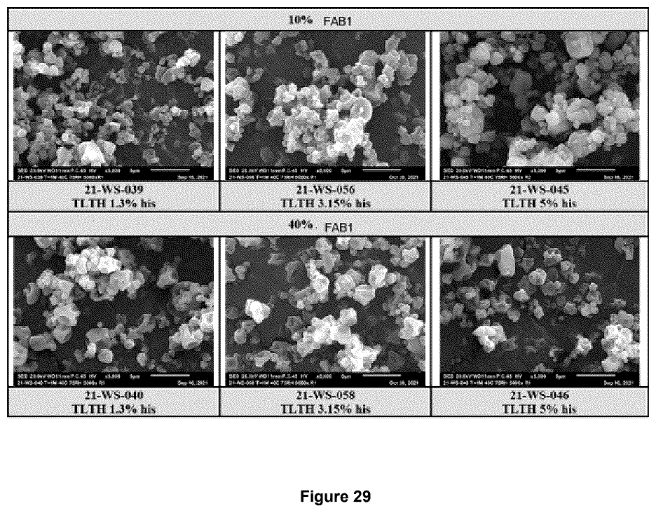

The following two tables summarise the solid-state stability results, and SEM images are shown in FIG. 29.

Stability results 10% FAB1 TLTH formulation.

| | | Formulation | | | | | |
|---|---|---|---|---|---|---|---|
| | | TLTH | | TLTH | | TLTH | |
| | | 1.3% His | | 3.14% His | | 5% His | |
| | | Lot number | | | | | |
| | | 21-WS-039 | | 21-WS-056 | | 21-WS-045 | |
| | | Time point (months) | | | | | |
| | | T = 0 | T = 1 | T = 0 | T = 1 | T = 0 | T = 1 |
| BP protein | % w/w | 9.9 | 9.43 | 10.3 | 9.81 | 10 | 9.59 |
| | % | 99.5 | 94.3 | 103.6 | 98.1 | 99.6 | 95.9 |
| | Recovery | | | | | | |
| BP | % MPP | 100 | 100 | 100 | 100 | 100 | 99.9 |
| HP-SEC | % | 0.0 | 0.0 | 0.0 | 0.0 | 0.0 | 0.1 |
| | Aggregates | | | | | | |
| pPSD | d10 (μm) | 0.5 | 0.4 | 0.5 | 0.5 | 0.5 | 0.4 |
| | d50 (μm) | 1.5 | 1.5 | 1.6 | 1.6 | 1.6 | 1.6 |
| | d90 (μm) | 3.5 | 3.5 | 3.6 | 3.6 | 3.6 | 3.6 |
| | Span | 2.0 | 2.1 | 2.0 | 2.0 | 1.9 | 2.0 |

| | | | | | | |
|---|---|---|---|---|---|---|
| Moisture content (%) | 0.9 | 1.2, | 0.7 | 0.7 | 0.6 | 1, |
| | | 1.6* | | | | 1.3* |

*KF was rerun to confirm a slight increase in moisture content in two of the formulations.

Stability results 40% FAB1 TLTH formulation.

| | | Formulation | | | | | |
|---|---|---|---|---|---|---|---|
| | | TLTH | | TLTH | | TLTH | |
| | | 1.3% His | | 3.14% His | | 5% His | |
| | | Lot number | | | | | |
| | | 21-WS-040 | | 21-WS-058 | | 21-WS-046 | |
| | | Time point (months) | | | | | |
| | | T = 0 | T = 1 | T = 0 | T = 1 | T = 0 | T = 1 |
| BP protein | % w/w | 38.3 | 36.4 | 41.0 | 39.3 | 39.3 | 37.8 |
| | % Recovery | 97.0 | 88.1, | 103 | 98.4 | 98.3 | 90.1, |
| | | | 91.0* | | | | 94.4* |
| BP HP-SEC | % MPP | 99.9 | 99.9 | 99.9 | 99.9 | 99.9 | 99.9 |
| | % Aggregates | 0.1 | 0.1 | 0.1 | 0.1 | 0.1 | 0.1 |
| pPSD | d10 (μm) | 0.5 | 0.4 | 0.5 | 0.5. | 0.5 | 0.4 |
| | d50 (μm) | 1.7 | 1.7 | 1.7 | 1.6 | 1.7 | 1.7 |
| | d90 (μm) | 3.9 | 3.9 | 4.1 | 3.7 | 3.9 | 3.9 |
| | Span | 2.0 | 2.1 | 2.1 | 2.0 | 2.0 | 2.1 |

| | | | | | | |
|---|---|---|---|---|---|---|
| Moisture content (%) | 1.0 | 1.1, | 1.0 | 1.0 | 1.1 | 1.2, |
| | | 1.2* | | | | 1.3* |

*KF was rerun to confirm a slight increase in moisture content in two of the formulations.

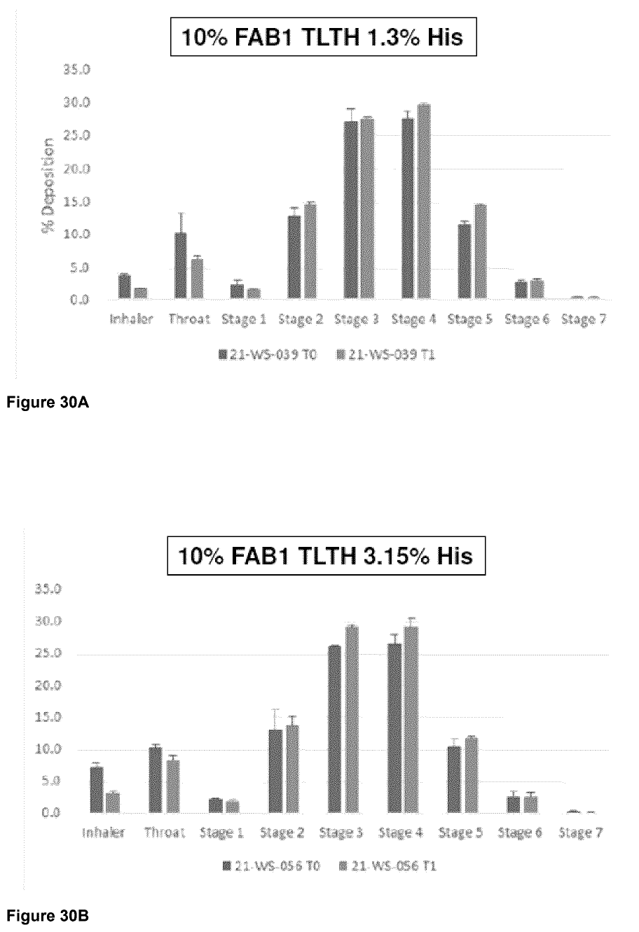

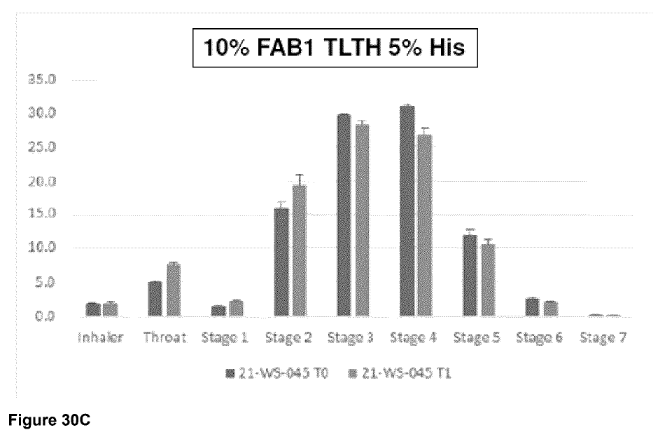

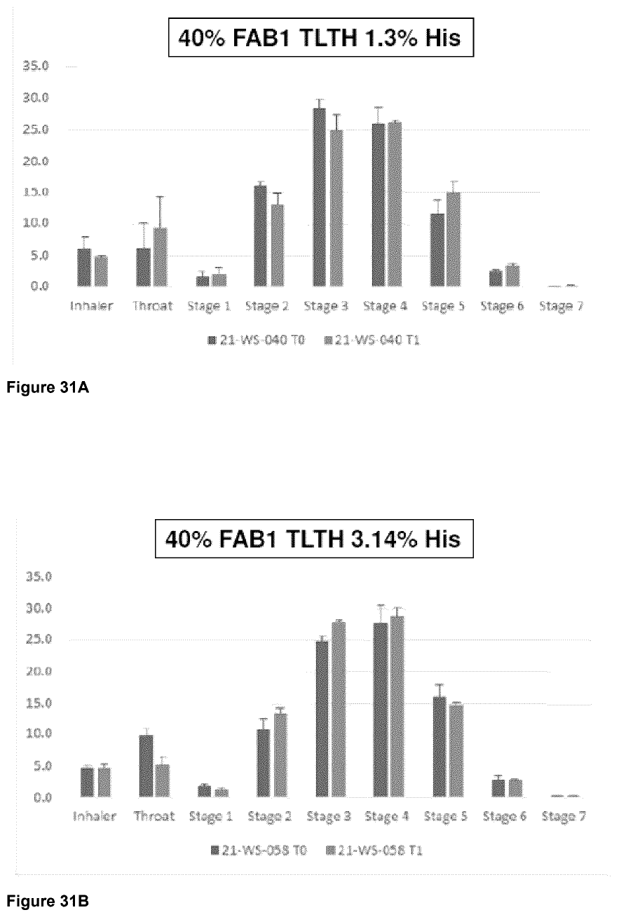

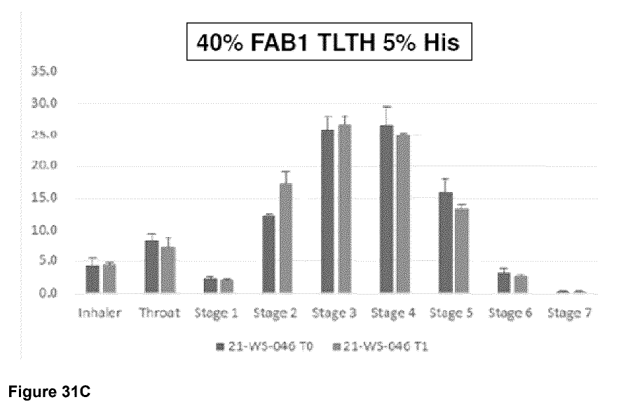

The NGI results for Study 4 are shown in FIGS. 30 and 31 and summarised in the following two tables:

NGI 1 Month Accelerated Stability Results 10% FAB1.

| | Formulation | | | | | |
|---|---|---|---|---|---|---|
| | TLTH | | TLTH | | TLTH | |
| | 1.3% His | | 3.14% His | | 5% His | |
| | Lot number | | | | | |
| | 21-WS-039 | | 21-WS-056 | | 21-WS-045 | |
| | Time point (months) | | | | | |
| | T = 0 | T = 1 | T = 0 | T = 1 | T = 0 | T = 1 |
| FPF (<5 μm) (%) | 78 | 83 | 78 | 81 | 84 | 78 |
| FPM (<5 μm) (mg) | 1.4 | 1.6 | 1.4 | 1.5 | 1.6 | 1.5 |
| MMAD (μm) | 2.7 | 2.6 | 2.7 | 2.7 | 2.7 | 2.9 |

NGI 1 Month Accelerated Stability Results 40% FAB1.

| | Formulation | | | | | |
|---|---|---|---|---|---|---|
| | TLTH | | TLTH | | TLTH | |
| | 1.3% His | | 3.14% His | | 5% His | |
| | Lot number | | | | | |
| | 21-WS-040 | | 21-WS-058 | | 21-WS-046 | |
| | Time point (months) | | | | | |
| | T = 0 | T = 1 | T = 0 | T = 1 | T = 0 | T = 1 |
| FPF (<5 μm) (%) | 82 | 80 | 81 | 85 | 81 | 80 |
| FPM (<5 μm) (mg) | 5.8 | 5.6 | 6.1 | 6.3 | 5.8 | 5.5 |
| MMAD (μm) | 2.8 | 2.6 | 2.5 | 2.6 | 2.6 | 2.8 |

| | | | Total | FAB1 | Deposited | | |
|---|---|---|---|---|---|---|---|
| | | Exposure | Aerosol | Aerosol | Dose | Animals | |
| | | Duration | Dose Level | Dose Level | Level | Main | |
| Group | Dose | (Minutes) | (mg/L) | (mg/L) | (mg/kg/d) | Study | Recovery |
| 1 | Air | 60 | LLOQ | LLOQ | 0 | 3M + 3F | 2M + 2F |
| | Control | | | | | | |
| 2 | Placebo | 60 | 2.5 | LLOQ | 0 | 3M + 3F | |
| | Control | | | | | | |
| 3 | Low Dose | 8 | 2.5 | 963 | 1.0 | 3M + 3F | |
| 4 | Mid Dose | 20 | 2.5 | 958 | 2.3 | 3M + 3F | 2M + 2F |
| 5 | High Dose | 60 | 2.5 | 893 | 7.1 | 3M + 3F | 2M + 2F |

### Conclusions

All formulations performed very well, and the formulation containing TLTH, pH 5.5 with 3.14% histidine (w/w) showed especially good stability.

### Example 5—In Vivo Toxicity Study

To test the in vivo toxicity of inhaled FAB1 Fab in powder formulation using the new formulation as described in Example 4 (TLTH, pH 5.5 with 3.14% histidine (w/w)), a 28-day good laboratory practice (GLP) toxicity study was set up and compared with an earlier toxicity study using TLTC, pH 6 with PS80.

28-Day Cyno GLP toxicity study results with FAB1 in TLTC, PH 6 with PS80. LLOQ=lower limit of quantification.

The table directly above shows the study results of a first 28-day toxicity study in which cynomolgus monkeys were treated with FAB1 reconstituted in TLTC at pH 6 with PS80. No adverse effects were seen on food consumption, body weights, clinical observations, clinical pathology, pulmonary function tests, ECGs, blood pressure, neurobehavioral assessments, or ophthalmology. There were also no adverse macroscopic or microscopic findings for all tissues including respiratory tract (i.e. lungs, trachea, larynx, oesophagus, salivary glands, lymph nodes, tongue, tonsils, soft palate) for animals treated with low dose levels (deposited dose level of 1.0 mg/kg/day).

However, at the higher doses of 2.3 (females only) and 7.1 mg/kg/day (both sexes) an increase of FAB1-related incidence and/or severity of lung perivascular (PV)/peribronchiolar (PB) mononuclear inflammatory cell (MIC) infiltrates (see FIG. 32). The no-observed-adverse-effect-level (NOAEL) was therefore 1 mg/kg/day in this first study using TLTC at pH 6+PS80 as excipient.

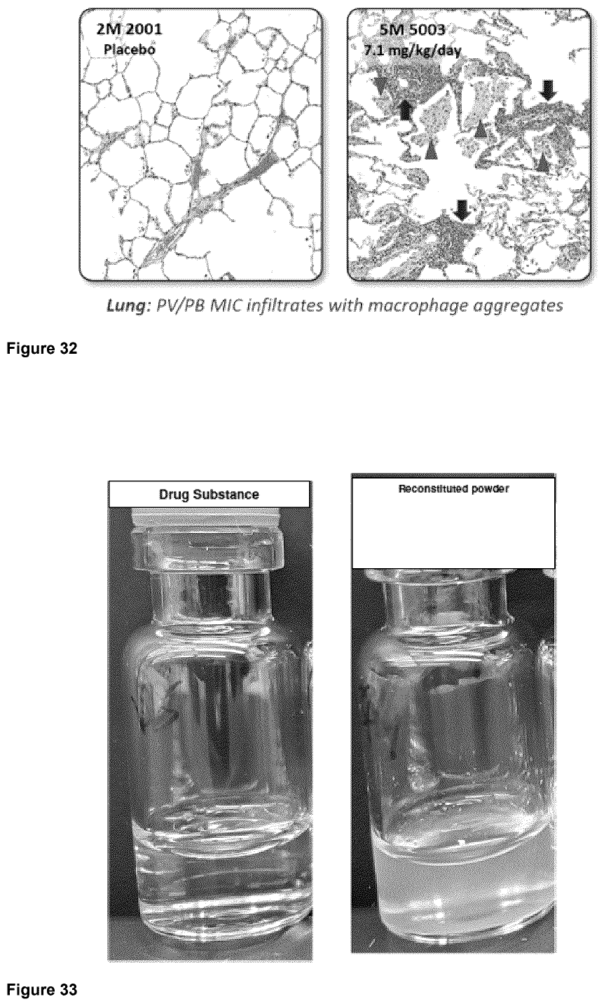

FIG. 33 further shows visible particles in the reconstituted formulation (FAB1 in TLTC at pH 6 with PS80) in water, which is a simplistic simulation of the expected reconstitution in epithelial lining fluid after lung deposition.

28-Day Cyno GLP toxicity study results with FAB1 in TLTH, pH 5.5 with 3.14% histidine.

| | | | | FAB1 | Target | Target Lung | |
|---|---|---|---|---|---|---|---|
| | | Exposure | Total Aerosol | Aerosol | Delivered | Deposited | Animals |
| | | Duration | Dose Level | Conc Level | Dose Level | Dose Level | Main |
| Group | Dose | (Minutes) | (mg/L) | (mg/L) | (mg/kg/d) | (mg/kg/d) | Study |
| 1 | Air | 60 | 0 | 0 | 0 | 0 | 3M + 3F |
| | Control | | | | | | |
| 2 | Placebo | 60 | 0.724 | LLOQ | 0 | 0 | 3M + 3F |
| | Control | | | | | | |
| 3 | Low Dose | 60 | NC (0.090) | 36 | 1.2 | 0.3 | 3M + 3F |
| 4 | Mid Dose | 60 | NC (0.303) | 121 | 4 | 1 | 3M + 3F |
| 5 | High Dose | 60 | 0.72 | 278 | 9.2 | 2.3 | 3M + 3F |

LLOQ = lower limit of quantification.

The table directly above shows the results of a second 28-day toxicity study in which cynomolgus monkeys were treated with TLTH, pH 5.5 with 3.14% histidine (w/w).

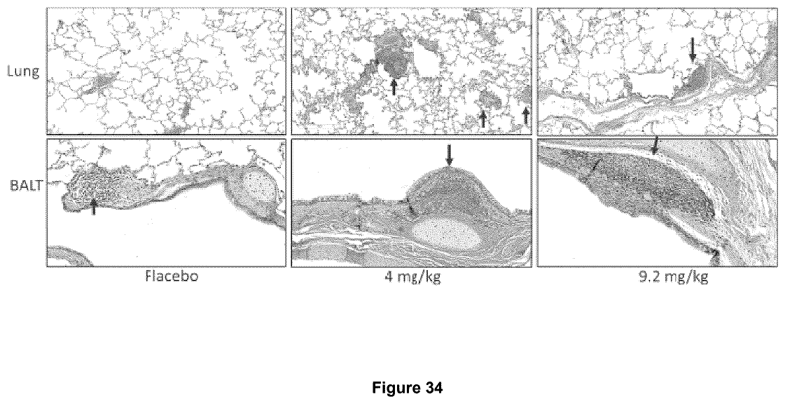

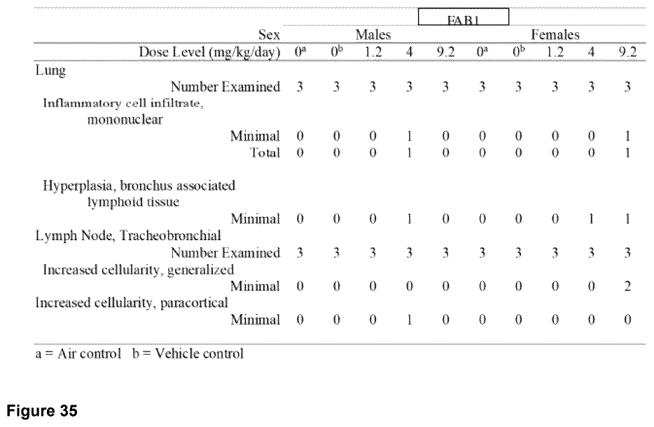

FIG. 34 shows representative images of FAB1-related lung pathology. Mononuclear cell (MNC) infiltrates in the lungs were minimal, for all tested doses. Microscopic findings were suggestive of a localised low-grade immune response to an inhaled foreign protein and were considered non-adverse (FIG. 35). The NOAEL is this study was 2.3 mg/kg deposited dose (as opposed to 1 mg/kg deposited dose for the first toxicity study).

Taken together, the above data show that the common microscopic finding associated with inhaled proteins in cynomolgus monkeys is mononuclear inflammatory cell (MIC) infiltrate in the lung. In the first 28-day toxicity study, PB/PB MIC infiltrates were observed, at a severity that was considered adverse and limited the NOAEL to 1 mg/kg. A TLTH formulation with pH 5.5 and 3.14% histidine (w/w) was associated with decreased in vitro aggregation and exhibited a more favorable toxicology profile (2.3 mg/kg NOAEL vs 1 mg/kg NOAEL) in cynomolgus monkeys compared with TLTC, pH 6 with PS80.

### Example 6-Phase I, Randomised, Blinded, Placebo-Controlled Study to Evaluate the Safety, Tolerability, Pharmacokinetics and Pharmacodynamics of FAB1 in Healthy Adult Subjects (Part A) and Adults with Asthma on Medium to High Dose Inhaled Corticosteroids and Long-Acting Beta-Agonists (Part B)

### Study Design

Part A of the study was a randomised, single-blinded, placebo-controlled study in male and female healthy volunteers to evaluate the safety, tolerability, PK, and immunogenicity of FAB1 by DPI administration (1 cohort in Sub Part A1 received IV FAB1). Part A consisted of 4 sub parts (A1, A2, A3, and A4). The overall design of Part A is presented in the following table:

Study Design - Part A

| Sub-parts | Population | Cohorts and treatments received |
|---|---|---|
| A1 | Healthy volunteers | 5 cohorts: SAD of 0.2 mg, 0.6 mg, 2 mg, 6 mg, and 16 mg of |
| | | inhaled FAB1 or placebo |
| | | 1 cohort: Single 5 mg IV dose of FAB1 or placebo |
| A2 | Healthy volunteers of | 2 cohorts (1 Chinese cohort, and 1 Japanese cohort): Single |
| | Chinese and Japanese | 16 mg dose of inhaled FAB1 or placebo |
| | ethnicity | |
| A3 | Healthy volunteers | 3 cohorts: MAD of 2 mg, 6 mg, and 16 mg of inhaled FAB1 or |
| | | placebo, once-daily for 14 days |
| A4 | Healthy volunteers of | 2 cohorts (1 Chinese cohort, and 1 Japanese cohort): highest |
| | Chinese and Japanese | dose in Sub-Part A3 of inhaled FAB1 or placebo, once-daily for |
| | ethnicity | 14 days. |

MAD, multiple ascending dose; SAD, single ascending dose.

The primary objectives for Part A were the safety and tolerability of inhaled FAB1, and the PK and safety of IV FAB1. Secondary objectives were the PK of inhaled FAB1 (including participants of Japanese and Chinese ethnicity), and the immunogenicity of FAB1 following single and multiple dose administration.

Part B of the study was a randomised, double-blinded, placebo-controlled study in male and female adults with asthma on a combination of medium to high dose ICS plus LABA medications. Patients were randomised to one of 3 inhaled dose levels (0.4 mg, 2 mg, and 8 mg) of FAB1 or placebo, once daily by dry powder inhaler (DPI) administration, for 28 days in a parallel-group design.

The predicted dose to man following inhaled administration of FAB1 was based on two elements: First, the predicted human PK profile for FAB1 systemically and in the lung tissue, secondly, identification of a target lung concentration based on clinical efficacy data for tezepelumab. The clinical PK profile for FAB1 was predicted using PK parameters which were allometrically scaled from cynomolgus monkeys. After inhaled administration, the average partition of FAB1 from lung to systemic circulation was estimated to be 2500 based on bronchial-alveolar lavage data from cynomolgus monkeys.

A target C_{trough} concentration in the lung was identified from a therapeutically efficacious systemic exposure of the systemic TSLP specific mAb (tezepelumab), with an assumed lung distribution coefficient from the serum. A calculated lung deposited dose of 1 mg (once daily) resulted in a C_{trough} concentration higher than the target concentration in lung tissue, corresponding to predicted average concentration (Cₐᵥₑ) in lung with 210 mg dose every 4 weeks of TSLP inhibiting systemic mAb, which has been proven to be efficacious in a Phase 3 study (Corren et al. N Engl J Med 2017:377: 936-946). Based on these assumptions, delivered doses of 0.4, 2 and 8 mg (once daily over 28 days) were proposed in Part B of the study, with a decrease in FeNO as the primary outcome.

The primary objective was the safety and tolerability of inhaled FAB1 in patients with asthma on medium/high dose ICS/LABA. Secondary objectives were the PK and immunogenicity of inhaled FAB1, and the abovementioned effect on FeNO of inhaled FAB1 versus placebo, following once daily administration for 28 days.

Exploratory objectives were to evaluate the effect of FAB1 in multiple doses on lung function in asthma and on asthma symptoms and rescue medication/reliever therapy/use. Lung function measures were assessed by change from baseline in pre-bronchodilator (pre-BD) FEV₁ and FVC and post-bronchodilator (post-BD) FEV₁ and FVC. Asthma symptom measures were assessed by change from baseline in weekly ACQ-6 score.

### For Inclusion in the Part B Study Patients had to Fulfil the Following Criteria:

1. Aged 18 to 75 years inclusive, with suitable veins for cannulation or repeated venipuncture.
2. Have a BMI between 18 and 35 kg/m² inclusive and weigh at least 45 kg.
3. Confirmed physician-led diagnosis of asthma for >6 months before the Screening Visit. Patients must be on a stable combination of LABA and ICS total daily dose >250 to 1000 μg fluticasone propionate DPI or equivalent for at least 1 month before the Screening Visit. If on asthma controller medications in addition to ICS plus LABA, the dose of the other asthma controller medications (xanthines, anticholinergics, leukotriene modifiers cromoglycate) must be stable for at least 4 weeks prior to screening visit.
4. Any of the following assessments within the last 10 years (documented in their medical history) to confirm variable airflow obstruction:
   (a) Variability between clinic visits: FEV1>12% and 200 mL.
   (b) Response to 4 weeks' anti-inflammatory therapy: FEV1>12% and 200 mL.
   (c) Exercise challenge test: FEV1 fall >10% and 200 mL.
   (d) Methacholine challenge test: FEV1≥20% fall at <8 mg/mL.
   (e) Indirect challenge test (saline or mannitol): FEV1≥15% fall.

### Or in the Screening Period:

(f) Variability between clinic visits: FEV1>12% and 200 mL.
(g) PEFR for 2 weeks during run-in: PEFR average daily variability >10%.
5. Pre-bronchodilator FEV1≥40% predicted at the Screening Visit in accordance with the ATS/ERS guidelines.
6. Have a FeNO of ≥30 ppb at the Screening Visit and ≥30 ppb at randomisation.
7. ACQ-6 score of ≥0.75 and ≤3.0 at screening.
8. During 7 consecutive days within Screening Period, demonstrates ≥65% adherence (˜4.5 days) to each of the following:
   (a) Twice daily home spirometry measurements
   (b) Twice daily entries in the eDiary (a compliant day comprises evening and subsequent morning diary entries).

### Results

### Study Populations

### Part A

A total of 96 healthy volunteers were randomized and treated in Part A of the study; 72 received FAB1 and 24 received placebo. All completed the study except for 1 participant in the 6 mg once daily FAB1 cohort who was lost to follow-up. The median age was 33.5 (range: 20 to 55) years old, and the majority were male (94.4%). The majority were White (93.5%), 5.2% were Black or African American, and 1.3% were of other race. Baseline characteristics (height, weight, body mass index) were balanced across the treatment groups.

### Part B

A total of 77 patients with asthma were randomized and treated in Part B of the study; 51 received FAB1 and 26 received placebo. All completed the study except for 1 patient in the 8 mg FAB1 cohort who withdrew consent. The median age was 52.0 (range: 21 to 75) years old, and 49.4% were male. The majority were White (93.5%), 5.2% were Black or African American, and 1.3% were of other race. Baseline characteristics (height, weight, body mass index) were balanced across the treatment groups.

### Pharmacokinetics

### Part A

Following inhalation of single doses (0.2 to 16 mg), time to maximum serum concentration of FAB1 (tmax) was observed at a median time of 8.0 to 11.0 hours. FAB1 serum concentrations declined in the dose range 2 mg to 16 mg with a geometric mean terminal half-life (t1/2λz) of 20.7 to 25.6 hours. Interparticipant variability was high as judged by geometric mean percent coefficient of variation (% CV) for maximum plasma (peak) drug concentration after a given number of doses (N) before steady state is reached (Cmax), area under the plasma concentration-time curve from time 0 to last quantifiable concentration (AUClast), and area under plasma concentration-time curve from time 0 to infinity (AUCinf). There were no major differences in estimated PK parameters between Chinese/Japanese participants and non-Asian participants.

Geometric mean (% CV) PK Parameters of FAB1 Following Single DPI Dose Administration in Healthy Volunteers (Parts A1 and A2)

| | | | | | Chinese and Japanese | |
|---|---|---|---|---|---|---|
| | SAD (Part A1) | | | | cohorts (Part A2) | |
| | Cohort 2 | | | | Chinese | Japanese |
| | FAB1 | Cohort 3 | Cohort 4 | Cohort 5 | cohort | cohort |
| | 0.6 mg | FAB1 | FAB1 | FAB1 | FAB1 | FAB1 |
| Parameter | once | 2 mg once | 6 mg once | 16 mg once | 16 mg once | 16 mg once |
| (Units) | (N = 6) | (N = 6) | (N = 6) | (N = 4) | (N = 4) | (N = 6) |
| AUC(0-24) | 17.66 | 26.88 | 95.17 | 273.0 | 230.4 | 262.9 |
| (h*ng/mL) | (27.0) | (33.7) | (26.1) | (68.3) | (28.6) | (68.4) |
| | [n = 4] | [n = 5] | [n = 6] | [n = 4] | [n = 4] | [n = 6] |
| AUClast | 11.02 | 31.94 | 158.2 | 568.2 | 412.9 | 501.3 |
| (h*ng/mL) | (104.5) | (36.2) | (42.1) | (86.6) | (30.5) | (57.7) |
| | [n = 6] | [n = 5] | [n = 6] | [n = 4] | [n = 4] | [n = 6] |
| AUCinf | NC | NC | 192.5 | 607.7 | 526.2 | 568.2 |
| (h*ng/mL) | | | (47.4) | (82.4) | (21.7) | (57.8) |
| | | | [n = 5 | [n = 4] | [n = 3] | [n = 6] |
| Cmax | 0.9844 | 1.321 | 5.721 | 14.57 | 11.96 | 15.05 |
| (ng/mL) | (24.4) | (49.3) | (36.6) | (67.3) | (31.7) | (70.8) |
| | [n = 6] | [n = 6] | [n = 6] | [n = 4] | [n = 4] | [n = 6] |
| tmax ^{a} (h) | 11.07 | 7.98 | 8.88 | 9.04 | 8.96 | 11.08 |
| | (10.08-12.10) | (5.98-10.03) | (4.05-24.00) | (8.00-10.13) | (3.07-10.35) | (4.00-24.15) |
| | [n = 6] | [n = 6] | [n = 6] | [n = 4] | [n = 4] | [n = 6] |
| t½λz | NC | 23.26 | 21.29 | 24.46 | 20.67 | 25.62 |
| (h) | | (46.7) | (46.7) | (27.4) | (24.6) | (25.3) |
| | | [n = 4] | [n = 5] | [n = 4] | [n = 3] | [n = 6] |

median (minimum − maximum). DPI, dry powder inhaler; N, number of participants in treatment group; n, number of participants included in analysis; NC, not calculated.
% CV, percent coefficient of variation; AUC(0-24), area under the plasma concentration-time curve from time 0 to 24 hours; AUClast, area under the plasma concentration-time curve from time 0 to last quantifiable concentration; Cmax, maximum plasma (peak) drug concentration after a given number of doses (N) before steady state is reached; t½λ, terminal elimination half-life; tmax, time to reach maximum concentration following drug administration of FAB1.

Following 14 days of daily doses of inhaled FAB1 at 2 mg, 6 mg and 16 mg, tmax of FAB1 was observed at a median time of 3.1 to 10.1 hours. At Day 14 serum concentrations declined with a geometric mean t1/2λz of 19.9 to 29.9 hrs in the dose range 2 mg to 16 mg. Upon repeated dosing, a 2- to 3-fold accumulation was observed in both AUC and Cmax and the systemic exposure generally increased in a dose-proportional manner. Interparticipant variability was high as judged by geometric mean % CV for Cmax, AUClast, and AUC (0-24). There were no major differences in estimated PK parameters in Chinese/Japanese participants and non-Asian participants following repeated dosing.

Geometric mean (% CV) PK Parameters of FAB1 Following Multiple DPI Dose Administration in Healthy Volunteers, Sub-Part A3

| | MAD (Part A3) | | | | | |
|---|---|---|---|---|---|---|
| | Cohort 1 | | Cohort 2 | | Cohort 3 | |
| | FAB1 | | FAB1 | | FAB1 | |
| | 2 mg | | 6 mg once | | 16 mg once | |
| | once daily | | daily | | daily | |
| Parameter | (N = 6) | | (N = 5) | | (N = 6) | |
| (Units) | Day 1 | Day 14 | Day 1 | Day 14 | Day 1 | Day 14 |
| AUC(0-24) | 37.24 | 77.58 | 100.2 | 280.9 | 265.2 | 773.4 |
| (h*ng/mL) | (36.8) | (40.4) | (54.7) | (41.3) | (58.9) | (50.1) |
| | [n = 6] | [n = 6] | [n = 5] | [n = 5] | [n = 6] | [n = 6] |
| AUClast | 36.84 | NC | 99.59 | NC | 264.6§ | NC |
| (h*ng/mL) | (37.0) | | (54.1) | | (58.3) | |
| | [n = 6] | | [n = 5] | | [n = 6] | |
| AUCinf | NC | NC | NC | NC | NC | NC |
| (h*ng/mL) | | | | | | |
| Cmax(ng/ | 2.161 | 4.413 | 5.816 | 16.72 | 15.47 | 41.60 |
| mL) | (38.5) | (33.2) | 65.5 | (46.0) | (57.7) | (47.7) |
| | [n = 6] | [n = 6] | [n = 5] | [n = 5] | [n = 6] | [n = 6] |
| Ctrough | NC | 3.517 | NC | 12.25 | NC | 34.98 |
| (ng/mL) | | (45.5) | | (49.8) | | (46.3) |
| | | [n = 6] | | [n = 5] | | [n = 6] |
| tmax ^{a} (h) | 5.04 | 7.06 | 6.02 | 6.08 | 10.10 | 5.00 |
| | (3.95-12.03) | (2.88-10.12) | (3.00-10.05) | (3.98-10.28) | (8.00-23.70) | (0.00-24.00) |
| | [n = 6] | [n = 6] | [n = 5] | [n = 5] | [n = 6] | [n = 6] |
| t½λz | 36.78 | 22.00 | 19.92 | 27.68 | NC | 27.85 |
| (h) | (47.8) | (42.9) | (28.2) | (35.3) | | (39.9) |
| | [n = 5] | [n = 6] | [n = 3] | [n = 5] | | [n = 6] |
| Rac AUC | NC | 2.083 | NC | 2.805 | NC | 2.916 |
| | | (33.1) | | (44.0) | | (30.5) |
| | | [n = 6] | | [n = 5] | | [n = 6] |
| Rac Cmax | NC | 2.042 | NC | 2.874 | NC | 2.689 |
| | | (39.6) | | (43.6) | | (37.4) |
| | | [n = 6] | | [n = 5] | | [n = 6] |

| | Chinese and Japanese | | | |
|---|---|---|---|---|
| | cohorts (Part A4) | | | |
| | Chinese cohort | | Japanese cohort | |
| | FAB1 16 mg | | FAB1 16 mg | |
| | once daily | | once daily | |
| Parameter | (N = 6) | | (N = 5) | |
| (Units) | Day 1 | Day 14 | Day 1 | Day 14 |
| AUC(0-24) | 246.7 | 600.9 | 247.5 | 484.2 |
| (h*ng/mL) | (72.9) | (70.4) | (56.0) | 59.0 |
| | [n = 6] | [n = 6] | [n = 5] | [n = 5] |
| AUClast | 245.4 | NC | 247.0 | NC |
| (h*ng/mL) | (73.3) | | (55.8) | |
| | [n = 6] | | [n = 5] | |
| AUCinf | NC | NC | NC | NC |
| (h*ng/mL) | | | | |
| Cmax(ng/ | 13.69 | 33.22 | 13.72 | 25.29 |
| mL) | (70.9) | (73.4) | (57.9) | (41.2) |
| | [n = 6] | [n = 6] | [n = 5] | [n = 5] |
| Ctrough | NC | 23.19 | NC | 18.61 |
| (ng/mL) | | (70.6) | | (66.7) |
| | | [n = 6] | | [n = 5] |
| tmax ^{a} (h) | 8.00 | 3.10 | 9.98 | 6.00 |
| | (3.00-11.97) | (1.50-9.83) | (3.10-23.70) | (3.00-12.02) |
| | [n = 6] | [n = 6] | [n = 5] | [n = 5] |
| t½λz | NC | 27.82 | NC | 29.88 |
| (h) | | (24.0) | | (11.7) |
| | | [n = 6] | | [n = 5] |
| Rac AUC | NC | 2.436 | NC | 1.956 |
| | | (57.9) | | (54.2) |
| | | [n = 6] | | [n = 5] |
| Rac Cmax | NC | 2.427 | NC | 1.843 |
| | | (52.1) | | (40.6) |
| | | [n = 6] | | [n = 5] |

^{a}median (minimum − maximum).
DPI, dry powder inhaler; N, number of participants in treatment group; n, number of participants included in analysis; NC, not calculated.

### Part B

The PK of FAB1 has been characterised in both healthy volunteers (Part A) and in patients with asthma in Part B. In healthy volunteers following 14 days of daily doses of inhaled FAB1 at 2 mg, 6 mg and 16 mg, and where a complete PK profile was generated, the observed Tmax of FAB1 was a median time of 5-7 hours (range 3-24 hours), with a geometric mean t1/2λz of 22-28 hours (range 14-45 hours) across the doses. In patients following 28 days of daily doses of inhaled FAB1 at 0.4 mg, 2 mg and 8 mg, the observed Tmax of FAB1 was a median time of 6 to 8 hours (range 0.25-24 hours). After the last dose at Day 28 serum concentrations had not declined sufficiently within the sampling period in all subjects to characterise the t1/2λz using non-compartmental analysis, however in 4/24 subjects a geometric mean t1/2λz of 31 hrs in the 8 mg once daily dose regimen could be estimated (range 23-48 hours). Upon repeated dosing, a 1- to 2.5-fold accumulation was observed in both AUC and Cmax and the systemic exposure generally increased in a dose-proportional manner. Interpatient variability was high as judged by geometric mean % CV for Cmax, AUClast, and AUC (0-24). Based on the estimated PK parameters, it can be concluded that the PK of FAB1 is similar between healthy volunteers (Part A) and patients with asthma (Part B).

Geometric mean (% CV) PK Parameters of FAB1 Following Multiple DPI Dose Administration in Patients with Asthma, Sub-Part B

| | Cohort 1 0.4 mg | | Cohort 2 2 mg | | Cohort 3 8 mg | |
|---|---|---|---|---|---|---|
| | once daily | | once daily | | once daily | |
| Parameter | (N = 13) | | (N = 13) | | (N = 24) | |
| (Units) | Day 1 | Day 28 | Day 1 | Day 28 | Day 1 | Day 28 |
| AUC(0-24) | NC | 24.05 | 36.17 | 75.66 | 86.49 | 238.0 |
| (h*ng/mL) | | (28.4) | (56.8) | (41.6) | (61.9) | (54.3) |
| | | [n = 7] | [n = 7] | [n = 11] | [n = 22] | [n = 21] |
| AUClast | 10.70 | NC | 35.98 | NC | 86.95 | NC |
| (h*ng/mL) | (150.2) | | (58.0) | | (62.2) | |
| | [n = 3] | | [n = 7] | | [n = 22] | |
| AUCinf (h*ng/mL) | NC | NC | NC | NC | NC | NC |
| Cmax (ng/mL) | 1.138 | 1.022 | 1.514 | 3.616 | 4.768 | 12.09 |
| | (77.1) | (33.8) | (83.9) | (62.6) | (64.5) | (53.0) |
| | [n = 4] | [n = 9] | [n = 10] | [n = 12] | [n = 22] | [n = 21] |
| tmax^{a} (h) ^{a} | 3.51 | 7.82 | 8.00 | 6.00 | 7.79 | 7.58 |
| | (2.00-23.78) | (0.25-24.00) | (3.92-24.00) | (0.25-24.00) | (2.00-26.50) | (0.00-24.00) |
| | [n = 4] | [n = 9] | [n = 10] | [n = 12] | [n = 22] | [n = 21] |
| t½λz (h) | NC | NC | NC | NC | 31.16 | 30.73 |
| | | | | | (60.1) | (14.0) |
| | | | | | [n = 3] | [n = 4] |
| Rac AUC | NC | 1.93 | NC | 2.56 | NC | 2.67 |
| | | (86.2) | | (70.6) | | (40.3) |
| Rac Cmax | NC | 0.8836 | NC | 2.554 | NC | 2.438 |
| | | (40.5) | | (63.1) | | (39.9) |
| | | [n = 4] | | [n = 10] | | [n = 20] |

### Immunogenicity

### Part A

In Part A, across the FAB1 groups the ADA prevalence (percentage of ADA evaluable participants who were ADA+ at any time) was 5.6% (4 of 71 evaluable participants), and the ADA incidence (percentage of ADA-evaluable participants who were TE-ADA+) was 1.4% (1 of 71 evaluable participants).

### Part B

Immunogenicity prevalence and incidence rates in Part B of the study were low (see the following table). One patient in the 2 mg cohort had a treatment-induced ADA-positive response on Day 28 of treatment, and 1 patient in the 8 mg cohort had a treatment-induced ADA-positive on Days 14 and 28 of treatment.

Anti-Drug Antibody results-Multiple Dose DPI in Patients with Asthma (Part B),

| | FAB1 | FAB1 | FAB1 | |
|---|---|---|---|---|
| | 0.4 mg | 2 mg | 8 mg | Pooled |
| | once daily | once daily | once daily | FAB1 |
| | (N = 13) | (N = 13) | (N = 25) | (N = 51) |
| | n (%) | n (%) | n (%) | n (%) |
| ADA prevalence ^{a} | 0 | 1/13 (7.7) | 1/25 (4.0) | 2/51 (3.9) |
| ADA incidence ^{b} | 0 | 1/13 (7.7) | 1/25 (4.0) | 2/51 (3.9) |

^{a} Percentage of ADA-evaluable patients who were ADA positive at any time.
^{b} Percentage of ADA-evaluable patients who were treatment-induced or treatment boosted ADA positive.

Percentages are based on the number of ADA-evaluable patients (patients with at least 1 ADA assessment).

### Pharmacodynamics

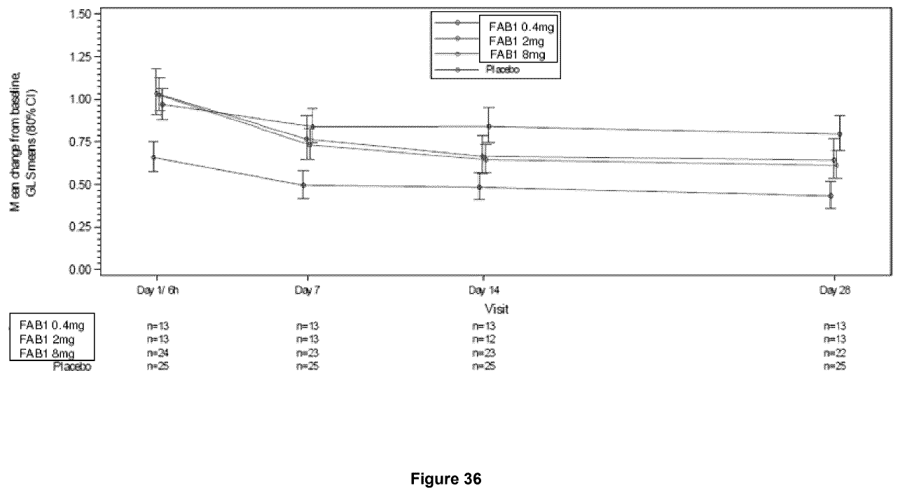

After 28 days of treatment, there was a numerical reduction in FeNO levels across all FAB1 cohorts (see the table below). FAB1 treatment reduced levels of FeNO as early as 6 hours post-dose in the 0.4 mg cohort and Day 7 in the 2 mg and 8 mg cohorts, and the reduction was sustained throughout 28 days (FIG. 36) (Error! Reference source not found). Although only the comparison between FAB1 8 mg and placebo cohorts was statistically powered, a statistically significant reduction in FeNO level was found in FAB1 8 mg and 0.4 mg cohorts. A 23% reduction (geometric mean ration 0.77; 1-sided p-value=0.0369) and 46% reduction (geometric mean ration 0.54; 1-sided p-value=0.0003) in FeNO levels were observed for 8 mg and 0.4 mg, respectively, compared to placebo.

Change from baseline in the FeNO level at Day 28—Part B (Pharmacodynamic Analysis Set)

The change from baseline in FeNO level was analysed using MMRM with treatment group,
baseline FeNO, visit, treatment-by-visit interaction as fixed effect and patient
as random-effect. Analyses was performed on the log-transformed FeNO data (change
from baseline and percentage change) to normalise the skewed distribution of this
endpoint and result back-transformed to linear scale. The within-patient correlation
was modeled using the unstructured covariance matrix. The Kenward-Roger approximation
was used to estimate denominator degrees of freedom. The analysis was performed
using only the OC without imputation of missing values. A REML method was used
for estimation. Treatment effect was estimated using contrasts of the LS means
on the correspondent treatment by-day interaction, along with 2-sided 80% CI and
1-sided test for the p-value corresponding to the between-treatment group difference.
One patient was excluded from due to incompatible FeNO data in CRF, and 2 patients
were excluded due to important protocol deviation.

| | Treatment | | | FAB1 versus placebo | | |
|---|---|---|---|---|---|---|
| | | Geometric | | Geometric | | |
| | n | LS means | 80% CI | mean ratio | 80% CI of ratio | p-value |
| FAB1 0.4 mg | 13 | 0.4322 | (0.3616, 0.5166) | 0.5437 | (0.4363, 0.6774) | 0.0003 |
| FAB1 2 mg | 13 | 0.6429 | (0.5376, 0.7689) | 0.8087 | (0.6488, 1.008) | 0.1083 |
| FAB1 8 mg | 22 | 0.6124 | (0.5354, 0.7005) | 0.7703 | (0.6396, 0.9278) | 0.0369 |

| | | | | |
|---|---|---|---|---|
| Placebo | 25 | 0.7950 | (0.6991, 0.9040) | NA |

CI, confidence interval; FeNO, fractional exhaled nitric oxide; LS, least squares; n, number of patients in a given category.

There was also a numerical improvement in lung function after 28 days of treatment as evidenced by clinic based pre-BD FEV1 compared to placebo (105 ml at week 4 at highest dose—see following table and FIG. 10)

Change (ml) from baseline in the clinic pre-BD FEV1 at Day 28—Part B

| | FEV₁ change | | | | |
|---|---|---|---|---|---|
| | from baseline | FEV₁ change | FEV₁ change | FEV₁ change | FEV₁ change |
| | compared to | from baseline | from baseline | from baseline | from baseline |
| | placebo (1 h | compared to | compared to | compared to | compared to |
| | PSOI) | placebo (D 2) | placebo (D 7) | placebo (D 14) | placebo (D 28) |
| FAB1 0.4 mg (n = 13) | −18 ml [0.43] | +84 ml [0.20] | +47 ml [0.34] | −27 ml [0.38] | −6 ml [0.48] |
| FAB1 2 mg (n = 12) | +190 ml [0.03] | +193 ml [0.03] | +111 ml [0.17] | +120 ml [0.11] | +9 ml [0.47] |
| FAB1 8 mg (n = 21) | +152 ml [0.03] | +270 ml [0.0009] | +151 ml [0.06] | +113 ml [0.09] | +105 ml [0.14] |

PSOI = post-start of inhalation;
n = number of subjects (D 28 figure);
numbers in square brackets denote p-value

In addition, there was as a numerical improvement in ACQ-6 symptoms (FIG. 11).

### Example 7—Population PK Model

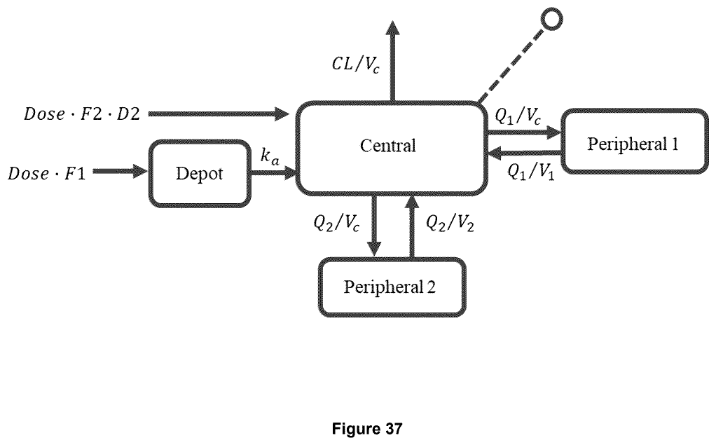

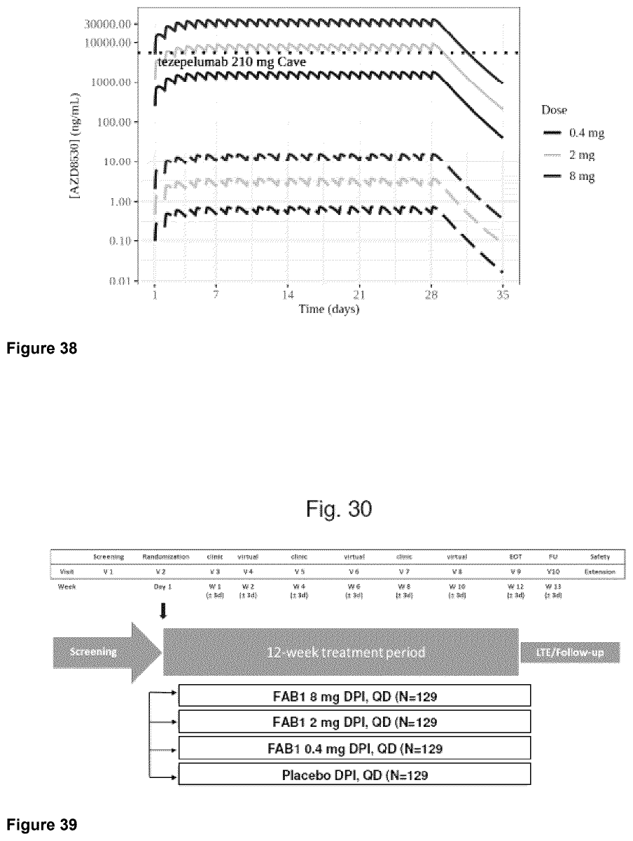

A population PK (popPK) model was developed to quantify the variability in observed clinical PK data and to understand any differences in population between those in Part 1A in healthy adult volunteers, and Part 1B in asthmatic adult patients on medium/high doses of inhaled corticosteroids/long acting beta2 agonists (NCT05110976). The popPK model had four compartments defined with a combined zero order and first order absorption of the administered dose in the lung, and observations defined by the dotted line in the serum of FAB1 (FIG. 37). Briefly, the IV and Part 1A single ascending dose data were used to estimate bioavailabilities for each absorption type, which were then fixed in the subsequent multiple ascending dose popPK model which included the individuals from Part 1A and Part 1B. The popPK model was simulated (n=1000 per dose) to obtain predictions and prediction intervals of serum concentrations (FIG. 38 dashed lines) and scaled to a predicted lung concentration (FIG. 38 solid lines) for Part 1B in patients at 0.4, 2 and 8 mg (once daily for 28 days).

### Dose Selection for Phase 2

The observed PK profile in Phase 1 (both in patients and healthy volunteers) was well aligned with the predicted clinical profile building confidence in the exposure assumption in relation to inhaled doses of FAB1. Predicted lung concentrations following the anticipated therapeutic dose of 2 mg were expected to be above the target level based on average exposure related to an efficacious dose of 210 mg QW4 of tezepelumab (FIG. 38). Further on a positive proof of mechanism with significant reduction of FeNO in asthmatic patients after inhaled administration of 8 mg QD of FAB1 provided the clinical relevance of TSLP inhibition in lung and confidence in dose range (0.4-8 mg) selected for Phase 2.

### Example 8—Phase 2b, Randomised, Double-Blind, Placebo-Controlled Dose Range-Finding Study to Assess Efficacy and Safety of 3 Dose Levels of Inhaled FAB1

This example describes a Phase 2b, randomised, double-Blind, placebo controlled dose range finding study to assess efficacy and safety of 3 dose levels of inhaled FAB1 (8 mg, 2 mg, 0.4 mg) given once daily via inhalation for 12 to 52 weeks in adults.

### Patient Population

The study will include adults (N=516, approximately) with documented physician-diagnosed asthma for a minimum of 12 months duration, a history of ≥1 severe exacerbation within the last 12 months. All participants will be symptomatic (asthma control questionnaire [ACQ] score ≥1.5) on background asthma therapy of medium or high dose ICS (as per GINA 2023 report) in combination with LABA±an additional non-biologic controller therapy (GINA step 4 or 5 therapy).

The target population includes severe asthma, similar to the tezepelumab clinical program, but expands to include moderate disease. Approximately 30% will have had 1 exacerbation in the last 12 months (defined as: asthma worsening which results in OCS use for ≥3 days, hospitalization or ER visit which results in systemic CS use) and approximately 70% of patients will have had ≥2 severe exacerbations within the last 12 months.

### Study Design

Eligible patients will be randomised 1:1:1:1 to FAB1 8 mg once daily, 2 mg once daily, 0.4 mg once daily or placebo. The range of doses in the Phase IIb study is based on results from the Phase I, Part b study, where these same 3 doses (8 mg, 2 mg, and 0.4 mg) were explored against placebo.

The study is of variable length with a 12-week treatment period and an optional safety extension of up to 52 weeks of total dosing. The safety extension component will end when the final patient enrolled to the study completes 12 weeks of treatment. The study design is provided in FIG. 39.

Primary and secondary endpoints are provided below.

### Objectives & Endpoints

| Objectives | Endpoints |
|---|---|
| Primary | |
| To evaluate the effect of FAB1 | Time to first CompEx asthma event |
| as compared to placebo on time | |
| to first CompEx asthma event in | |
| patients with uncontrolled | |
| moderate to severe asthma | |
| Secondary | |
| To evaluate the effect of FAB1 | Change from baseline of: |
| as compared to placebo on | 1. Pre-BD FVC: Weeks 1, 4 and 12 |
| lung function | 2. Post-BD FVC: Week 12 |
| | 3. Pre-BD FEV1: Weeks 1, 4 and 12 |
| | 4. Post-BD FEV1: Week 12 |
| | 5. Weekly mean morning PEF: Weeks 1, 4, 6, 8, 10 and 12 |
| | 6. Weekly mean evening PEF: Weeks 1, 4, 6, 8, 10 and 12 |
| To evaluate the effect of | Change from baseline: |
| FAB1 as compared to placebo | 1. Weekly mean asthma symptom diary score, Weeks 1, 4, |
| on asthma symptoms and control | 6, 8, 10 and 12 |
| | 2. ACQ-6: baseline, Weeks 2, 4, 8, 12 and over the |
| | treatment period |
| | 3. AQLQ: baseline, Weeks 4, 8 and 12 |
| | 4. SGRQ: baseline and week 12 |
| To evaluate the effect of | Change from baseline: |
| FAB1 as compared with placebo | 1. FeNO: Weeks 1, 4, 8 and 12 |
| on asthma-related biomarkers | 2. Blood eosinophils, Weeks 1, 4, 8 and 12 |
| | 3. IgE, Weeks 1, 4, 8 and 12 |
| To evaluate the PK of | FAB1 and anti-drug antibody (ADA) plasma concentrations: |
| FAB1 and ADA | pre-dose at baseline, Weeks 4, 8 and 12 |

### Primary Endpoint

The CompEx Asthma is a composite endpoint that allows evaluation of treatment effect on exacerbation involving fewer participants compared with severe exacerbations. There are two main types of CompEx Asthma events:
Severe exacerbations of asthma
Diary-based (objective deterioration)

### Severe Exacerbations of Asthma CompEx Events

Asthma exacerbations will be evaluated by the investigator at each visit. Severe exacerbations are defined as those episodes that lead to hospitalisation, emergency room visit, and/or treatment with oral glucocorticosteroid as detailed below:
Inpatient hospitalization: an admission to an inpatient facility and/or evaluation and treatment in healthcare facility for ≥24 hours due to asthma.
Emergency room or urgent care visit: evaluation and treatment for <24 hours in an emergency department or urgent care centre due to asthma required systemic corticosteroids.
Use of a temporary bolus/burst of systemic corticosteroids (or a temporary increase in stable OCS background dose) for at least 3 consecutive days to treat symptoms of asthma worsening; a single depo-injectable dose of corticosteroids will be considered equivalent to a 3-day bolus/burst of systemic corticosteroids.

### Diary-Based CompEx Events

Diary-based CompEx events are based on patient-reported deteriorations in three e-Diary variables, captured twice daily (morning and evening). This combination results in 6 different e-Diary variables.

Diary-based CompEx events are defined by threshold and slope criteria using the following Morning/Evening e-Diary variables:
Peak expiration flow (PEF-morning [PEFm] and evening [PEFe])
PEF (L/min) is a home spirometry measure. The capture of PEF follows standardized procedures. During data collection, all required attempts (usually three) are recorded. Only the best of the three attempts (max PEF) is included in the diary dataset and should be used in calculating CompEx events.
PEFm measurements are conducted at home by the patient, with the exception of site visit days. On a site visit day, the patient performs PEF assessment on-site, and the home PEFm data might not be available. PEFm cannot be imputed with site PEF measurements from on-site visit days (this is because PEFm is patient-reported data and site PEF is investigator-reported data and these two data sources cannot be used interchangeably in CompEx calculations).
Symptom score (0-3) (morning [Sm] and evening [Se])
Asthma symptom scores during night-time and day-time will be assessed by the patient each morning and evening according to the following scoring system:
0: You have no asthma symptoms.
1: You are aware of your asthma symptoms, but you can easily tolerate the symptoms.
2: Your asthma is causing you enough discomfort to cause problems with normal activities (or with sleep).
3: You are unable to do your normal activities (or to sleep) because of your asthma.
Use of rescue medication (number of doses) (morning [Rm] and evening [Re])
Rescue medication use is measured by the number of puffs taken of SABA used during the study.
The number of doses of rescue medication is defined as the number of puffs of inhaler recorded in the morning (for preceding night) and evening (for preceding day), respectively. If a nebulizer is used in a study, the number of doses of reliever medication use is defined as the number of puffs of inhaler plus twice the number of nebulizer applications.

### Determination of Diary-Based (Objective Deterioration) CompEx Events

The e-Diary events are based on deteriorations in the e-Diary variables PEFm, PEFe, Sm, Se, Rm and Re as defined above. Diary-based CompEx Asthma events can be of two types based on:
Threshold criteria
Threshold and slope criteria.

A participant will be considered to have a CompEx event during the planned treatment period if the participant has one or both of the following:
An objective deterioration, which is defined as either the threshold criterion or
The slope criterion (or both), as defined below, being met for ≥2 consecutive days.

For this purpose, “2 consecutive days” means strictly the same 2 consecutive days when assessing multiple requirements within those days. For the e-Diary data (which is captured twice during the day), one day will be defined by the morning/evening pairing for consistency with published precedent for the CompEx endpoint. (Note: other e-Diary endpoints in this study will use an evening/morning pairing to define one day.) The morning e-Diary recordings captured on the first day of treatment will not be included in the calculation of the CompEx endpoint.

### Baseline for Diary-Based Variables

Before threshold and slope criteria are assessed, baseline values need to be calculated for each of the six diary-based variables: PEFm, PEFe, Sm, Se, Rm and Re. Baseline values will be calculated for each individual patient as the average of the variable during the last ten days of the Run-in Period (days −10 to −1 with day −1 denoting the day before randomisation). In the event that less than 10 days of data is available, at least 5 days of data is required to calculate the baseline values.

CompEx Asthma events cannot be calculated for participants with missing baseline diary-based variables.

### Threshold Criteria CompEx Asthma Event:

a. PEFm or PEFe≥15% decrease from baseline in either morning or evening home-based PEF, and at least one of the following:
b. Rm or Re≥1.5 doses increase from baseline in rescue medication in either the morning (for preceding night) or evening (for preceding day)
c. Sm or Se≥1 score increase from baseline in symptom score or achieving the absolute maximal symptom score (3), in either the morning or evening. This means the criterion is also met when the value is at the highest on the symptom score 3.
   For (b), the number of doses of rescue medication is defined as the number of puffs of inhaler recorded in the morning and evening, respectively.

Assessment of the threshold criteria in any rolling 2-day consecutive period will be based on the available data during that period. The threshold criteria can be met with non-missing values for fewer than the six variables specified above, provided those non-missing values meet the criteria. In other words, this gives a total of eight variable combinations: PEFm-Rm, PEFm-Re, PEFe-Rm, PEFe-Re and PEFm-Sm, PEFm-Se, PEFe-Sm, and PEFe-Se, where the deterioration criteria need to be fulfilled for both variables in at least one combination for at least 2 consecutive days.

### Threshold and Slope Criteria CompEx Asthma Event:

A threshold and slope criteria CompEx Asthma event is when: (a), (b) or (c) of the threshold criteria above is met for at least 2 consecutive days and the regression slope requirement over the preceding 5 days is also met. Note that a CompEx event is never based on slope criteria only.

The regression slope requirement in the preceding 5 days is that all of the following are met:
PEFm slope≤−3%/day
PEFe slope≤−3%/day
Rm slope ≥0.3 doses/day
Re slope ≥0.3 doses/day
Sm slope ≥0.2 score/day
Se slope ≥0.2 score/day.

In all of the above cases, the regression slope is the point estimate of the slope obtained from a linear regression of the absolute values of each of the six variables separately against day number, with no other variables included in the model.

For PEFm and PEFe, the regression slope thus obtained will first also be divided by the baseline PEFm and PEFe value and multiplied by 100 respectively before applying the above criteria.

A regression slope will be calculated provided there are at least two non-missing values in the required 5 days. If one or more of the six variables above does not have at least two non-missing values in the required 5 days, then the slope requirement cannot be met.

### Duration of Diary-Based CompEx Asthma Events

The start date of a CompEx Asthma event is defined as the earliest of the exacerbation or objective deterioration start dates which meets the definition. Objective deterioration start date is defined as the earliest Day 1 from any series of rolling 2 consecutive days which first qualifies using either the threshold or slope criteria.

The end date of a CompEx event is defined as the latest of the exacerbation or objective deterioration end dates which meets the definition. Objective deterioration end date is defined as the latest from any series of rolling 2 consecutive days which last qualifies using either the threshold or slope criteria.

Whether or not diary-based CompEx criteria are met, is evaluated by a rolling window, with each pair of two consecutive days evaluated for fulfilment of the criteria. This also applies if different consecutive days fulfil different types of criteria (threshold only or threshold and slope).

### Combining CompEx Asthma Events

If the end date of the first CompEx event and the start date of the second CompEx event are less than 7 days apart for any participant, then these will be counted as one CompEx event.

### Example 9—Study to Compare an Inhaler of the Present Disclosure with an RS01 Monodose Inhaler

This study compares the administration of formulations using an inhaler as disclosed herein with an RS01 monodose inhaler, a dry powder inhaler that is conventional in the art.

### Experimental

The tested formulations were produced by spray drying and are shown in the table below.

| FAB1 | Trileucine | Leucine | Trehalose | Histidine |
|---|---|---|---|---|
| (wt. %) | (wt. %) | (wt. %) | (wt. %) | (wt. %) |
| 1 | 2.0 | 10.5 | 83.3 | 3.2 |
| 20 | 2.0 | 10.5 | 64.3 | 3.2 |

FAB1: an antibody fragment of the present disclosure.

20 mg of each formulation was filled into a hydroxypropylmethyl cellulose capsule. A Next Generation Impactor (NGI), i.e. a high-performance cascade impactor, was used to measure the aerodynamic particle size distribution of the formulations when dispensed from the capsule using the different inhalers. The NGI was operated at a constant flow rate with a 4.0 kPa pressure drop according to United States Pharmacopeia (USP)<601> Apparatus 6. The delivered dose uniformity (DDU) was measured with a dosage unit sampling apparatus according to USP<601>, Apparatus B, Product Performance Tests-Nasal and Inhalation Aerosols, Sprays, and Powders.

The following formulation and inhaler device combinations were tested:

| | | Replicates | Replicates | |
|---|---|---|---|---|
| | Inhaler | per test | per test | |
| Formulation | device | (NGI) | (DDU) | Protocol |
| 1% FAB1 (0.2 mg | Monodose | 5 | 10 | RFA-22- |
| per capsule) | | | | NC065 |
| 1% FAB1 (0.2 mg | Inhaler of the | 5 | 10 | RFA-22- |
| per capsule) | disclosure | | | NC065 |
| 20% FAB1 (4 mg | Monodose | 6 | 10 | RFA-22- |
| per capsule) | | | | NC049 |
| 20% FAB1 (4 mg | Inhaler of the | 5 | 10 | RFA-22- |
| per capsule) | disclosure | | | NC065 |

### Results

The results of the tests using the NGI are shown in the following table:

| | 1% FAB1 | 1% FAB1 | 20% FAB1 | 20% FAB1 |
|---|---|---|---|---|
| | (0.2 mg per | (0.2 mg per | (4 mg per | (4 mg per |
| Formulation | capsule) | capsule) | capsule) | capsule) |
| Device | Monodose | Inhaler of the disclosure | Monodose | Inhaler of the disclosure |
| Average Delivered Dose (mg) | 0.16 | 0.16 | 3.4 | 3.4 |
| Average Fine Particle Fraction <5 μm (%) | 78 | 80 | 76 | 80 |
| Average Mass Median Aerodynamic Diameter (μm) | 3.05 | 3.00 | 3.14 | 2.92 |

It can be seen that the average delivered dose of the active ingredient for each formulation was the same irrespective of which inhaler was used to dispense the formulations. The average delivered dose for each formulation was at least 80% which is high for dry powder formulations. For both formulations, the average fine particle fraction (FPF) was higher with the inhaler of the disclosure than the RS01 monodose inhaler. The average mass median aerodynamic diameter of the particles is lower for both formulations where the inhaler of the disclosure was used.

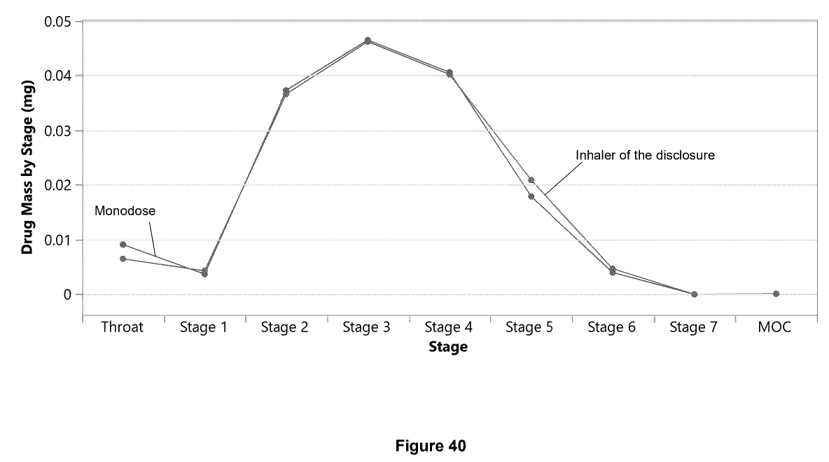

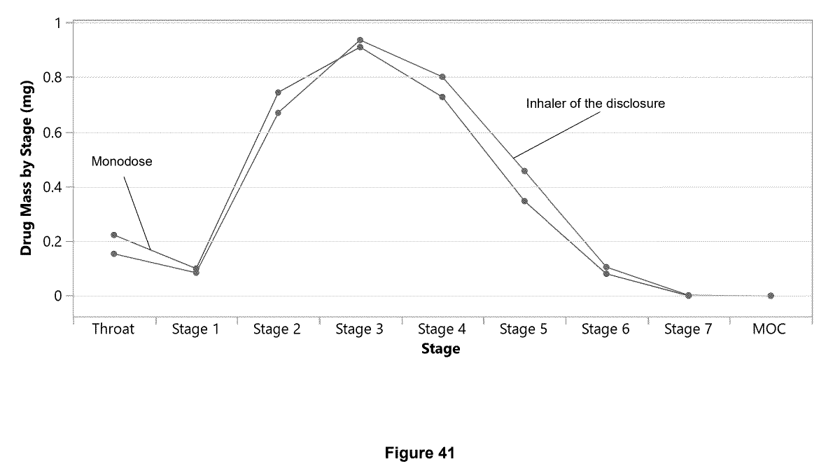

FIGS. 40 and 41 show the results of the testing performed with the NGI for each formulation with both the inhaler of the disclosure and the RS01 monodose inhaler. It can be seen that, for both formulations, a greater mass of the formulation reaches the later stages of the cascade impactor when the inhaler of the disclosure is used. Since it is the smaller particles that reach the later stages of the impactor (the stages on the right-hand side of the graphs), this means that the inhaler of the disclosure is dispensing the dry powder formulations in a finer particulate form. These results are therefore consistent with the FPF and average mass median aerodynamic diameter data shown in the table above.

The formation of finer particles is associated with improved delivery of the formulation to the lungs of the subject. The data thus demonstrates that improved delivery of the dry powder formulation to the lungs may be achieved by using a device of the present disclosure.

### Example 10—Flow Dependence Studies

This study investigates the effect of different inhalation pressures on the performance of an inhaler of the present disclosure.

### Experimental

The capsules used in the study contained the dry powder formulations shown in the table below:

| | 8.0 mg FAB1 formulation | 0.2 mg FAB1 formulation |
|---|---|---|
| FAB1 (mg) | 8.00 | 0.20 |
| Histidine (mg) | 0.63 | 0.63 |
| Trehalose (mg) | 8.87 | 16.67 |
| Trileucine (mg) | 0.40 | 0.40 |
| Leucine (mg) | 2.10 | 2.10 |
| Total (mg) | 20.00 | 20.00 |

Performance was tested at 2.0 kPa (representative of a relatively weak inhalation) and 6.0 kPa (representative of a relatively strong inhalation), as opposed to the 4.0 kPa that is used in Example 9. The following tests were conducted:
Delivered dose uniformity was measured with a dosage unit sampling apparatus according to USP<601>, Apparatus B. 10 replicates of the experiment were carried out for each formulation at both pressures.
The fine particle fraction (<5 μm) was measured with a Next Generation Impactor (NGI) according to USP<601>, Apparatus 6. Five replicates of the experiment were carried out for each formulation at both pressures.

### Results

The average delivered dose for each experiment is shown in the table below:

| | 2 kPa | 6 kPa |
|---|---|---|
| 0.2 mg formulation | 80% | 82% |
| 8 mg formulation | 82% | 84% |

The data demonstrates that the delivered dose remains at over 80% at both pressures and for both drug formulations.

The average fine particle fraction for each experiment is shown in the table below:

| | 2 kPa | 6 kPa |
|---|---|---|
| 0.2 mg formulation | 87% | 83% |
| 8 mg formulation | 85% | 81% |

The data demonstrates that the average FPF was over 80% at both pressures and for both drug formulations. Taken together, the data in this study indicates that the inhaler of the disclosure exhibits a good level of flow independence in its performance.

### Example 11—Intended Use Life Tests

This study investigates the performance of an inhaler of the present disclosure when used to administer multiple doses of a dry powder formulation over multiple days.

### Experiments

The dry powder formulations that were used in the study are the same as those used in Example 10.

One capsule per day was administered for 14 days from the same inhaler, although two doses per day were administered on Fridays and Mondays to represent the Saturday and Sunday doses. Delivered dose was measured for both the 0.2 mg and 8 mg FAB1 formulations with five replicates of the experiment, while fine particle fraction was measured for the 8 mg FAB1 formulation with three replicates of the experiment. Test methods are as described in Example 9.

### Results

The results of the delivered dose experiments are shown in the following table:

| | |
|---|---|
| Dose (day | Average delivered dose (%) |

| of testing) | 0.2 mg formulation | 8 mg formulation |
|---|---|---|
| 1 | 82% | 86% |
| 2 | 82% | 88% |
| 4 | 80% | 87% |
| 6 | 80% | 84% |
| 7 | 79% | 83% |
| 8 | 80% | 83% |
| 9 | 84% | 83% |
| 11 | 79% | 80% |
| 13 | 83% | 82% |
| 14 | 83% | 83% |

The results of the fine particle experiments are shown in the following table:

| Dose (day of testing) | 8 mg formulation |
|---|---|
| 1 | 82% |
| 6 | 81% |
| 8 | 80% |
| 14 | 80% |

The data shows that the delivered dose remained stable throughout the 14 day test period for both of the tested formulations. In the fine particle fraction testing, the % of fine particles remained stable for the duration of the 14 day period. This study therefore demonstrates that the performance of the inhaler of the disclosure is stable over an extended use period.

### Example 12—Delivered Dose Total Recovery

This study investigates the amount of dry powder formulation that is retained in an inhaler of the present disclosure after administration, as compared to an RS01 monodose inhaler.

### Experimental

The formulation that was used in the experiment is the same as the FAB1 1% formulation described in Example 9. Capsules containing the dry powder formulation were administered from the inhalers and the amount of FAB1 that was retained in the inhaler was measured solvent extracting the residual FAB1 from the inhaler.

### Results

The results of the experiment are shown in the table below:

| | | Inhaler of the |
|---|---|---|
| | Monodose | disclosure |
| Average retained in inhaler (mg) | 0.01 | 0.00 |

The results show that the inhaler of the disclosure exhibits excellent powder deposition resistance. This is particularly important for inhalers that are used to administer multiple doses, since deposit build up over time can notably reduce inhaler performance.

1. A preloaded inhaler comprising a spin chamber, the spin chamber comprising:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air to travel therethrough, the at least one curved inlet channel defining a curved recess and comprising a tangential section and a funnel section,
wherein
at least a portion of the tangential section is substantially tangential to the curved wall of the primary recess;
the tangential section is connected at a first end to an air inlet on an exterior surface of the spin chamber and at a second end to a first end of the funnel section, wherein the air inlet is configured to allow air to enter therethrough into the spin chamber; and
the funnel section curves toward the primary recess and is connected at a second end to an entry point configured to allow air to enter therethrough into the primary recess, wherein the funnel section is downstream from the tangential section;
wherein the curved inlet channel is separated from the primary recess along a majority of its length by the curved wall of the primary recess; and
wherein the preloaded inhaler comprises a capsule held in the spin chamber, the capsule containing a dry powder formulation which comprises an antigen binding fragment of an anti-thymic stromal lymphopoietin (TSLP) antibody.

2. The inhaler of claim 1, wherein:
the spin chamber has a longitudinal axis extending from a top of the spin chamber, down through the primary and secondary recesses, to a bottom of the spin chamber;
the spin chamber comprises a top surface located at the top of the spin chamber with respect to the longitudinal axis;
the primary recess is proximate to the top of the spin chamber along the longitudinal axis, and the secondary recess is proximate to the bottom of the spin chamber along the longitudinal axis;
the bottom surface of the primary recess faces the top of the inhaler with respect to the longitudinal axis; and
the spin chamber is configured so that in use air flows in from the air inlet, through the at least one curved inlet channel, through the primary recess and out through an outlet of the inhaler.

3-15. (canceled)

16. The inhaler of claim 1, wherein the formulation comprises microparticles which comprise the antigen binding fragment; and
wherein the microparticles are spray dried microparticles.

17. The inhaler of claim 3, wherein the dry powder formulation further comprises leucine, trileucine, or a combination thereof;
and wherein the mass ratio of leucine:trileucine in the formulation is from 1:1 to 12:1; and optionally from 3:1 to about 7:1.

18-20. (canceled)

21. The inhaler of claim 1, wherein the dry powder formulation comprises the antigen binding fragment in an amount of from 1% to 60% by weight, or in an amount of from 1% to 45% by weight of the dry powder formulation.

22. The inhaler of claim 1, wherein the antigen binding fragment comprises:
a. a HCDR1 comprising or consisting of the amino acid sequence of SEQ ID NO: 1;
b. a HCDR2 comprising or consisting of the amino acid sequence of SEQ ID NO: 2;
c. a HCDR3 comprising or consisting of the amino acid sequence of SEQ ID NO: 3;
d. a LCDR1 comprising or consisting of the amino acid sequence of SEQ ID NO: 5;
e. a LCDR2 comprising or consisting of the amino acid sequence of SEQ ID NO: 6; and
f. a LCDR3 comprising or consisting of the amino acid sequence of SEQ ID NO: 7.

23. (canceled)

24. The inhaler of claim 1, wherein the antigen binding fragment is a Fab derived from an IgG1 antibody, wherein the antigen binding fragment comprises a heavy chain comprising the sequence set forth in SEQ ID NO: 28 and a light chain comprising the sequence set forth in SEQ ID NO: 29.

25. The inhaler of claim 24, wherein the antigen binding fragment consists of the sequence set forth in SEQ ID NO: 28 and a light chain consists of the sequence set forth in SEQ ID NO: 29.

26-35. (canceled)

36. The inhaler of claim 1, wherein the dry powder formulation comprises:
(a) 10.5% of leucine; 2% of trileucine; 0.55% of L-histidine; 2.59% of L-histidine HCl; 2% of the antigen binding fragment; and 82.36% trehalose, by weight of the dry powder formulation;
(b) 10.5% of leucine; 2% of trileucine; 0.55% of L-histidine; 2.59% of L-histidine HCl; 10% of the antigen binding fragment; and 74.36% trehalose, by weight of the dry powder formulation; or
(c) 10.5% of leucine; 2% of trileucine; 0.55% of L-histidine; 2.59% of L-histidine HCl; 40% of the antigen binding fragment; and 44.36% trehalose, by weight of the dry powder formulation.

37. The inhaler of claim 1, wherein the dry powder formulation has a compressed bulk density of about 0.4-1.0 g/cm³.

38. The inhaler of claim 1, wherein the dry powder formulation, following reconstitution, has (i) a number of sub-visible particles between 5 μm to 200 μm of less than 2.5×10⁴/ml, or less than 0.5×10⁴/ml; (ii) a number of sub-visible particles between 10 μm to 200 μm is of less than 1×10⁴/ml, or less than about 0.2×10⁴/ml; or
(iii) a number of sub-visible particles between 25 μm to 200 μm of less than about 2×10³/ml, or less than about 0.2×10³/ml.

39-43. (canceled)

44. A method of treating a TSLP-related condition in a subject in need thereof, the method comprising administering a dry powder formulation comprising an antigen binding fragment of an anti-thymic stromal lymphopoietin (TSLP) antibody to the subject, wherein the formulation is administered from a capsule using a preloaded inhaler which comprises the capsule, wherein the preloaded inhaler is as defined in claim 1.

45-46. (canceled)

47. The method of claim 44 wherein the TSLP-related condition is asthma, and wherein the dry powder formulation is administered by the inhaler at a dose of from 0.4 mg to 8 mg of the antigen binding fragment per dose.

48-50. (canceled)

51. The method of claim 44 wherein the TSLP-related condition is asthma, and wherein the subject is co-administered a background therapy; optionally, wherein the subject is already receiving the background therapy prior to the treatment.

52. The method of claim 13, wherein the background therapy is selected from: inhaled corticosteroids;
leukotriene modifiers; long-acting beta agonists (LABAs); long-acting muscarinic antagonists (LAMAs); a combination therapy of fluticasone and salmeterol, budesonide and formoterol, mometasone and formoterol, or fluticasone and vilanterol; theophylline; short-acting beta agonists (SABAs); ipratropium; or a combination of ipratropium and albuterol or ipratropium and an oral corticosteroid.

53. A kit comprising:
(i) an unloaded inhaler comprising a spin chamber, the spin chamber comprising:
a primary recess configured to receive air to mix with contents of a capsule, the primary recess having a curved wall configured to allow rotation of the capsule;
a secondary recess configured to hold the capsule, the secondary recess located within a bottom surface of the primary recess; and
at least one curved inlet channel configured to allow air to travel therethrough, the at least one curved inlet channel defining a curved recess and comprising a tangential section and a funnel section,
wherein
at least a portion of the tangential section is substantially tangential to the curved wall of the primary recess;
the tangential section is connected at a first end to an air inlet on an exterior surface of the spin chamber and at a second end to a first end of the funnel section, wherein the air inlet is configured to allow air to enter therethrough into the spin chamber; and
the funnel section curves toward the primary recess and is connected at a second end to an entry point configured to allow air to enter therethrough into the primary recess, wherein the funnel section is downstream from the tangential section;
wherein the curved inlet channel is separated from the primary recess along a majority of its length by the curved wall of the primary recess; and
(ii) one or more capsules for loading into the spin chamber of the inhaler, wherein the one or more capsules contain a dry powder formulation which comprises an antigen binding fragment of an anti-thymic stromal lymphopoietin (TSLP) antibody.

54. (canceled)

55. The kit of claim 53, wherein the dry powder formulation comprises:
(a) from 8% to 12% of leucine; from 1% to 3% of trileucine; from 1% to 5% of a histidine buffer; from 1% to 5% of the antigen binding fragment; and from 75% to 85% of trehalose, by weight of the dry powder formulation;
(b) from 8% to 12% of leucine; from 1% to 3% of trileucine; from 1% to 5% of a histidine buffer; from 5% to 15% of the antigen binding fragment; and from 65% to 80% of trehalose, by weight of the dry powder formulation; or
(c) from 8% to 12% of leucine; from 1% to 3% of trileucine; from 1% to 5% of a histidine buffer; from 30% to 50% of the antigen binding fragment; and from 40% to 50% of trehalose, by weight of the dry powder formulation.

56. The kit of claim 53, wherein the antigen binding fragment comprises a heavy chain comprising or consisting of the sequence set forth in SEQ ID NO: 28 and a light chain comprising of consisting of the sequence set forth in SEQ ID NO: 29.

57. The kit of claim 53 wherein the dry powder formulation comprises:
(a) 10.5% of leucine; 2% of trileucine; 0.55% of L-histidine; 2.59% of L-histidine HCl; 2% of the antigen binding fragment; and 82.36% trehalose, by weight of the dry powder formulation;
(b) 10.5% of leucine; 2% of trileucine; 0.55% of L-histidine; 2.59% of L-histidine HCl; 10% of the antigen binding fragment; and 74.36% trehalose, by weight of the dry powder formulation; or
(c) 10.5% of leucine; 2% of trileucine; 0.55% of L-histidine; 2.59% of L-histidine HCl; 40% of the antigen binding fragment; and 44.36% trehalose, by weight of the dry powder formulation.

58-59. (canceled)


SEQUENCE LISTING (31 sequences)

SEQ ID NO: 1 - protein, 5 aa, synthetic construct
TYGMH

SEQ ID NO: 2 - protein, 17 aa, synthetic construct
VIWYDGSNKHYADSVKG

SEQ ID NO: 3 - protein, 13 aa, synthetic construct
APQWELVHEAFDI

SEQ ID NO: 4 - protein, 122 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 5 - protein, 11 aa, synthetic construct
GGNNLGSKSVH

SEQ ID NO: 6 - protein, 7 aa, synthetic construct
DDSDRPS

SEQ ID NO: 7 - protein, 11 aa, synthetic construct
QVWDSSSDHVV

SEQ ID NO: 8 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 9 - DNA, 366 bp, synthetic construct
  source 1..366: mol_type = other DNA
cagatgcagttggttgaatctggtggcggcgtggtgcagcctggcagatctctgagactgtcttgtgccgcctccggcttcaccttcagaacctacggaatgcactgggtccgacaggcccctggcaaaggattggaatgggtcgccgtgatttggtacgacggctccaacaagcactacgccgactccgtgaagggcagattcaccatcaccagagacaactccaagaacaccctgaacctgcagatgaactccctgagagccgaggacaccgccgtgtactattgtgctagagcccctcagtgggaactcgtgcatgaggcctttgacatctggggccagggaacaatggtcaccgtctcctca

SEQ ID NO: 10 - DNA, 324 bp, synthetic construct
  source 1..324: mol_type = other DNA
tcatatgttcttacacaaccaccgtcggtttcggttgctccaggacaaacagctcgaattacatgcggaggaaacaacctcggatcgaagtcggttcactggtatcaacaaaagccaggacaagctccagttctcgtggtgtacgatgattcagatcgaccatcatggatcccagagcgattctcaggatcaaactcgggaaatactgccacgctcacaatttcacgcggagaagcgggagatgaagctgattactattgccaagtgtgggactcgtcgtcagatcatgttgttttcggaggtggaacaaagctcacagtgctc

SEQ ID NO: 11 - protein, 11 aa, synthetic construct
GGNNIGSKSVH

SEQ ID NO: 12 - protein, 108 aa, synthetic construct
SYVLTQPPSVSVAPGQTARITCGGNNIGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 11 aa, synthetic construct
GGNNVGSKSVH

SEQ ID NO: 14 - protein, 108 aa, synthetic construct
SYVLTQPPSVSVAPGQTARITCGGNNVGSKSVHWYQQKPGQAPVLVVYDDSDRPSWIPERFSGSNSGNTATLTISRGEAGDEADYYCQVWDSSSDHVVFGGGTKLTVL

SEQ ID NO: 15 - protein, 17 aa, synthetic construct
VIWYDGSNKHYAESVKG

SEQ ID NO: 16 - protein, 122 aa, synthetic construct
QMQLVESGGGVVQPGRSLRLSCAASGFTFRTYGMHWVRQAPGKGLEWVAVIWYDGSNKHYAESVKGRFTITRDNSKNTLNLQMNSLRAEDTAVYYCARAPQWELVHEAFDIWGQGTMVTVSS

SEQ ID NO: 17 - protein, 17 aa, synthetic construct
VIWYDGSNKHYADSVKA

SEQ ID NO: 18 - protein, 122 aa, synthetic construct
QMQLVESGGGVVQPGRSLRLSCAASGFTFRTYGMHWVRQAPGKGLEWVAVIWYDGSNKHYADSVKARFTITRDNSKNTLNLQMNSLRAEDTAVYYCARAPQWELVHEAFDIWGQGTMVTVSS

SEQ ID NO: 19 - protein, 11 aa, synthetic construct
GGQNLGSKSVH

SEQ ID NO: 20 - protein, 108 aa, synthetic construct
SYVLTQPPSVSVAPGQTARITCGGQNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 21 - protein, 11 aa, synthetic construct
GGNQLGSKSVH

SEQ ID NO: 22 - protein, 108 aa, synthetic construct
SYVLTQPPSVSVAPGQTARITCGGNQLGSKSVHWYQQKPGQAPVLVVYDDSDRPSWIPERFSGSNSGNTATLTISRGEAGDEADYYCQVWDSSSDHVVFGGGTKLTVL

SEQ ID NO: 23 - protein, 11 aa, synthetic construct
QVWDTSSDHVV

SEQ ID NO: 24 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDTSSDHVVFGGGTKLTVL

SEQ ID NO: 25 - protein, 11 aa, synthetic construct
QVWDSTSDHVV

SEQ ID NO: 26 - protein, 108 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSTSDHVVFGGGTKLTVL

SEQ ID NO: 27 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 28 - protein, 227 aa, synthetic construct
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 29 - protein, 214 aa, synthetic construct
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - DNA, 681 bp, synthetic construct
  source 1..681: mol_type = other DNA
cagatgcagttggttgaatctggtggcggcgtggtgcagcctggcagatctctgagactgtcttgtgccgcctccggcttcaccttcagaacctacggaatgcactgggtccgacaggcccctggcaaaggattggaatgggtcgccgtgatttggtacgacggctccaacaagcactacgccgactccgtgaagggcagattcaccatcaccagagacaactccaagaacaccctgaacctgcagatgaactccctgagagccgaggacaccgccgtgtactattgtgctagagcccctcagtgggaactcgtgcatgaggcctttgacatctggggccagggaacaatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgacagtgccctccagcagcttgggcacccagacctacatctgcaacgtgaatcacaagcccagcaacaccaaggtggacaagagagttgagcccaaatcttgtgacaaa

SEQ ID NO: 31 - DNA, 642 bp, synthetic construct
  source 1..642: mol_type = other DNA
tcatatgttcttacacaaccaccgtcggtttcggttgctccaggacaaacagctcgaattacatgcggaggaaacaacctcggatcgaagtcggttcactggtatcaacaaaagccaggacaagctccagttctcgtggtgtacgatgattcagatcgaccatcatggatcccagagcgattctcaggatcaaactcgggaaatactgccacgctcacaatttcacgcggagaagcgggagatgaagctgattactattgccaagtgtgggactcgtcgtcagatcatgttgttttcggaggtggaacaaagctcacagtgctcggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca